(19) United States
(12) Patent Application Publication
(10) Pub. No.: US 2010/0330044 A1
(43) Pub. Date: Dec. 30, 2010

(54) MESP1 AS A MASTER REGULATOR OF MULTIPOTENT CARDIOVASCULAR PROGENITOR SPECIFICATION AND USES THEREOF

(75) Inventors: CÉDRIC BLANPAIN, LASNE (BE); ANTOINE BONDUE, BRUXELLES (BE); GAËLLE LAPOUGE, BRUXELLES (BE)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITE LIBRE DE BRUXELLES, BRUXELLES (BD)

(21) Appl. No.: 12/494,941

(22) Filed: Jun. 30, 2009

(51) Int. Cl. A61K 45/00; C12N 5/071; G01N 33/50; C12Q 1/68; A61P 9/10

(52) U.S. Cl. 424/93.7; 435/377; 435/7.21; 435/6; 435/325

(57) ABSTRACT

A method for differentiating or promoting or inducing differentiation of stem cells into pluripotent cardiovascular progenitors (MCPs) by transiently inducing the expression of a single gene, namely Mesp1, is disclosed. Cells obtained by the method and their uses in research and clinical settings are also disclosed. Using genome wide transcriptional analysis, the inventors found that Mesp1 rapidly activates and represses a discrete set of genes, which form potential new targets for both therapy and for the identification of MCPs. Insights into the molecular mechanisms underlying the earliest step of cardiovascular specification and potential methods for dramatically increasing the number of cardiovascular cells for cellular therapy in humans are provided.

### FIELD OF THE INVENTION

The present invention relates to processes and compositions for controlling cell fate promotion from stem cells, preferably embryonic stem (ES) cells and promoting specifically and in a controlled manner cardiac and vascular differentiation from ES cells, other pluripotent or multipotent stem cell (SC) types through the specification of multipotent cardiovascular progenitor cells (MCP) as well as the use of said MCP or the differentiated cells arising from the differentiation of MCP for therapeutic and research purposes.

### BACKGROUND OF THE INVENTION

Differentiation of mouse embryonic stem cells into cardiomyocytes has historically been achieved through spontaneous differentiation of embryonic stem cells or carcinomic embryonic stem cells in serum containing medium or through treatment with compounds like DMSO, retinoic acid, bone morphogenetic proteins, fibroblast growth factors or the broad and non-specific de-methylating agent 5-aza-deoxycytidine. Compounds such as 5-aza-deoxycytidine have also been used to induce differentiation of cardiomyocytes from other stem cell types. This indicates that different types of stem cells could potentially be used as a source for the generation of cardiomyocytes for applications such as cell therapy, tissue engineering, pharmacological and toxicological screening or the like, provided effective processes and methods for the differentiation of cardiomyocytes are available. Unlike direct specification of MCP from undifferentiated cells, current methods used to promote cardiac differentiation, generally act in the latter step of cardiac expansion and differentiation, and the promotion in cardiac differentiation usually induced by a single factor rarely exceeds 100%.

When allowed to differentiate, ES cells form spherical structures called “embryoid bodies” (EBs) that are thought to mimic interactions that arise during development. This differentiation process results in the formation of beating areas around day 8, made of cardiomyocytes when differentiation is performed in serum-containing medium. Current published methods for forming cardiomyocytes from ES cells rely on spontaneous differentiation in media containing animal serum, a medium component that is largely undefined and subject to batch-to-batch variations. This method does not lend itself to being a clinically useful and reproducible system. Moreover the global efficiency of cardiac differentiation using the previously described methods is low, leading to 2-3% of cardiac cells whatever the differentiating system used.

Different lines of evidence suggest that different cardiac cell types, including cardiomyocytes (CM), endothelial cells (EC), smooth muscle cells (SMC) as well as conduction cells, which compose the mature heart tissue, arise from the differentiation of multipotent cardiovascular progenitors (MCPs) generated soon after gastrulation (Garry and Olson, 2006, Cell 127:1101-1104; Moretti et al., 2006, Cell 127, 1137-1150; Wu et al., 2006, Cell 127, 1137-1150). Recent studies also provide compelling evidence that different sources of MCPs, which are specified during embryonic development, are also generated during pluripotent embryonic stem cell (ESCs) differentiation (Moretti et al., 2006, Cell 127, 1137-1150; Murry and Keller Cell 132, 661-680, 2008; Wu et al., 2006, Cell 127, 1137-1150). Mesp1, a transcription factor of the b-HLH family, is one of the earliest markers of cardiovascular development in vertebrates (Saga et al., 2000, Trends Cardiovasc Med 10, 345-352). During gastrulation in mice, Mesp1 is strongly expressed at the onset of gastrulation (E6.5) along the primitive streak and in the prospective cardiac mesoderm and is then rapidly downregulated after E7.5 (Saga et al., 1996, Development 122, 2769-2778). Lineage tracing experiments, using mice in which the CRE recombinase has been knocked-in into the Mesp1 locus, demonstrated that most cardiac cells and some vascular cells, arise from cells that expressed Mesp1 at one point of their development (Saga et al., 1999, Development 126, 3437-3447).

The need for cardiovascular progenitors or adult cells is very high in both clinical and research settings and is currently not easily fulfilled. The current invention provides for improved means and methods to obtain such cardiovascular progenitors or differentiated cardiovascular cells, which can be safely transplanted in patients with cardiovascular diseases or used in research and industrial perspectives.

### SUMMARY OF THE INVENTION

In the present invention, Mesp1 is identified as the key molecular determinant of multipotent cardiovascular progenitors specification. It is shown by genetically engineered ESC in which Mesp1 expression can be conditionally induced that transient but not continuous expression of this gene promote greatly the specification of MCP and their different cardiovascular cell progenies including cardiomyocytes, vascular cells, cell of the conduction system, and smooth muscle cells. The inventors describe a novel method that allows a high efficient generation of cardiovascular cells from pluripotent cells by transiently expressing a single gene. Moreover they identify many MesP1 target genes that are responsible for the cardiac and vascular promoting effect of Mesp1, and which now represents key target genes for which pharmaceutical intervention will be useful to improve cardiac regeneration in acute and chronic heart failure. In addition, the technique presented here to promote cardiovascular differentiation represent an extremely versatile method for promoting cardiac cell differentiation from various sources of stem cells that can be used for cellular therapy in humans but also for, tissue engineering, pharmacological and toxicological screening

The invention therefore provides for a novel and highly efficient method for generation of cardiovascular cells or progenitors and uses thereof in research and the clinic.

In a particular embodiment, the invention provides a method of inducing, enhancing the induction or differentiation of stem cells into cardiovascular precursor or progenitor cells comprising the steps of:

a) transiently inducing the expression of the Mesp1 gene in said stem cells, and
b) culturing said induced stem cells in vitro thereby obtaining differentiated stem cells that are enriched in cardiovascular progenitor cells.
c) specifying and differentiating the cardiovascular progenitors generated by method of the invention into a particular subset of cardiovascular lineages such as cardiomyocytes, vascular or endothelial cells. Preferably, the transient expression is performed in vitro by transforming said stem cells with a vector comprising the gene sequence of the Mesp-1 protein. In another embodiment, said Mesp-1 gene sequence is placed in an inducible expression cassette such as any one of the following non-limiting examples: the Tetracyclin or doxycyclin induced systems, Rheo switch systems, FRT system, IPTG-LAC inducible systems, ecdysone inducible systems.

In another embodiment of the invention the induction of the Mesp-1 expression is performed during cardiovascular competence which need to be precise for each types of stem cells used and that correspond for murine ESC to day 2 or day 3, or day 2 and day 3 of the culturing period of the stem cells. Preferably, said induction is performed for one or two days only.

In a further embodiment, the method of the invention can use several types of cells as a starting point. Non-limiting examples are: Embryonic Stem cells (ES), pluripotent stem cells, haematopoietic stem cells, totipotent stem cells, mesenchymal stem cells, induced pluripotent stem cells (iPS) or adult stem cells, adult heart, epicardial, vessel or muscular cells.

In another embodiment, the present invention can have different potential applications. In one embodiment, the method of the invention allows for the generation of multipotent cardiovascular progenitor cells from stem cells for cellular therapy and reactivation of these progenitor in-vivo for improving the repair of cardiovascular diseases. The transient Mesp1 expression method can be used to produce high amount of cardiovascular progenitor cells that could be used for transplantion in patients or animals suffering from any condition where cardiac, vascular or conductive cells are lacking.

In a further embodiment, the invention therefore provides for a method for performing cellular therapy, comprising the steps of: a) providing cells according to the method of the invention, and b) injecting said cells into the heart or the vasculature of the subject in need thereof allowing exogenous, autologous or not, cell therapy.

In a further embodiment, the invention provides for a method for restoring the heart or vasculature function in a subject in need thereof, in an endogenous manner, comprising the step of transiently inducing the expression of the Mesp-1 protein in the cells of the heart or the vasculature. Preferably, said induction is performed by injecting the subject with an amount of an expression vector encoding for the Mesp-1 protein. In one non-limiting example, said induction is performed by injecting the subject with an amount of an expression vector encoding for the Mesp-1 protein packed in a virus or not.

In a further embodiment, the invention provides for a method for identifying target genes for therapy of cardiovascular disorders comprising the steps of: a) differentiating stem cells into cardiovascular progenitor cells according to the method of the invention, b) analysing the expression level of the genes in said cells prior to and after said induction of Mesp-1 expression in said stem cells, wherein genes that are up-regulated after the gene-induction are putative targets for stimulation of differentiation of cardiovascular differentiation and those genes that are down-regulated after the gene-induction are putative targets for inhibiting cardiovascular differentiation of stem cells. The results of such a test are given in Table 3.

In a further embodiment, the invention provides for diagnostic methods and tools for determining cardiovascular abnormalities by measuring or monitoring the expression of the Mesp1 gene or one of its target genes as defined in Table 3 below.

In a further embodiment, the invention provides for methods of treating a subject in need thereof with a therapeutically effective amount of a composition leading to increased presence of the Mesp1 protein or one or more of its targets. The composition can either be a vector encoding the Mesp1 gene under the control of an inducible promoter. When a vector system is used, this vector can be delivered to the site of therapy (e.g. the heart or vasculature where repair is needed) by methods of direct DNA or RNA injection known in the art or by infection by an attenuated viral delivery system or other DNA or RNA delivery system known in the art and usable in a clinical setting.

In another embodiment, the downstream targets of Mesp1, as identified in Table 3 below can also be of use in method of diagnosis and treatment of cardiovascular disorders. The target genes that are upregulated can be seen as alternative positive targets (activation can be of therapeutic use) for inducing cardiovascular differentiation, while the genes that are downregulated by Mesp1 can be seen as negative targets (inactivation can be of therapeutic use) for the differentiation of cardiovascular cells. The invention therefore provides methods to induce cardiovascular differentiation in stem cells by modulating the expression of one or more of the genes listed in Table 3 of the present invention. In a preferred embodiment, genes that are up- or down-regulated five fold, ten-fold or twenty-fold are preferred. In a particularly preferred embodiment, those genes are selected from the group of: Ripply2, Cited1, Trim9, Raspgrp3, Foxl2os, Tctex1d1, Hey2, Otx1, Pcsk5, DII3, Rai2, Kctd12, Caecam10, Myl1, Clstn2, Pcdh17, similar to Dhand protein, Pcdh19, Wnt5a, Ebf2, Chodl, Snap91, Hprt1, Lhfp, Pdzrn3, Brachyury, Slc35d3, Foxa2, Sox17, FgF8 and Fst.

Again, as for Mesp1 modulation, the target genes of Mesp1 can in certain emodiments of the invention be modulated by direct induction of their expression by injecting DNA or RNA (direct injection or viral transduction or other known means of delivery) encoding for the protein at the site of need, or they can be modulated by injecting a modulator (agonist or antagonist depending on whether the target is up- or down-regulated by Mesp1) of said genes or proteins at the site of need.

In a further embodiment, the invention provides for a panel of genes for detecting, quantifying, or isolating cardiovascular precursor cells based on the expression pattern of one or more of the Mesp1 modulated genes, comprising at least two genes selected from the group of genes listed in Table 3. Such a panel can according to a further aspect of the invention be used for detecting, quantifying, or isolating cardiovascular precursor cells based on the expression pattern of one or more of the Mesp1 modulated genes. Typically, the panel is in the form of a customised microarray known in the art, comprising oligonucleotides or probes that are highly specific for each of the listed genes. In a further embodiment, the panel can be in the form of a protein array known in the art.

In a further embodiment, the invention provides for the use of an Mesp-1-expressing vector under the control of an inducible promoter in the preparation of a medicament for restoring cardiovascular functioning in a subject.

In a further embodiment, the invention provides for the use of a virus particle encompassing an Mesp1 expressing vector under the control of an inducible promoter.

In a further embodiment, the invention provides for the use of cardiovascular differentiated cells obtained by the method of the invention for the preparation of a medicament for restoring cardiovascular functioning in a subject.

In a particular embodiment, said cardiovascular function is disturbed due a disease or disorder selected from the group of: Congenital Heart Disease, such as malformations and misplacements of cardiac structures, acquired heart and vascular diseases, such as myocardial infarction, cardiac hypertrophy and cardiac arrhythmia and cardiovascular damage due to trauma, although said list is exemplary and non-limiting.

In a further embodiment, the invention provides for an assay for assessing the toxicity of an agent on heart or vascular cells, comprising the steps of:

a) differentiating stem cells into cardiovascular progenitor cells according to the method of the invention, b) subjecting said cells in vitro to said agent, and c) analysing the toxic effect of said agent on the cells obtained in step a).

In a further embodiment, the invention provides for an assay for assessing the pharmacology of a candidate drug comprising the steps of: a) differentiating stem cells into cardiovascular progenitor cells according to the method of the invention, b) subjecting said cells in vitro to said candidate drug, and c) analysing the behaviour of said cells in the presence and absence of said candidate drug.

In a further embodiment, the invention provides for a method for identifying target genes for therapy of cardiovascular disorders comprising the steps of: a) differentiating stem cells into cardiovascular progenitor cells according to the method of the invention, b) analysing the expression level of the genes in said cells prior to and after said induction of Mesp-1 expression in said stem cells, wherein genes that are up-regulated after the gene-induction are putative targets for stimulation of differentiation of cardiovascular differentiation and those genes that are down-regulated after the gene-induction are putative targets for inhibiting cardiovascular differentiation of stem cells.

In another embodiment, the present invention also provides a source of cardiovascular cells for tissue engineering that can be used in the development of transplantation therapies and for research purposes.

In a further embodiment, the invention provides for the identification of genes implicated the molecular specification of multipotent cardiovascular progenitor cells from stem cells and in the repair of cardiovascular diseases. The microarray data obtained by the methods of the invention provides for the identification of novel molecular determinants for the induction of MCP from stem cells. Expression of these genes, stimulation or inhibition of these genes or their proteins, stimulates MCP specification, migration, differentiation, repair and thus will be useful for therapy aiming at repairing acute and chronic cardiac diseases as well as acute and chronic vascular diseases. The monitoring of the expression of theses genes will be also use to predict the outcome of cardiac diseases and to identify the causes of congenital and acquired cardiovascular diseases. Modulated genes/markers are summarized in the table.

In a further embodiment, the invention provides for cardiovacular cells or progenitor obtained by the method of the invention.

In a further embodiment, the invention provides for a method for diagnosis and treatment of congenital heart diseases comprising the detection of the occurrence of mutations in the Mesp1 genomic region or in the Mesp1 target genes as listed in Table 3.

In yet another embodiment, the invention further provides for method for restoring the heart or vasculature function in an endogenous manner, in a subject in need thereof, comprising the step of transiently inducing the expression of the Mesp-1 protein in the cells of the heart or the vasculature. Preferably, said induction is performed by injecting the subject with an amount of an expression vector encoding for the Mesp-1 protein.

Different congenital heart diseases are characterized by abnormal closure or malposition of cardiac structures, following specification or migration defects during embryogenesis. In a further embodiment, the invention provides new candidates (Mesp1 and its identified target genes) for diagnosis and treatment of congenital heart disease, including detection of mutations in Mesp1 coding sequence and regulatory regions, but also mutations in Mesp1 target genes. Similarly, quantification of the expression of these genes in different cardiovascular diseases will predict the cause and the clinical outcome of the disease.

In a further embodiment, the invention provides for the use of cardiovascular cells obtained by the methods as indicated above, for evaluating the cardiovascular effects of a drug on differentiated cardiac cells or for evaluating the cardiovascular effects of a drug during cardiovascular development. The invention provides for an assay for assessing the pharmacology of a candidate drug comprising the steps of: a) differentiating stem cells into cardiovascular progenitor cells according to the method of the invention, b) specifying and differentiating the cardiovascular progenitors generated by method of the invention into a particular subset of lineages such as cardiomyocytes, vascular or endothelial cells b) subjecting said cells in vitro to said candidate drug, and c) analysing the behaviour of said cells in the presence and absence of said candidate drug.

In a further embodiment, the invention provides for an assay for assessing the toxicity of an agent on heart or vascular cells, comprising the steps of: a) differentiating stem cells into cardiovascular progenitor cells according to the method of the invention, b) specifying and differentiating the cardiovascular progenitors generated by method of the invention into a particular subset of lineages such as cardiomyocytes, vascular or endothelial cells c) subjecting said cells in vitro to said agent, and d) analysing the toxic effect of said agent on the cells obtained in step a and b).

In a further embodiment, the invention provides for cardiovascular progenitor cells or cardiovascular cells obtained by the methods of the invention.

### BRIEF DESCRIPTION OF THE DRAWINGS

FIG. 1. Precocious expression of Mesp1 dramatically accelerates and increases cardiac differentiation from ES cells. (A) Expression profiles of early mesodermal (Brachyury and Mesp1) and cardiac transcripts (Nkx2.5, Gata4, Mef2c, Hand2 and TroponinT2) during normal ES cell differentiation as measured by RT-PCR. Upon differentiation, genes regulating the specification of the primitive streak such as Brachyury are rapidly upregulated. Mesp1 is expressed soon after and peaks transiently at D4, before the appearance of cardiac genes. (B) Acceleration of cardiac differentiation upon transient Mesp1 expression. A pulse of Mesp1 (D2-D3), before its endogenous expression, accelerates cardiac commitment in EBs as detected by the appearance of beating areas as early as day 6, compared to day 8 in unstimulated or GFP overexpressing cells, while a continuous administration of Dox inhibits cardiac differentiation. (C) Immunostainings for troponinT: following differentiation, EBs were fixed and stained using a troponinT antibody, a specific marker of cardiomyocytes. Mesp1 expression during D2-D3 induced a dramatic acceleration in troponinT expression. Squares indicate areas of magnification for each condition shown on the right of the panel. (D and E) Quantification of troponinT expression during ESC differentiation by FACS analysis. At different times of differentiation, EBs were dissociated, stained for troponinT and analysed by FACS. These data show the dramatic acceleration and increase in cardiac differentiation following Mesp1 expression. (F) Absolute enrichment in troponinT positive cells following a Mesp1 transient expression. The percentage of troponinT positive cells was adjusted for the total number of cells presented in both conditions (see FIG. 10).

FIG. 2. Mesp1 specifically promotes multipotent cardiovascular progenitor cell fate. (A) Expression of cardiovascular markers analyzed by RT-PCR after 10 days of differentiation in Mesp1 stimulated cells (black bars) versus control (white bars). Gene expression profiles after 10 days of differentiation of Mesp1 expressing cells compared to unstimulated cells. These data demonstrates the enhancement in cardiac transcription factors (Nkx2.5, Gata4, Tbx5 and Tbx20), pancardiac (TroponinT2, β-MHC), atrial (Mlc2a and ANF), ventricular (Mlc2v) and conductive cells markers (Kcne1) in Mesp1 overexpressing EBs. Mesp1 also increases the expression of CD31, a vascular marker. (B to E) Immunostainings of EBs using ventricular (mlc2v-B), atrial (Mlc2a-C) and vascular (VE-cadherin-D) or smooth muscle cell markers (smooth muscle actin-SMA-E). These immunostainings demonstrate an acceleration and an enhancement in atrial, ventricular and vascular markers expression in Mesp1 stimulated EBs. Squares indicate areas of magnification for each condition represented on the right of the panel. (F) Expression of Islet1 as measured by RT-PCR following Mesp1 induction. Mesp1 induces a transient enhancement of Islet1 expression in EBs. Results are normalized for the expression of unstimulated cells. (G) Immunostainings of Islet1 in EBs demonstrates a precocious expression of islet1 at day 6 in Mesp1 induced EBs similar to its expression at day 8 in unstimulated EBs. (H) Expression of other mesodermal, endodermal and neurectodermal markers analyzed by RT-PCR after 10 days of differentiation in Mesp1 stimulated cells (black bars) versus control (white bars). These data show an increase in expression of hepatic markers (Albumin, a-foetoprotein, and TCF1) and striated muscle markers (Myogenin) while other endodermal markers (Pdx1, Sox17, Sox18 and Gata6), bone markers (runx2, Col1A1), epitelial markers (K8, K14 and K18) or neuronal markers (Tuj1, nestin, and Sox1) were unchanged or relatively decreased.

FIG. 3. Mesp1 specifically promotes cardiac progenitor cell fate by an intrinsic and cell autonomous mechanism. (A) Schematic representation of the experimental procedure using conditioned media (CM). We collected CM of Mesp1 stimulated cells daily, transferred it to control cells and analyzed cardiac differentiation over time. (B) Percentage of beating EBs over time following addition of Mesp1 CM. Addition of Mesp1 CM did not promote cardiac differentiation to naïve cells. Controls are Mesp1 stimulated cells and naïve cells receiving CM from Mesp1 unstimulated cells. (C) Schematic representation of the experimental procedure using EBs cocultures: Mesp1-IRES-GFP expressing EBs are co-cultured with control EBs expressing DsRed alone. EBs are plated in the same well 24 hours after Dox addition. (D) Percentage of beating EBs in mixed EBs experiment. (E) Quantification of troponinT expression in the mixed EBs experiment at day 8 of differentiation as measured by FACS analysis. At day 8, EBs are dissociated and stained using troponinT antibody, and the percentage of troponinT positive cells was determined in Mesp1 (GFP positive) or in control (DsRed positive) cells. These experiments show that cardiac promotion is observed only in Mesp1 expressing cells (GFP). (F) Schematic representation of chimeric EB experiment. Equivalent cell number of Mesp1-IRES-GFP cells or control GFP ES cells were mixed with DsRed expressing cells.(G) TroponinT expression in Mesp1 expressing cells (GFP positive), mixed with DsRed cells as measured by FACS analysis after 8 days of differentiation. Percentage of troponin T positive cells is much higher in Mesp1 stimulated cells than in RFP stimulated cells or in RFP cells that have been in contact with GFP cells, demonstrating that Mesp1 promotes cardiac specification mainly through a cellular autonomous mechanism.

FIG. 4. Mesp1 promotes multipotent progenitor cardiac cell fate by directly promoting the expression of the core cardiac transcriptional machinery. (A) Temporal expression of cardiovascular transcription factors Hand2, Myocardin, Nkx2.5, Gata4, Mef2c, FoxH1, FoxC1 and FoxC2 following a transient Mesp1 induction. These results demonstrate a rapid modulation of these genes already detectable as early as 12-18 hours post Dox stimulation. (B-D-F-H) Representation of genomic region surrounding Hand2 (B), Myocardin (D), Nkx2.5 (F) and Gata4 (H) genes. Untranslated regions are depicted in yellow. Exons are shown by wide blue lines and intron by thin blue lines. The previously described cardiac enhancers are highlighted in green (Lien et al., 1999; McFadden et al., 2000; Searcy et al., 1998). Conserved EBox sites between human and mouse sequences and relative position of PCR fragments used to measure the enrichment following ChIP are shown. C-E-G-I-Quantification of DNA fragments enrichment by ChIP using anti-Mesp1 antibody relative to control isotype antibody as measured by RT-PCR for Hand2 (C), Myocardin (E), Nkx2.5 (G) and Gata4 (I).

FIG. 5. Mesp1 represses the expression of genes regulating pluripotency, early mesoderm and endoderm cell fates. A and B-. Expression of Eras and Id2 (A), Oct4, Nanog and Sox2 (B) mRNAs, following a transient Mesp1 expression determined by RT-PCR. Results are normalized for expression in unstimulated cells at the same day of differentiation. Note the more rapid downregulation of Eras and Id2 expression (A) compared to Oct4, nanog and Sox2 expression (B). C-Immunostainings for Nanog on cytospins after dissociation of embryoid bodies 48 hours after Dox stimulation (day 4) D-Temporal expression of Sox17, Foxa2, Brachyury, FGF8, Gsc, Cer1 and Nodal following Mesp1 induction using RT-PCR analysis showing the rapid downregulation of these genes following Mesp1 induction. E-Immunostaining for Foxa2 on replated EBs at day 6 showed the downregulation in Foxa2 expression following Mesp1 induction. F-H-J-L-Representation of genomic region surrounding Foxa2 (F), Gsc (H), Sox17 (J) and Brachyury (L) genes. Untranslated regions are depicted in yellow. Exons are shown by wide blue lines and introns by thin blue lines. Conserved Ebox sites between human and mouse sequences and relative position of PCR fragments used to measure the enrichment following ChIP are shown in orange. G-I-K-M Quantification of DNA fragments enrichment by ChIP using anti-Mesp antibody relative to control isotype antibody as measured by RT-PCR for Foxa2 (G), Gsc (I), Sox17 (K) and Brachyury (M).

FIG. 6. Mesp1 regulates its own expression through a complex gene regulatory circuit. (A) Temporal expression of Ripply2 following Mesp1 expression as determined by RT-PCR analysis. Ripply2 expression is strongly and rapidly upregulated following Mesp1 induction. (B) Representation of the genomic region surrounding the Ripply2 gene. Untranslated regions are depicted in yellow. Exons are shown by wide blue lines and introns by thin blue lines. Conserved EBox sites between human and mouse sequences and relative position of PCR fragments used to measure the enrichment following ChIP are shown. (C) Quantification of DNA fragments in the Ripply2 gene enriched by ChIP using anti-Mesp antibody relative to control isotype as measured by RT-PCR. Mesp1 IP enriches by 20 fold a DNA fragment 6.5 kB upstream of the start translation site. (D) Temporal expression of endogenous Mesp1 and Mesp2 following Mesp1 expression by RT-PCR analysis. Endogenous Mesp1 transcript is specifically detected by PCR of the 3′ UTR region of Mesp1, which is not presented in the inducible construct. Note the biphasic effect of Mesp1 on its endogenous expression. (E) Representation of the genomic region surrounding the Mesp1 gene. Untranslated regions are depicted in yellow. Exons are shown by wide blue lines and introns by thin blue lines. Conserved EBox sites between human and mouse sequences and relative position of primer pairs used to measure the enrichment following ChIP are shown. (F) Quantification of DNA fragments enrichment by ChIP using anti-Mesp antibody relative to control isotype measured by RT-PCR for Mesp1.

FIG. 7. Model of Mesp1 functions during multipotent cardiovascular progenitor specification. We proposed a model in which Mesp1 acts as a molecular switch to promote cardiovascular specification from undifferentiated mesoderm, by directly stimulating the expression of most key cardiac transcription factors of the primary and secondary heart field. Mesp1 directly repressed the key transcription factors controlling alternate cell fate during this stage of differentiation. Mesp1 first stimulates its own expression through a positive auto regulatory loop followed by a subsequent repression of Mesp1, ensuring the strong and transient Mesp1 expression, and thus acting as a molecular switch during cardiovascular specification.

FIG. 8. Recombinant ES cells allowing Dox inducible expression of a C-terminus flagged Mesp1 IRES GFP transgene. (A) Schematic representation of the experimental procedures used for the generation of an ES cell line allowing a Dox inducible expression of Mesp1. Mesp1-ORF is cloned in frame with a 3XFlag, followed by a double stop codon followed by an IRES-EGFP in the p2Lox vector backbone. As previously described, this entry vector is co-electroporated with the pSalCre vector in a modified ES cell line (MI and MK, manuscript submitted) allowing a Dox inducible transgene expression after clone selection (Kyba et al., 2002). (B) Kinetic of GFP expression as measured by FACS after dox induction in EBs. (C) Kinetic of flagged-Mesp1 expression by immunostaining using anti-Flag M2 antibody. EBs are dissociated at different times following Dox addition. Mesp1 is undetectable in the absence of Dox and becomes detectable as soon as 6 hours after stimulation, localizes in the nucleus after 12 hours and is expressed in about 80% of cells 24 hours post stimulation. (D) Kinetic of flagged-Mesp1 expression using western blotting with anti-Flag M2 antibody. While undetectable in basal conditions, Mesp1 is rapidly upregulated after Dox stimulation. (E) Expression levels of endogenous Mesp1 peak as measured by RT-PCR at day 4 of differentiation and transgene expression after 24 hours of stimulation using Dox at 100 ng/ml and 1 μg/ml. Results are normalized for expression in ESCs. Stimulation of cells with 100 ng/ml of Dox results in a lower Mesp1 expression than the physiological level, while Mesp1 expression using 1 μg/ml of Dox is similar to the peak of endogenous Mesp1.

FIG. 9. (A) MF20 Immunostaining following Mesp1 induction. EBs were fixed and stained using an anti-β-MHC antibody (MF20). Mesp1 expression induces a dramatic acceleration and enhancement of muscle differentiation. (B) Triple immunostaining against Nkx2-5, Flk1 and Islet1 demonstrating MCP specification in Mesp1 induced EBs at D6 of differentiation. Arrows indicate triple positive cells.

FIG. 10. Effect of Mesp1 on cardiac progenitor cells expansion. (A) Pictures of embryonic bodies 24 hours and 48 hours following Dox stimulation. Mesp1 overexpressing EBs are bigger in size. (B) Cell counts of dissociated embryonic bodies following transient Mesp1 expression. Compared to controls, Mesp1 expressing cells show only a transient growth advantage during the first 48 hours post stimulation. (C) Cell cycle analysis of control and stimulated Mesp1 expressing cells measured by BrdU incorporation and DNA content using FACS, 48 hours after Dox stimulation. (D) Active caspase-3 activity in cells measured by FACS 48 hours after Dox stimulation. Note the reduction of apoptosis in Mesp1 expressing cells.

FIG. 11. (A to E) PCR amplification of genomic DNA fragments after ChIP using anti-Mesp antibody compared to control isotype for Hand2 (A), Myocardin (B), Nkx2.5 (C), Gata4 (D) and Mef2c (E) after 40 cycles. (F) Mesp1 induces reporter activity of Hand2 enhancer regions (regions L+M and D+E) inserted into a luciferase reporter construct containing a minimal promoter in 293 cells. No stimulation is observed in reporters that do not contain any enhancer or conserved EBox sites (region G). Results are normalized to luciferase expression in cells not transfected with Mesp1.

FIG. 12. (A to D) PCR amplification of genomic DNA fragments after ChIP using anti-Mesp antibody compared to control isotype for Foxa2 (A), GSC (B), Sox17 (C) and Brachyury (D) after 40 cycles.

FIG. 13. (A) Representation of genomic region surrounding Dkk1 gene. Untranslated regions are depicted in light grey. Exons are shown by wide lines and introns by thin lines. Conserved Ebox sites between human and mouse sequences and relative position of primer pairs used to measure the enrichment following ChIP are shown. (B) Quantification of DNA fragments enrichment by ChIP using anti-Mesp antibody relative to control isotype measured by RT-PCR for Mesp1. (C) Induction of Mesp1 expression following 24h of Wnt3a addition in wt cells as measured by RT-PCR at day 3. Results are normalized for Mesp1 expression in untreated cells at day 3.

FIG. 14. (A and B) PCR amplification of genomic DNA fragments after ChIP using anti-Mesp antibody compared to control isotype for Ripply2 (A), and Mesp1 (B) after 40 cycles.

### DETAILED DESCRIPTION OF THE PREFERRED EMBODIMENTS

In order to provide improved means and methods to differentiate stem cells into cardiovascular progenitor cells, the inventors investigated the signaling pathways involved in this differentiation process. More particularly, the inventors investigated whether Mesp1 instructs the primitive mesoderm to become multipotent cardiovascular progenitors (MCPs) and demonstrated that Mesp1 acts in a cell autonomous manner to promote multipotent cardiovascular progenitor specification, and further cardiac and vascular differentiation from pluripotent cells.

The inventors showed that, during ESC differentiation, Mesp1 is expressed very transiently, reminiscent of its expression during embryonic development. The inventors discovered that by transiently forcing the expression of Mesp1 during ESC differentiation, i.e. using an inducible expression system, the cardiovascular cell fate is drastically accelerated and promoted specifically, through a cellular autonomous mechanism. This is however not the case when constitutively Mesp1 overexpression is used. The method used by the inventors does not require addition of factors other than Mesp1 to promote MCP specification and differentiation in contrast to the method used in Lindsley et al (Cell Stem Cell, 2008, Vol. 3(1):55-68), that required Dkk1 addition to obtain cardiovascular differentiation of ES cells. In addition, using genome wide transcriptional analysis after Mesp1 induction, a discrete set of genes was uncovered, which are rapidly regulated upon Mesp1 induction (Table 3). The inventors further established that Mesp1 both directly activates many key genes belonging to the core cardiac transcriptional machinery and directly represses genes promoting pluripotency, early mesoderm and endoderm cell-fate specification. Moreover, Mesp1 first transiently stimulates its own endogenous expression through a direct positive auto-upregulatory loop, and then inhibits its own expression, therefore acting as a molecular switch during cardiac specification. Altogether, these results provide compelling evidence that Mesp1 acts as a cardiovascular master gene during specification of MCPs during ESC differentiation. This study is the first work showing a dramatic cardiovascular enhancement (by 4 to 5 fold) and acceleration (by 24-36 hours) following the addition of a single factor. These findings open a new range of possible applications related to generation and isolation of cardiovascular progenitor cells and their use.

Using an ES cell line, in which Mesp1 (NM_{—}008588.1) expression can be temporally regulated, the present invention demonstrates that only transient, but not continuous Mesp1 expression leads to a major enhancement and acceleration of cardiac differentiation during ES cell differentiation.

This effect is mediated by a direct activation of cardiovascular genes and a direct repression of genes associated with other cell fates and pluripotency (see Table 3). This repression of pluripotency induced by Mesp1 precludes the use of a constitutive expression system, such as the one described by David et al (Nature Cell Biology 2008; 10 (3): 338-45) to generate a large amount of ES cells that can be used to produce in large scale cardiac cells.

The transient Mesp1 expression represents a novel and highly efficient method that leads to massive cardiovascular differentiation by the promotion of multipotent cardiovascular progenitors from pluripotent cells.

The present invention provides a novel and highly efficient method for generation of multipotent cardiovascular progenitor cells and their differentiation into mature functional cardiac, vascular and endothelial cells. The results that describe how Mesp1 promotes cardiovascular cell fate are now elaborated in more detail below.

### Mesp1 Acts Intrinsically to Promote Multipotent Cardiovascular Progenitor Specification From Undifferentiated Mesoderm.

The inventors could demonstrate that Mesp1 acts in a cellular autonomous manner to promote multipotent cardiovascular progenitor cell specification from early mesodermal cells. The inventors revealed that precocious and transient expression of Mesp1 two days earlier (D2) than its endogenous expression (D4), before the presence of cardiovascular progenitors, accelerated cardiac differentiation by precisely two days, strongly suggesting that Mesp1 directly promotes cardiovascular progenitor cell fate specification rather than promoting progenitor expansion or differentiation. The results show that Mesp1 promotes the specification of MCPs from the primary and secondary heart fields as demonstrated by the massive increase in cardiac, endothelial and smooth muscle cells and upregulation of markers common of both sources of cells (eg: Nkx2-5, Gata4, Hand2, Tbx20, FoxH1) and specific for the primary (eg: Tbx5) and the secondary (eg: Isl1) heart field following Mesp1 induction.

Very recently, it has been proposed that Mesp1 induces cardiac differentiation through the secretion of Dkk1, a soluble Wnt inhibitor, suggesting that cardiac fate specification induced by Mesp1 occurred through a cellular non-autonomous mechanism (David et al., 2008). However, in this study the different functional experiments using conditioned medium from Mesp1 stimulated cells, co-culture of Mesp1 and control EBs, as well as the co-differentiation of chimeric EBs consisting of Mesp1 and control ESCs, demonstrate unambiguously that cardiovascular specification induced by Mesp1 is mediated predominantly through an intrinsic and cellular autonomous mechanism. Data are consistent with the in-vivo chimeric studies showing that Mesp1/2 null cells cannot contribute to the cardiac lineage despite the presence of wild type cells, demonstrating the cellular autonomous role of Mesp1/2 during mouse cardiac development (Kitajima et al., 2000). These novel data, as well as the in-vivo data, demonstrate that Mesp1 acts in a cellular autonomous manner during cardiovascular specification.

### Mesp1 Directly Promotes the Expression of Key Components of the Core Cardiac Transcriptional Machinery.

The core cardiac transcriptional machinery is composed of an evolutionarily conserved set of transcription factors belonging to the Nkx (eg: Nkx2-5), Gata (eg: Gata4), Mef (eg: Mef2c), Tbx (eg: TbxS), Hand (eg: Hand2), Myocardin/SRF (eg: Myocardin) transcription factor families, which reinforce each other's expression and stimulate alone or in combination the expression of genes required for proper cardiac development (Davidson and Erwin, 2006; Olson, 2006). Little is known about the upstream factors that initiate expression of these key cardiac transcription factors during development, resulting in their co-regulated expression in the cardiac crescent of the primary and secondary heart fields.

The inventors could now unambiguously demonstrate that Mesp1 rapidly and strongly stimulates the expression of Hand2, Myocardin, Nkx2-5, Gata4, Mef2c, Tbx20, or FoxH1. Chromatin-immuno-precipitation (ChIP) experiments showed that Mesp1 binds directly the previously described cardiac enhancers of Hand2 and Nkx2-5 (Lien et al., 1999; McFadden et al., 2000; Searcy et al., 1998), and in different regulatory regions of the proximal promoter of Myocardin and Gata4. The strength of Mesp1 binding to the regulatory regions of these genes correlates well with the importance and the rapidity of their upregulation following Mesp1 expression, strongly suggesting that Mesp1 directly regulated their transcription. The inventors now could place Mesp1 at the top of the transcriptional network that regulates cardiac differentiation, by directly coordinating the expression of the vast majority of key cardiac transcription factors at the right place and at the right time.

The transient expression of Mesp1 is sufficient to initiate the expression of these cardiac transcription factors, and as they positively regulate each other's expression, the gene network they form results in the sustained expression of these cardiac transcription factors despite the transient expression of Mesp1. The novel method does not require addition of any extrinsic factor such as Dkk1 following transient Mesp1 expression to initiate cardiac gene expression and MCP specification, in contrast to the method described in Lindsley et al. (Cell Stem Cell, 2008, Vol. 3(1):55-68).

### Mesp1 Directly Represses the Transcription of Genes Regulating Pluripotency and Alternate Cell Fates

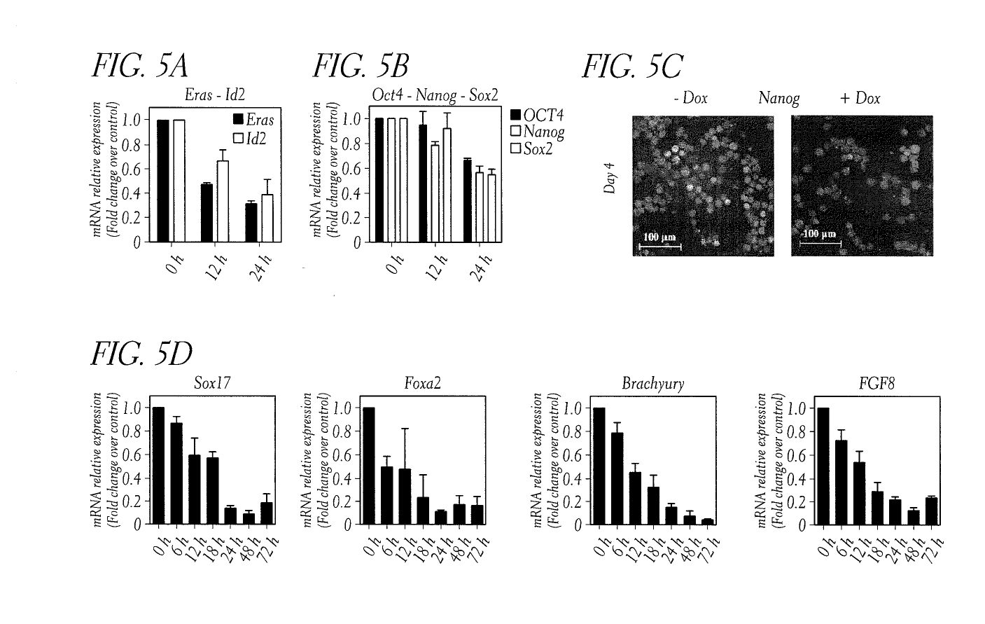

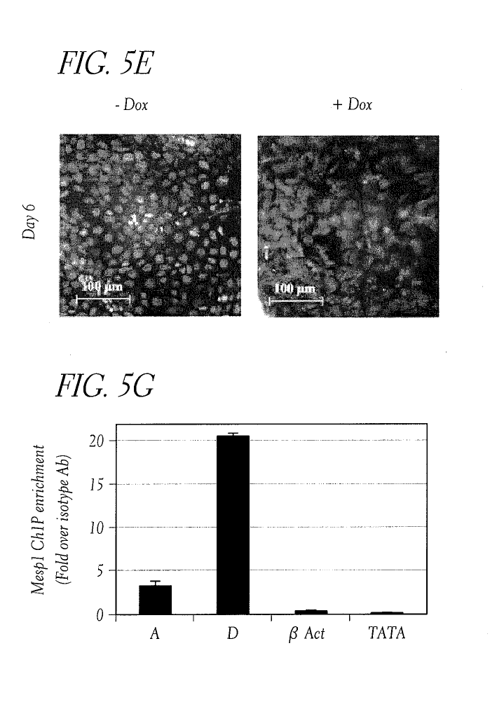

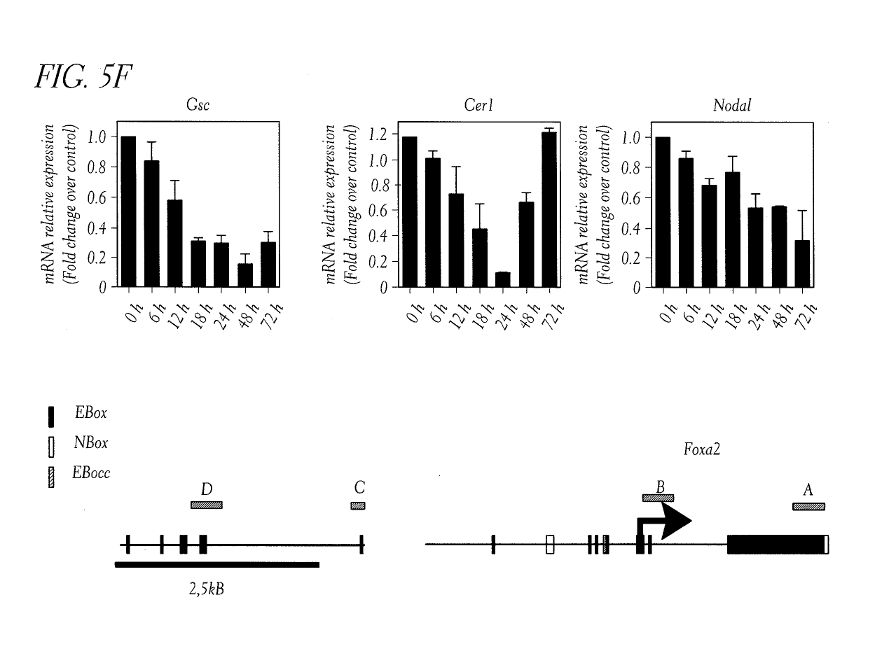

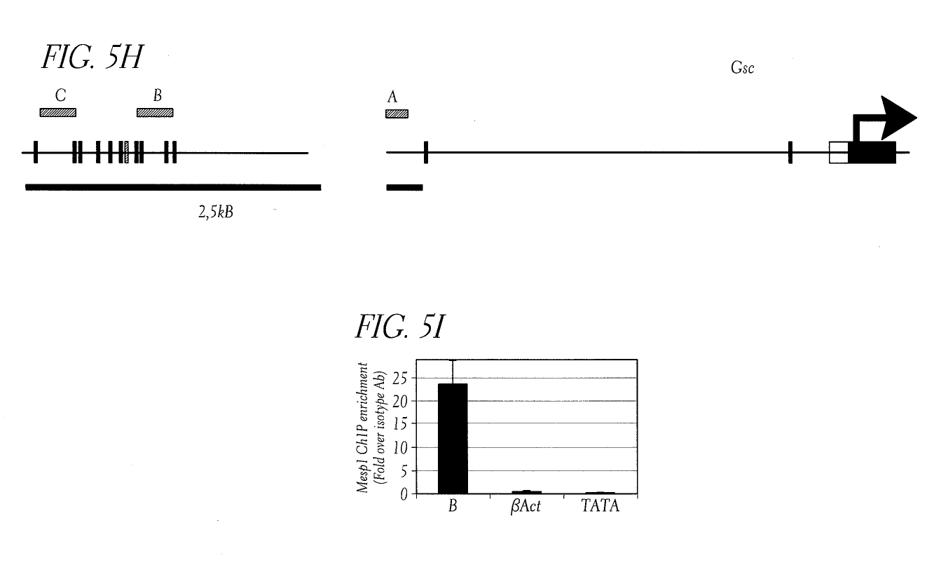

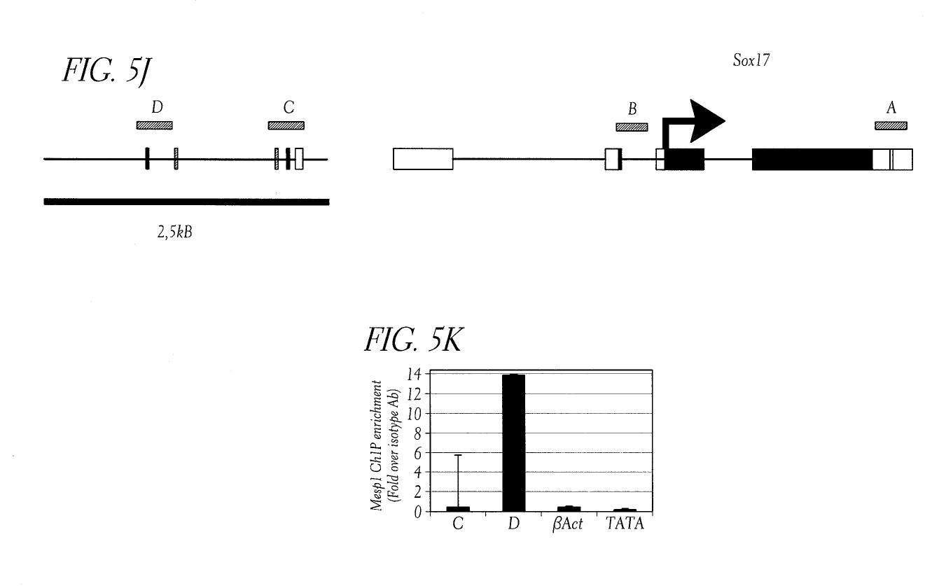

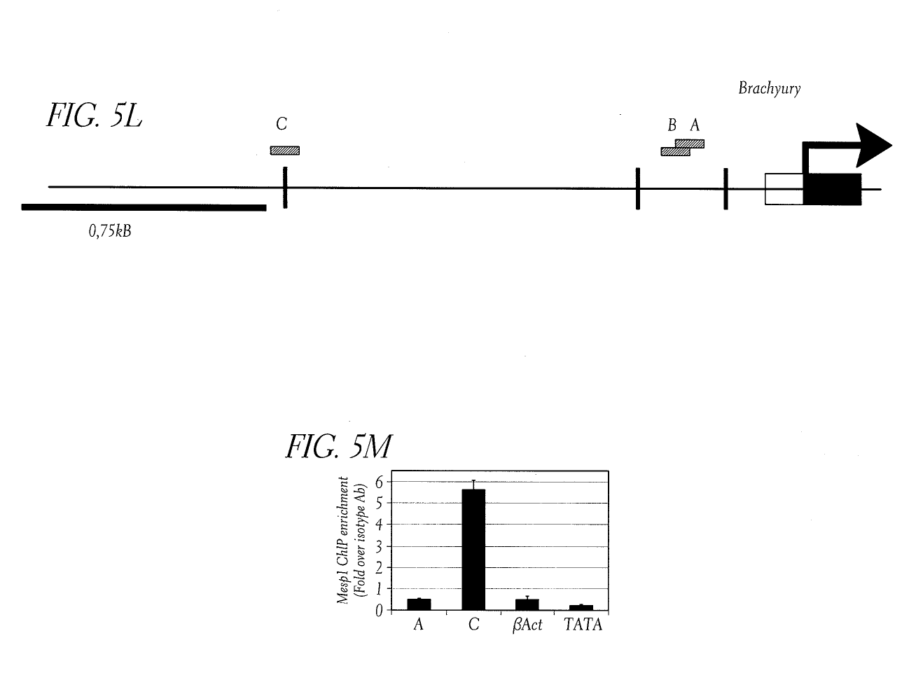

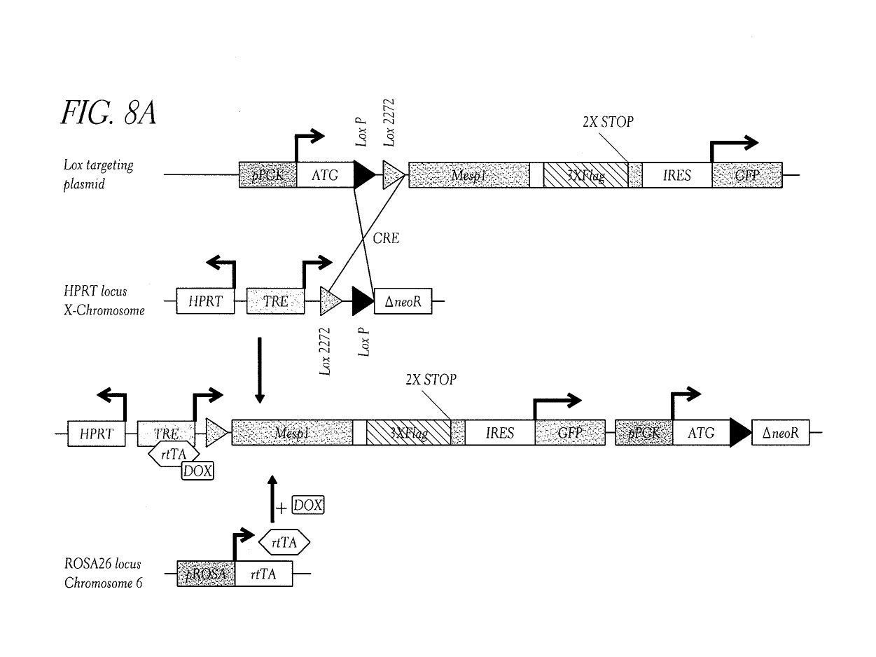

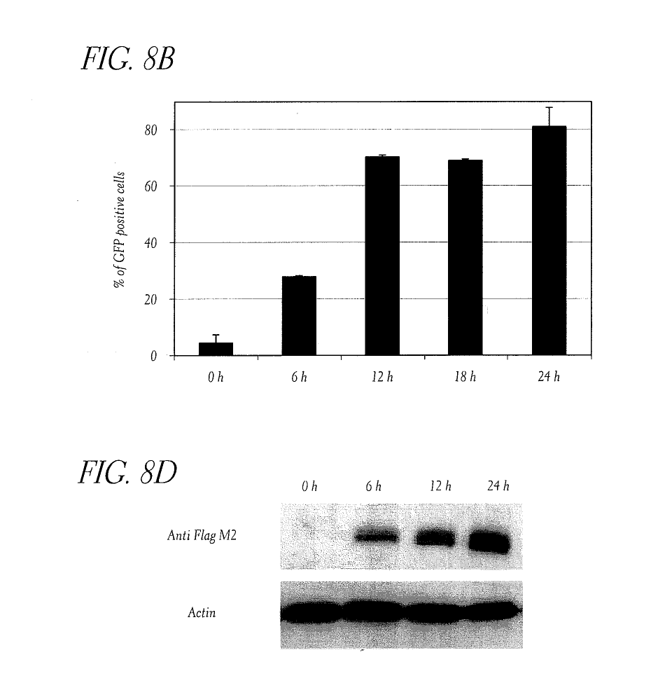

Mesp1 down-regulated the expression of about a hundred genes. The genes down-regulated by Mesp1 included many genes involved in the maintenance of pluripotency and the specification of early mesoderm and endoderm cell fate (FIGS. 5 and 8). Mesp1 downregulates the expression of Eras and Id2, but also Oct4, Nanog and Sox2, all genes that are involved in the maintenance of pluripotency of stem cells. This repression of pluripotency induced by Mesp1 precludes the use of a constitutive expression system, such as the one described by David et al (Nature Cell Biology 2008; 10 (3): 338-45) to generate a large amount of ES cells that can be used to produce in large scale cardiac cells. Moreover Mesp1 downregulates directly the expression of Brachyury and FGF8, which both act during early primitive streak specification (Huber et al., 2004; Tam et al., 2003; Tam and Loebel, 2007), as well as Foxa2, Sox17, Gsc, Nodal, and Cer1, which all function during endoderm specification (Tam et al., 2003; Tam and Loebel, 2007). The temporal analysis of these genes expression following Mesp1 induction, demonstrates that some genes (eg: Brachyury, Foxa2, Gsc and Sox17) were already strongly repressed only a few hours after the presence of Mesp1 in the nucleus (FIG. 5). These data demonstrate that Mesp1 actually directly represses these genes. The repression of these early mesodermal and endodermal genes by Mesp1 may ensure that Mesp1 induces specifically, unidirectionaly and irreversibly, the promotion of cardiovascular specification and inhibits the acquisition of other possible cell fates during this developmental stage, leading to a highly efficient method to generate cardiovascular cells from pluripotent cells.

### Mesp1 Negatively Regulates its Expression Through a Complex Gene Regulatory Network.

The inventors further demonstrated that Mesp1 very rapidly but transiently stimulated its own endogenous expression, probably through a direct mechanism as suggested by the ChIP experiments. This transient increase in Mesp1 expression is followed by a sustained and profound downregulation of its own endogenous expression, as well as the expression of its closest homologue Mesp2. The strongest upregulated gene following Mesp1 stimulation is Ripply2, a transcriptional co-repressor containing a WRPW motif (Kawamura et al., 2008).

These positive and then negative autoregulatory loops of Mesp1 expression ensure that Mesp1 acts as a gene regulatory switch during cardiovascular specification during embryonic development and ESCs differentiation.

These results demonstrate that only transient expression (and not constitutive expression as suggested by others e.g. David et al., 2008) of Mesp1 during cardiovascular competence of ESC differentiation promotes and is sufficient (in contrast to suggested by others e.g. Lindsley et al., 2008) to induce cardiovascular specification in a cell autonomous manner by promoting the cardiovascular core transcriptional machinery and by repressing alternative cell fates, and should be considered as method of choice to achieve robust and reproducible cardiovascular enrichment from ESCs.

### Mesp1 Regulates the Expression of Many Other Regulators of Cardiovascular Progenitors Functions.

Mesp1 exert its different functions through the regulation of its target genes expression. Our microarray revealed that Mesp1 also directly and rapidly regulated the expression of many key genes required for cardiovascular progenitor migration, proliferation, patterning and differentiation. Expression or inhibition of these genes can recapitulate or inhibit specific function of Mesp1 on cardiovascular progenitor specification, expansion, migration and differentiation into mature cardiac and vascular cells. Activation and inhibition of these novel regulator of cardiovascular progenitors will be useful in the treatment of a variety of cardiovascular diseases in which cardiovascular repair or remodeling are affected. See Table 3 for the list of genes.

The invention provides novel and important insights into the molecular mechanisms that promote the specification of MCPs from undifferentiated mesoderm and demonstrate that Mesp1 acts as a key molecular switch during this process, residing at the top of the hierarchy of the cardiovascular transcriptional network and stimulating the coordinated expression of the main transcription factors necessary for cardiovascular development. The genome wide transcriptional analysis of Mesp1 target genes performed in the present invention provides a comprehensive analysis of the earliest molecular mechanisms controlling cardiovascular commitment, which will constitute a framework for further exploration of the complex transcriptional network involved in cardiovascular progenitor specification.

The high efficiency method of the invention to generate cardiac cells from a potentially high number of cells in culture opens new industrial and therapeutical perspectives in e.g. cardiovascular regeneration, cellular transplantation, toxicology and pharmacology studies, isolation of cardiovascular progenitors for both clinical and research purposes, treatment and studying congenital heart disease (CHD), characterization before and after differentiation, animal models of human disease, etc.

In a first aspect of the present invention there is provided a method of inducing or enhancing the differentiation of stem cells into cardiovascular precursor cells comprising the steps of: a) transiently inducing the expression of the Mesp1 gene in said stem cells, and b) culturing said induced stem cells in vitro thereby obtaining differentiated stem cells that are enriched in cardiovascular progenitor cells.

In a preferred embodiment, the transient expression is performed by transforming said stem cells with a vector or modified virus comprising the gene sequence of the Mesp-1 protein. In a further embodiment, said Mesp-1 gene sequence is placed in an inducible expression cassette, such as commercially accessible expression cassettes chosen from the group of the Tetracyclin or doxycyclin induced systems, Rheo switch systems, IPTG-LAC inducible systems, ecdysone inducible systems, dimerization/reconstitution system or the cumate repressor/operator systems.

In another embodiment, the induction of the Mesp-1 expression is performed at day 2 or day 3, or day 2 and day 3 of the culturing period of the stem cells and preferably the induction is performed for one or two days only.

### Potential Cells to be Used in the Method of the Present Invention.

In yet a further embodiment of the invention, the stem cells are selected from the group of: Embryonic Stem cells (ES), pluripotent stem cells, haematopoietic stem cells, totipotent stem cells, mesenchymal stem cells, induced pluripotent stem cells (iPS) or other adult stem cells. In another embodiment, the cells are adult heart, epicardial, vessel or muscular cells.

### Potential Applications of the Methods of the Invention

In a first aspect, the method of the invention can be used for the generation of cardiovascular cells for cellular therapy and cell transplantation. Cardiovascular disease remains the first cause of death in western countries. Cardiovascular differentiation from pluripotent cells offers great promises for cellular therapy in humans as well as a source of cardiac cells for drug screening. In addition, the discovery that transient expression of only four transcription factors (Oct4, Sox2, myc and Klf4) can induce the reprogramming of skin fibroblasts into pluripotent embryonic like cells (Yamanaka, 2007), opens new avenues to generate induced autologous pluripotent cells from patients suffering from various cardiac diseases, and to generate cardiovascular cells for cell-therapy aiming to repair the damaged heart.

The present invention has important implications for these clinical applications, in which increasing the efficiency of cardiovascular differentiation would be needed to be useful in practice. By temporally regulating the expression of Mesp1 at different times along cardiac differentiation, the inventors demonstrated that Mesp1 specifies cardiovascular cell fate only during a restricted period of time, suggesting that ESCs are only competent to give rise to cardiovascular lineages during the early mesodermal stage of differentiation. In addition, the inventors demonstrated that only a transient pulse of Mesp1 resulted in acceleration and increase in cardiac differentiation while continuous Mesp1 expression resulted in the inhibition of cardiomyogenesis. Moreover, the inventors showed that Mesp1 expression resulted in the downregulation of many genes implicated in the maintenance of pluripotency. Thus constitutive expression of Mesp1 is likely to result in the inability to maintain ESC selfrenewal and pluripotency and precludes pluripotent cell growth before induced cardiovascular differentiation and represents a major obstacle for large scale cardiovascular cells production. Moreover transient expression of MesP1 is sufficient to induce cardiovascular gene expression and does not require addition of other factors in the culture system.

The transient Mesp1 expression method can be used to produce high amount of cardiovascular cells that could be transplanted in patients or animals suffering from any condition where cardiac, vascular or conductive cells are lacking. The invention further provides for a method for performing cellular therapy, comprising the steps of: a) providing cells according to the method of the invention, b) specifying and differentiating the cardiovascular progenitors generated by method of the invention into a particular subset of cardiovascular lineages such as cardiomyocytes, vascular or endothelial cells and c) injecting said cells into the heart or the vasculature of the subject in need thereof allowing exogenous, autologous or not, cell therapy.

The invention also provides for a method for identifying target genes for therapy of cardiovascular disorders comprising the steps of: a) differentiating stem cells into cardiovascular progenitor cells according to the method of the invention, b) specifying and differentiating the cardiovascular progenitors generated by method of the invention into a particular subset of cardiovascular lineages such as cardiomyocytes, vascular or endothelial cells b) analysing the expression level of the genes in said cells prior to and after said induction of Mesp-1 expression in said stem cells, wherein genes that are up-regulated after the gene-induction are putative targets for stimulation of differentiation of cardiovascular differentiation and those genes that are down-regulated after the gene-induction are putative targets for inhibiting cardiovascular differentiation of stem cells.

Adult cardiac progenitors have been recently isolated in adult heart. Recent evidence suggested that these cells can be activated and recruited to following cardiac injury and contribute to cardiac repair. The methods of the invention therefore make it possible to induce differentiation of said adult progenitor cells in the adult heart through the promotion of Mesp1 expression and activation of multipotent endogenous cardiovascular progenitor increasing their regenerative potential. Our discovery opens new perspectives in the recruitment, amplification, migration and differentiation processes of these multipotent endogenous progenitors following cardiac injury. In such embodiment, the invention further provides for method for restoring the heart or vasculature function in an endogenous manner, in a subject in need thereof, comprising the step of transiently inducing the expression of the Mesp-1 protein in the cells of the heart or the vasculature. Preferably, said induction is performed by injecting the subject with an amount of an expression vector encoding for the Mesp-1 protein or its target genes. Alternatively, said induction is performed by injecting a factor or an agent inducing the expression of Mesp-1 target genes in said cells of the heart or the vasculature.

To uncover the molecular mechanisms by which Mesp1 induced cardiovascular specification, the inventors performed a genome wide analysis of Mesp1 regulated genes. The inventors determined which genes were regulated upon Mesp1 induction, by at least 1.5 fold (see Table 3). A functional annotation clustering of Mesp1 regulated genes revealed that Mesp1 preferentially regulated genes implicated in morphogenesis and development (enrichment score=16.1 fold), tube morphogenesis and vessel development (7 fold), membrane proteins (6.3 fold), cell migration (5.3 fold), transcriptional regulation (4.8 fold), or negative regulation of physiological processes (4.6 fold). Modulated genes/markers are summarized in Table 3. In this embodiment, the invention leads to a prospective identification, quantification and characterization of the cardiovascular potential of any isolated cell for cardiovascular cell therapy by analyzing the expression pattern of one or more genes as listed in Table 3.

In a preferred embodiment, a panel of at least two such markers of Table 3 is used in the methods of the invention. In a further embodiment, the number of markers used in the panel can be 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 100 or more. In a further embodiment, all markers are listed in Table 3 are used in a single custom made microarray for the detection of MCP cells according to the methods of the invention.

The selection of genes used in the method of the invention can be made by using either the amount of up- or down-regulation of said genes or by their functional characteristics such as being surface markers, being involved in migration, morphogenesis and development, tube morphogenesis and vessel development, membrane association, cell migration, transcriptional regulation, involvement in pathways or negative regulation of physiological processes. Those characteristics are listed in Table 3. Preferred genes from the table for use in the methods of the invention are those genes that are up-regulated at least or more than 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 10, 5 fold, 11 fold, 11.5 fold, 12 fold, 12.5 fold, 13 fold, 13.5 fold, 14 fold, 14.5 fold, 15 fold, 15.5 fold, 16 fold, 16.5 fold, 17 fold, 17.5 fold, 18 fold, 18.5 fold, 19 fold, 19.5 fold, 20 fold, 20.5 fold, 21 fold, 21.5 fold, 22 fold, 22.5 fold, 23 fold, 23.5 fold, 24 fold, 24.5 fold, 25 fold, 30 fold, 35 fold, 40 fold, or 43 fold or are down-regulated at least or more than 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold.

The invention further provides for cardiovascular progenitor cells or cardiovascular cells obtained by the method of the invention that may be used for transplantation, cell therapy or gene therapy. Preferably, the invention provides a differentiated cell produced using methods of the invention that may be used for therapeutic purposes, such as in methods of restoring cardiac function in a subject suffering from a heart or vascular disease or condition.

The invention thus provides a method of treating or preventing a cardiovascular disease or condition. Cardiac disease is typically associated with decreased cardiac function and includes conditions such as, but not limited to, myocardial infarction, cardiac hypertrophy and cardiac arrhythmia. In this aspect of the invention, the method includes introducing an isolated differentiated cardiomyocyte cell of the invention and/or a cell capable of differentiating into a cardiomyocyte cell when treated using a method of the invention into cardiac tissue of a subject. The isolated cardiomyocyte cell is preferably transplanted into damaged cardiac tissue of a subject. More preferably, the method results in the restoration of cardiac function in person suffering from chronic or acute cardiac insufficiency.

In yet another aspect of the invention there is provided a method of repairing cardiac tissue, the method including introducing an isolated cardiomyocyte or cardiac progenitor cell of the invention and/or a cell capable of differentiating into a cardiomyocyte cell when treated using a method of the invention into damaged cardiac tissue of a subject.

It is preferred that the subject is suffering from a cardiac disease or condition. In the method of repairing cardiac tissue of the present invention, the isolated cardiomyocyte cell is preferably transplanted into damaged cardiac tissue of a subject. More preferably, the method results in the restoration of cardiac function in a subject.

The present invention preferably also provides a myocardial model for testing the ability of stem cells that have differentiated into cardiomyocytes to restore cardiac function.

The present invention preferably provides a myocardial model for testing the ability of stem cells that have differentiated into cardiomyocytes or cardiac progenitors using methods of the invention to restore cardiac function. In order to test the effectiveness of cardiomyocyte transplantation in vivo, it is important to have a reproducible animal model with a measurable parameter of cardiac function. The myocardial model of the present invention is preferably designed to assess the extent of cardiac repair following transplant of cardiomyocytes or suitable progenitors into a suitable host animal. More preferably, the host animal is an immunodeficient animal created as a model of cardiac muscle degeneration following infarct that is used as a universal acceptor of the differentiated cardiomyocytes. This animal can be any species including but not limited to murine, ovine, bovine, canine, porcine and any non-human primates. Parameters used to measure cardiac repair in these animals may include, but are not limited to, electrophysiological characteristic of heart tissue or various heart functions. For instance, contractile function may be assessed in terms of volume and pressure changes in a heart. Preferably, ventricular contractile function is assessed. Methods of assessing heart function and cardiac tissue characteristics would involve techniques also known to those skilled in the field.

The present invention also provides a model for the study of human cardiomyocytes in culture, comprising differentiated cardiomyocytes or cardiac progenitors of the invention. This model is useful in the development of cardiomyocyte transplantation therapies and for research purposes.

The present invention also provides a source of cardiovascular cells for tissue engineering, that can be used in the development of transplantation therapies and for research purposes.

The methods of the invention further allow a large production of cardiovascular cells, which is a substrate of choice for the large scale screening of new molecules, or identification of novel effects of known drugs in cardiovascular drug research.

The invention further provides for use of cardiovascular cells obtained by the methods as indicated above, for evaluating the cardiovascular effects of a drug on differentiated cardiac cells or for evaluating the cardiovascular effects of a drug during cardiovascular development.

The invention provides for an assay for assessing the pharmacology of a candidate drug comprising the steps of: a) differentiating stem cells into cardiovascular progenitor cells according to the method of the invention, b) subjecting said cells in vitro to said candidate drug, and c) analysing the behaviour of said cells in the presence and absence of said candidate drug.

In another embodiment, the invention further provides for use of cardiovascular cells obtained by the methods as indicated above for the preparation of a medicament for restoring cardiovascular functioning in a subject.

The invention further provides for use of a Mesp-1-expressing vector in the preparation of a medicament for restoring cardiovascular functions in a subject.

The present invention also provides a method of conducting in vitro drug metabolism studies comprising: (i) exposing a heart stem cell or cell population thereof according to the invention, to a test agent, and (ii) observing at least one change, if any, involving the test agent after a predetermined test period. Preferably, the at least one change includes a change in the structure, concentration, or both of the test agent.

The invention further provides for an assay for assessing the toxicity of an agent on heart or vascular cells, comprising the steps of: a) differentiating stem cells into cardiovascular progenitor cells according to the method of the invention, b) subjecting said cells in vitro to said agent, and c) analysing the toxic effect of said agent on the cells obtained in step a). So far, no method is available to generate specific cardiovascular cells. The present invention allows the generation of large source of cardiovascular cells that can be used to perform a large scale screening of drug toxicity or to study the molecular mechanism underlying cardiac toxicity of drugs and to identify new targets to prevent it. The present invention also provides a method of conducting in vitro toxicity testing comprising: exposing to a test agent a heart stem cell or cell population thereof according to the invention, and observing at least one effect, if any, of the test agent on the population of heart cells. Preferably, the at least one effect includes an effect on cell viability, cell function, or both.

The invention further provides for tools for molecular diagnosis in Congenital Heart Disease (CHD). Different congenital heart diseases are characterized by abnormal closure or malposition of cardiac structures following a migration or specification defect. The invention therefore further encompasses a method for studying genetic defects in Mesp1 during the onset of progression of cardiac malformation. The occurrence of mutation of in Mesp1 genomic region (coding sequence and promoter), and its target genes, in congenital heart diseases in which a cell migration or specification defect can be involved, can also be studied. The invention provides a large scale strategy that will allow the clinical detection of such condition.

The present invention also provides a method for enhancing the regeneration of an injured or diseased heart comprising administering into the liver an effective amount of a heart stem cell or cell population thereof according to the invention.

The present invention also provides a method of conducting testing for efficacious agents for treating heart infections comprising (i) infecting with an infectious agent of interest a heart stem cell or cell population thereof according to the invention to provide an infected population, (ii) exposing the infected population to a predetermined amount of test agent, and (iii) observing effects, if any, of the exposure on the infected population. In an embodiment, the infectious agent includes a microorganism. In another embodiment, the infectious agent includes one or more viruses, bacteria, fungi, or combinations thereof. In a particular embodiment, the observed effects include effects on viral replication of a viral infectious agent.

The present invention also provides a method for treating errors of gene expression comprising: (i) introducing into a heart stem cell or cell population thereof prepared according to the invention a functional copy of a gene to provide a transformed population; and (ii) introducing into a patient's heart, which patient is in need of the functional copy of the gene, at least a portion of the transformed population.

The present invention also provides a composition for treating errors of gene expression comprising a transformed heart stem cell or cell population thereof according to the invention into which a functional copy of a gene has been introduced.

The present invention also provides a pharmaceutical composition for treating errors of gene expression comprising a heart stem cell or cell population thereof prepared according to the invention into which a functional copy of a gene has been introduced and a pharmaceutically acceptable carrier.

The heart stem cells according to the invention are particularly useful in medicine, cardiology, inborn errors of hearty functioning or differentiation, transplantation, infectious diseases, heart failure. The heart stem cells according to the invention are particularly useful for (human) heart cell transplantation, the preparation of animal models of human heart cell transplantation, bioartificial hearts, in vitro heart cell lines and animal models of acquired human heart diseases or heart-function disorders, heart rhythm tests and heart cell directed gene therapy. The heart stem cell according to the invention can be further differentiated into cardiomyocytes or vascular cells.

In the present invention the term “stem cell” is preferably a human stem cell and is undifferentiated prior to culturing and is capable of undergoing differentiation. The stem cell may be selected from the group including, but not limited to, isolated embryonic stem (ES) cells e.g. human embryonic stem cells (hES), established embryonic stem cell lines (e.g. human), pluripotent stem cells, haematopoietic stem cells, totipotent stem cells, mesenchymal stem cells, neural stem cells, or adult stem cells. The stem cell is preferably a human embryonic stem (hES) cell which may be derived directly from an embryo or from a culture of embryonic stem cells. For example, the stem cell may be derived from a cell culture, such as human embryonic stem cells (hES) cells as disclosed in Reubinoff et al., (Nature Biotech. 16:399-404 2000). The stem cells may be derived from an embryonic cell line or embryonic tissue. The embryonic stem cells may be cells which have been cultured and maintained in an undifferentiated state. Such cells have been described in WO2000/027995, WO2001/042421, WO2001/098463 and WO2001/068815, the contents of which are incorporated herein by reference.

The stem cells suitable for use in the present methods may be derived from a patient's own tissue. This would enhance compatibility of differentiated tissue grafts derived from the stem cells with the patient. The stem cells may be first genetically modified prior to use through introduction of genes that may control their state of differentiation prior to, during or after their exposure to the factors that contribute to the promotion of cardiovascular differentiation of the stem cells. They may be genetically modified through introduction of vectors expressing factors that induced pluripotent states such as Oct4, Sox2, Nanog, or Klf4, or selectable marker under the control of a stem cell specific promoter such as Oct-4 or of genes that may be upregulated to induce differentiation such as Mesp1. The stem cells may be genetically modified at any stage with markers or gene so that the markers or genes are carried through to any stage of cultivation. The markers may be used to purify the differentiated or undifferentiated stem cell populations at any stage of cultivation.

It is expected that these culture conditions for improved or enhanced differentiation will be applicable at least to all stem cell lines from the same sources as those tested and suggest that these culture conditions for improved differentiation are applicable to all stem cell lines and stem cells in general. Furthermore, the fact that these differentiation conditions can be established without fetal calf serum, and thus without the potential presence of animal pathogens, increases the chance that these hES-derived embryonic germ layer derivatives such as ectoderm, mesoderm or endoderm, more preferably cardiomyocytes or cardiac mesoderm are suitable for transplantation in patients preferably with heart disease. Cardiomyocyte or cardiac mesoderm differentiation.

The terms “cardiac differentiation”, “cardiomyogenic differentiation”, “cardiomyogenesis” or “differentiating stem cells into cardiomyocytes” means the formation of cardiomyocytes from stem cells preferably from hES cells. Formation of cardiomyocytes is defined by the formation of contracting EBs, contracting seeded cells, immune cytological staining for cardiomyocyte specific marker, and expression of cardiomyocyte specific marker.

Culturing the stem cells can be done in the presence of a medium that is substantially free of xeno- and serum-components and thus comprises a clinically compliant medium, free of contaminants such as bacteria, virusses, growth factors, allergens, prions, etc. In the method of the invention, the stem cells can be transiently transformed with a plasmid encoding Mesp1 protein under the control of an inducible promotor or expression system.

### EXAMPLES

The invention is illustrated by the following non-limiting examples

### Materials and Methods

### Tetracycline Inducible Mesp1 ES Cell Line

The Mesp1 gene and protein are as identified by Saga et al., 1996, Development 122, 2769-2778, Genbank accession number NM_{—}018670 and Genpept accession number NP_{—}061140. Murine Mesp1 ORF was cloned by PCR using the IMAGE clone 4006663 as template with primers adding a 3Xflag sequence in its C-terminus part, followed by a double stop codon. The PCR product was inserted into a TOPO vector (Invitrogen, Carlsbad) for sequencing and then subcloned in the p2LoxGFP vector in place of the GFP using Sall and EcoRI restriction sites. We added to this P2LoxMesp1 vector an IRES-EGFP subcloned from pIRES2-EGFP vector (Clontech) using the EcoRI and NotI sites. The final vector p2LoxMesp1-IRES-GFP was co-electroporated with the pSalkCre vector in A2Lox cells (MI and MK, manuscript submitted). For DsRed constructs, DsRed ORF was PCR amplified from pIRES2-DsRedexpress vector (Clontech) and cloned in place of the GFP in the P2LoxGFP vector using SallSmal sites.

### ES Cells Culture and Differentiation

A2Lox ES cells were maintained on irradiated MEFs in DMEM (Gibco) supplemented with 15% ES-cells qualified FBS (Gibco), 0,1 mM non essentials amino acids (Gibco), 1 mM sodium-pyruvate (Gibco), 0,1 mM □-mercaptoethanol (Sigma), 100 U/ml Penicilline (Gibco), 100 □g/ml Streptomycin (Gibco) and 1000 U/ml LIF (ESGRO). For EB differentiation, ES cells were trypsinized and resuspended in the same medium without LIF, supplemented with ascorbic acid 50 μg/ml (Sigma), and plated for 20 min in gelatin coated Petri disches to allow MEFs to adhere. Non adherent cells were collected and plated in hanging drops at 1000 cells per 25 μl drop. Concentrated doxycycline (Sigma) was added to hanging drops at corresponding days to a final concentration of 1 μg/ml as previously described (Kyba et al., 2002). EBs were collected after four days of differentiation and then plated in gelatin-coated Petri dishes for further differentiation. Medium was replaced at days 5, 7 and 9 of differentiation. For conditioned medium experiments, Mesp1 expressing cells producing the conditioned medium were differentiated in hanging drops 24 hours in advance to medium receiving cells. Medium receiving cells were initially differentiated in hanging drops of 20 μl and received a supplemental 25 μl of centrifuged conditioned medium at day 2 and day 3 of differentiation. After cell replating, medium were changed every day with conditioned medium. For mixed Mesp1-IRESGFP/DsRed expressing cells experiments, cells were plated in hanging drops in an equivalent ratio.

### Reverse Transcription and Quantitative PCR

Total RNA extraction and Dnase treatment of samples were performed using Absolutely RNA-microprep kit (Stratagene), according manufacturer's recommendations. 1 μg of purified RNA was used to synthesize the first strand cDNA in a 50 μl final volume, using a SuperscriptII (Invitrogen) and random hexamers (Roche). Control of genomic contamination was performed for each sample by performing the same procedure with or without reverse transcriptase. qPCR analysis were performed with one-twentieth of the cDNA reaction as template, using a Quantifast SYBR Green mix (Qiagen) on a ABI Fast7500 Real-Time PCR system. All primers were designed using Lasergene 7.2 software (DNAStar Inc) and are presented in Table 1. Specificity of the primers was assessed by electrophoresis of the amplicons on agarose gels. Analysis of results was performed by using the qBase (Hellemans et al., 2007) and GraphPad Prism softwares.

### Flow Cytometry Analysis

Before day 4 of differentiation, EBs were dissociated by trypsinization. After day 4 of differentiation, EB were dissociated by a half an hour collagenase IV treatment (Gibco) followed by trypsinization. For intracellular stainings, the BD Cytofix-Cytoperm kit was used according to manufacturer's recommendations. Anti cardiac isoform of troponinT (Ab1) staining (NeoMarkers, Fremont, Calif.) was performed for half an hour in PBS-BSA1% at a final concentration of 1/100, and revealed with an anti-mouse-PE secondary antibody (BD Biosciences). BrdU-APC and 7AAD stainings were performed using a BrdU flow kit according to manufacturer's recommendations (BD Biosciences). Active caspase-3 was revealed using an anti-caspase3-PE antibody 1/50 (BD Biosciences). Analysis was performed using a fluorescence-activated cell sorter (FACSCalibur, Beckton Dickinson, Immunocytometry Systems) and data were analyzed using CellQuest Pro.

### Immunofluorescence Analysis

After differentiation in hanging drops, EBs were plated on gelatin coated coverslips and fixed in 4% paraformaldehyde for 20 minutes, washed three times and then stained in a solution containing 1% BSA, 0,2% Triton, and 5% normal Donkey serum in PBS. Antibodies used were the following: anti cardiac isoform of troponinT Ab1 (clone 13-11; mouse monoclonal; 1/100; NeoMarkers, Fremont, Calif.), MF20 (mouse monoclonal-supernatant; 1/25; Developmental Studies Hybridoma Bank), Mlc2v (mouse monoclonal; 1/25; Alexis Corp), Mlc2a (mouse monoclonal; 1/200; Synaptic Systems), VE-cadherin (clone 11D4.1; Rat monoclonal; 1/100; BD Biosciences), Smooth muscle actin (clone 1A4; 1/200; Sigma), Islet1 (clone 39.4D5; mouse monoclonal-supernatant; 1/10; Developmental Studies Hybridoma Bank), Nanog (Rabbit polyclonal; 1/1000; Abcam), FoxA2 (clone 4 C7; mouse monoclonal concentrated; 1/100; Developmental Studies Hybridoma Bank). Primary antibodies were revealed with appropriate RRx-coupled secondary antibodies from Jackson laboratories (1/400). Single images and mosaics were acquired on a Zeiss Axio Imager with a Zeiss Axiocam MRn camer and using the Axiovision Rel. 4.6 software.

### Western Blot

50 mg of protein were loaded in 10% SDS-PAGE gel and incubate with anti-M2 (1/1000; Sigma). The amount of loaded proteins was normalized by the incubation with anti-□actin (abcam, Cambridge, UK) antibody.

### Chromatin Immunoprecipitation Assay

After 36 hours of Dox induction, EBs were harvested and fixed in 1% formaldehyde for 10 min at room temperature and neutralized with 125 mM glycine for 5 min at 4° C. After 10 min of incubation in cells lysis buffer (25 mM Hepes-KOH pH7.9; 1.5 mM MgCl2; 10 mM KCI; 0,1% NP40; 1 mM DTT; 1% Protease inhibitors), cells were centrifuged 5 min at 5.000 rpm at 4° C. and the pellet was resuspended in 300 ml sonication buffer (50 mM Hepes-KOH pH7.9; 140 mM NaCl; 1 mM EDTA; 1% triton; 0,1% Na-Deoxycholate; 0,1% SDS; 1% Protease inhibitors). DNA was then sonicated using Bioruptor (Diagenode, Liége, Belgium) following 30 cycles of 30s ON and 30s OFF. The DNA—protein complexes were pre-cleared 2h00 at 4° C. with protein G coupled to magnetic beads (Milteniy Biotec, Utrecht, The Netherlands) and then immunoprecipitated at 4° C. overnight with the anti-Flag M5 (Sigma-aldrich, Bornem, Belgium) or with related isotype. Finally, these complexes were incubated 2h30 at 4° C. with protein G coupled to magnetic beads (Milteniy Biotec, Utrecht, The Netherlands). The DNA—protein complexes were washed 4 times with the sonication buffer, 10 times with low-salt wash buffer (0,1% SDS; 1% triton; 2 mM EDTA; 150 mM NaCL; 20 mM TrisHCl pH8; 1% protease inhibitors), 20 times with high-salt wash buffer (0,1% SDS; 1% triton; 2 mM EDTA; 500 mM NaCl; 20 mM TrisHCl pH8; 1% protease inhibitors) and finally the samples were eluted with 400 ml Elution buffer (50 mM TrisHCl pH8; 1 mM EDTA; 1% SDS). After an overnight decrosslinking at 65° C. with 200 mM NaCl and 25 mg/ml Ribonuclease A (Sigma-aldrich), immunoprecipitated and input DNA were purified using the Qiaquick PCR purification kit (Qiagen, Venlo, The Netherlands) and real-time PCR was performed with 0,8 ml of purified DNA using the fast Sybr green master mix and designed primers compiled in Table 2.

TABLE 1
Primers used for gene specific PCR

| | | |
|---|---|---|
| Gene | Sense sequence (5′->3′) | Antisense sequence (5′->3′) |

SEQ ID No. 1 to SEQ ID No. 70

| | | |
|---|---|---|
| β-Actin | ACCAACTGGGACGATATGGAGAAGA | TACGACCAGAGGCATACAGGGACAA |
| TATA-BP | TGTACCGCAGCTTCAAAATATTGTAT | AAATCAACGCAGTTGTCCGTG |
| Brachyury | CTCCAACCTATGCGGACAAT | CCCCTTCATACATCGGAGAA |
| Mesp1 (ORF-3′UTR) | TGTACGCAGAAACAGCATCC | TTGTCCCCTCCACTCTTCAG |
| Nkx2.5 | CTCCGATCCATCCCACTTTA | AGTGTGGAATCCGTCGAAAG |
| Gata4 | CCACGGGCCCTCCATCCAT | GGCCCCCACGTCCCAAGTC |
| Hand2 | CCCGCCGACACCAAACTCTC | CCCCCGGCTCACTGCTCTC |
| Mef2c | AGGCACCAGCGCAGGGAATG | CCACCGGGGTAGCCAATGACT |
| TroponinT2 (cTnT) | GCGGAAGAGTGGGAAGAGACAGAC | GCACGGGGCAAGGACACAAG |
| Hand1 | GGTCGGCAGGTCCTTCGTGTC | GTGCGGCGGGTGTGAGTGG |
| Tbx1 | CAGCCCCGATTCCATGTTGTCTAT | GGTTCCGGGGCCAGTCCTC |
| Tbx5 | CTACCCCGCGCCCACTCTCAT | TGCGGTCGGGGTCCAACACT |
| Tbx20 | ATCGCCGCGCTTATGTCCAG | CCCCGCCGCCAAACTCC |
| Islet1 | AGCAGCAACCCAACGACAAAACTA | GTATCTGGGAGCTGCGAGGACAT |
| αMHC | GCTGGGCTCCCTGGACATTGAC | CCTGGGCCTGGATTCTGGTGAT |
| Mlc2a | AAGGGAAGGGTCCCATCAACTTCA | AACAGTTGCTCTACCTCAGCAGGA |
| M1c2v | ACTTCACCGTGTTCCTCACGATGT | TCCGTGGGTAATGATGTGGACCAA |
| ANF | ACCCTGGGCTTCTTCCTCGTCTT | GCGGCCCCTGCTTCCTCA |
| Kcne1 | CTGGGCTTCTTCGGCTTCTTCAC | CTACGGCCGCCTGGTTTTCAAT |
| CD31 | AATGGCAACTGGAGCGAGCACT | GGAGAAGGCGAGGAGGGTTAGGT |
| Gata1 | GTGGCGGAGGGACAGGACAG | GATTCGACCCCCGCTTCTTTTT |
| Tie2 | GGAGCCGCGGACTGACTACGA | CCTGCGCCTTGGTGTTGACTC |
| Runx2 | AGGCCGCCGCACGACAAC | CGCTCCGGCCCACAAATCTC |
| Colla1 | GTCCCCCTGGCTCTGCTGGTT | TCGGGGCTGCGGATGTTCTC |
| Albumin1 | CTTCGTCTCCGGCTCTGCTTTTTC | CGTTTCTTTCGGGCTCTTGTTTTG |
| AFP | GAGAAATGGTCCGGCTGTGGTG | GGGGGAGGGGCATAGGTTTCA |
| TCF1 | CTCCAGCCACCACCATCCACAT | GCTGCGGCCCTCCTCACC |
| Pdx1 | ACCTAGGCGTCGCACAAGAAGAAA | GCTCGCCTGCTGGTCCGTATT |
| Sox17 | GCGGCGCAAGCAGGTGAAG | GGGGCCCATGTGCGGAGAC |
| Sox18 | GCGCAGCCCCGAATCAGG | CGAGGCCCGGGAGCAAGAG |
| Gata6 | GCCGCACCGCTGACTCCTG | ACGCGCTTCTGTGGCTTGATGA |
| K8 | TGGCGGGGCTGGTGTCG | CTGCCGGCGGAGGTTGTTG |
| K14 | GCCGCCCCTGGTGTGGAC | GTGCGCCGGAGCTCAGAAATC |
| K18 | TGGACTCCGCAAGGTGGTAGATGA | ATTTCGGCAGACTTGGTGGTGACA |
| β3-tubulin | CCAGTGCGGCAACCAGATAGG | AAAGGCGCCAGACCGAACACT |

SEQ ID No. 71 to SEQ ID No. 128

| | | |
|---|---|---|
| Nestin | CGGAGAGGGAGCAGCACCAA | GGCCTCCCCCACAGCATCCT |
| Sox1 | GCGCCCTCGGATCTCTGGTC | GCCCGCGCCCTGGTAGTG |
| Islet1 | AGCAGCAACCCAACGACAAAACTA | GTATCTGGGAGCTGCGAGGACAT |
| Myocardin | AGTGGGCCCAGCATTTTCAACATC | CCCTCCCCATTTTCCCCACTTC |
| FoxH1 | GGGGCCTCGCGACAACTCTC | CACTGCCTGGACCTGACGGATAAT |
| Foxc1 | CCGGCCCCTATGAGCGTGTA | CTTCTTGTCCGGGGCATTCTGG |
| Foxc2 | CCAGAACGCGCCAGAGAAGAAGAT | CCGCCGCCGCCGCAGGAAG |
| Oct4 | CCAATCAGCTTGGGCTAGAG | TGTCTACCTCCCTTGCCTTG |
| Nanog | GCCTCTCCTCGCCCTTCCTCT | CCACCGCTTGCACTTCATCCTT |
| Sox2 | ACCGGCGGCAACCAGAAGAA | CAAGCCTCCGGGAAGCGTGTA |
| Eras | TGCTGGGCGTCTTTGCTCTTGA | CCTCCTGGGCCCTCTGAATCTC |
| Id2 | GCCGCTGACCACCCTGAACAC | AGAACGACACCTGGGCAAGACGA |
| Foxa2 | CTGAAGCCCGAGCACCATTACG | ATCCAGGCCCGCTTTGTTCG |
| FGF8 | TCAGCCGCCGCCTCATCC | CAGCGCCGTGTAGTTGTTCTCCAG |
| GSC | ACGAGGGCCCCGGTTCTGTA | CACTTCTCGGCGTTTTCTGACTCC |
| Cer1 | CAGCAGATGGGAGGAAAGTGGAG | GGGCGAGCAGTGGGAGCAG |
| Noda1 | GGGCGGATGGGGCAGAAG | CCGGTGGGGTTGGTATCGTT |
| DII1 | CGCCTTCAGCAACCCCATCC | CTGTGCGGCCGCTACTGTGAAG |
| DII3 | GCACGCCATTCCCAGACGAGT | CCGGGGACAGGCACATTCAAA |
| DII4 | GCGGCATGCCTGGGAAGTATC | GGGCCGGAGCTGGGTGTCT |
| Notch1 | AGGGGGAGGTGGATGCTGACT | GCTGGCGCCCTGGTAGATGAAG |
| Notch4 | GGCTGCCCCCTGGTTTCATT | TCTTCAGGGCCCGAGCACAT |
| Hes6 | GCCAGGGGGTGCACTAAAGAAAG | GCCCGCCTCCCCTGGTC |
| Hey2 | CCGAAAGCGACCTGGACGAGAC | ACCCCCTGTAGCCTGGAGCATC |
| Wnt3a | CACCCGGGAGTCAGCCTTTGT | GCGCCCAGCCTCATTGTTGT |
| Wnt5a | CGGGAGGGCGAGCTGTCTACC | CCTACGGCCTGCTTCATTGTTGTG |
| Dkk1 | GCTGCCCCGGGAACTACTGC | GAGCCTTCTTGTCCTTTGGTGTGA |
| Ripply2 | CGGGTCCGAGGGCTTCTGG | GCCCCGTCCGCTTCTCTTTCT |
| Mesp2 | CGCCTGGCCATCCGCTACAT | CACCCCCAGGACACCCCACTACT |

TABLE 2
Primers used to measure enrichment in regions containing conserved Ebox
sites following ChIP: Positions of primers and conserved Ebox sites
are indicated relative to the ATG for each gene.

| HAND2 | 5′ | 3′ | | 5′ | 3′ |
|---|---|---|---|---|---|
| cHand2AF1 AGAGAAGGCCTCGGCGGTAAC | 1652 | 1672 | EBOX | 1249 | 1254 |
| cHand2AR1 TCTAAACAGAAAGGGGGCGAGAG | 1855 | 1833 | EBOX | 1314 | 1319 |
| | | | EBOXA | 1989 | 1994 |
| cHand2BF1 CCCGGGATTGGCGTGAGG | −985 | −968 | EBOXA | −1170 | −1165 |
| cHand2BR1 GAAGCGGCGAATGGACTCTCG | −784 | −804 | EBOXB | −1159 | −1154 |
| | | | NBOX | −562 | −557 |
| cHand2CF1 TCATAAAAACTAGAAAATAAGCTCCGAACA | −1780 | −1751 | EBOXB | −1975 | −1970 |
| cHand2CR1 GTTAGGATGACAACTTGCAGAGAACG | −1554 | −1579 | EBOCC + NBOCC | −1911 | −1906 |
| | | | EBOXA | −1836 | −1831 |
| cHand2DF1 AGGACATAATCATCTTACCCAGTCTACCTG | −2884 | −2855 | EBOXB | −3176 | −3171 |
| cHand2DR1 TTGATGGCAGAACAAAGTGACCTACATA | −2622 | −2649 | EBOX | −2807 | −2802 |
| cHand2EF1 CTCCAGCCACCTACAGAACGCTATCC | −3361 | −3336 | EBOXB | −3176 | −3171 |
| cHand2ER1 ACCACCAACAACAACAAAAAGTAGGGGTAT | −3113 | −3142 | EBOX | −2807 | −2802 |
| cHand2FF1 TGAGGAGTCTTCCCAATCAGTTTACTACCT | −4439 | −4410 | EBOXA + EBOXB | −4556 | −4551 |
| cHand2FR1 AAAGGCAAGGGCAATTTTGTATCTCGT | −4237 | −4263 | EBOXA | −4794 | −4789 |
| cHand2GF1 AATGCACACTCTGAAAAAGACTCATGACATTG | −6530 | −6499 | | | |
| cHand2GR1 AAAAACATTGGTAAAGTTAGCACTGGGATAC | −6354 | −6384 | | | |
| cHand2HF1 CAGAAAGTTCAGAGAATGGAAGGCTTGATATG | −7880 | −7849 | EBOX | −7694 | −7689 |
| cHand2HR1 GGCTTACCTTCCCAAGCTAGAGAAGAACCTAC | −7738 | −7769 | | | |
| cHand2IF1 AGAAAGGAAGCTGAAGAAACAACACTGGTG | −9535 | −9506 | NBOX | −9210 | −9205 |
| cHand2IR1 AAAGGGCTCTCAGCGTGGAGTTAGAC | −9406 | −9431 | EBOXB | −9096 | −9091 |
| cHand2JF1 CCCAACACCGAGGGGAGACTACC | −9822 | −9800 | EBOXA + EBOXB | −10145 | −10140 |
| cHand2JR1 GCTGTTAAACTGCTAGCATGATTTGTCGTAT | −9628 | −9658 | | | |
| cHand2KF1 AACAATGCCTTACGGTTATTTTCATAGTC | −10976 | −10948 | EBOX | −10894 | −10889 |
| cHand2KR1 TAATACTCCAAGGCATAGGAAATCGTAGTTAG | −10706 | −10737 | EBOXB | −10819 | −10814 |
| cHand2LF1 CTGTCAGTCAGCAGAAATAAAGAATCCTATTG | −11574 | −11543 | NBOX | −11655 | −11650 |
| cHand2LR1 AGAACACTTCTTTGGAATTCCTTTTTGGATAC | −11329 | −11360 | EBOXA | −11515 | −11510 |
| | | | EBOX | −11491 | −11486 |
| | | | EBOXB | −11019 | −11314 |
| cHand2MF1 TATCAGCCAGTTATTTCCAAGTATCAGAGTTA | −12974 | −12943 | EBOXB | −12875 | −12870 |
| cHand2MR1 CAGACATTTTTATTTCTTTTCCTCCGCTCATA | −12746 | −12777 | EBOXA | −12657 | −12652 |

SEQ ID No. 129 to SEQ ID No. 154

| | 5′ | 3′ | | 5′ | 3′ |
|---|---|---|---|---|---|
| MYOCARDIN | | | | | |
| cMyocAF AAAAATATTTGTGGCGCTGGTTT | 564 | 542 | EBOXB | 474 | 469 |
| cMyocAR TTATAGAGTGGGCGCGTTATCAGTTAC | 352 | 378 | EBOX | 452 | 447 |
| cMyocBF ACGCGGCCCCAGGAGTC | −410 | −426 | EBOXA-B | −547 | −552 |
| cMyocBR TCGGATACAGAGGGGAAGC | −724 | −706 | | | |
| cMyocCF ACAGGGTCCCACGTGCATCATTA | −1026 | −1048 | EBOXB | −1035 | −1040 |
| cMyocCR GGCCTCCACCTGTCATTGTCATTC | −1241 | −1218 | EBOXB + EBOCC | −1151 | −1156 |
| | | | EBOXA | −1230 | −1235 |
| cMyocCOF AGGGCCGGGCTTTTGCATCTAAC | 4800 | 4778 | | | |
| cMyocCOR TGTGCCTCCATGTCCAGTGATA | 4642 | 4663 | | | |
| NKX25 | | | | | |
| cN24AF ATGGTGGCGACGCAGGTTTCAC | 1 | −20 | EBOXA | −26 | −31 |
| cN25AR GGCCCAATGGCAGGCTGAATC | −212 | −192 | EBOXB | −26 | −31 |
| | | | NBOX | −151 | −156 |
| | | | EBOX | −318 | −323 |
| cN25BF ACGGGCAGTTCTGCGTCACCTAAT | −235 | −258 | NBOX | −151 | −156 |
| cN25BR TGGGATTTTCAGGCTAACGAGGAG | −377 | −354 | EBOX | −318 | −323 |
| cN25C AACGGTAATATTTCAGGCGTCAGC | −2191 | −2168 | EBOX | −2353 | −2358 |
| cN25C GCTGGCCCTGCGGATCG | −2124 | −2140 | EBOXA + B | −2353 | −2358 |
| cN25D AGCGGCCCCTTTGTTGATACAGTA | −3213 | −3190 | EBOXA | −3074 | −3079 |
| cN25D CTGCAATCAGCCGCGAAAAGTATA | −2910 | −2933 | | | |
| cN25E ACACGGGGAAAGCCCAGACTACG | −8951 | −8973 | EBOXB | −9133 | −9138 |
| cN25E GTGCACTCCGGAATTGTGAACG | −9281 | −9260 | | | |
| GATA 4 | | | | | |
| cG4AF GAAGCGAAGCGGCAGTCCTGAAG | −481 | −503 | EBOX | −588 | −593 |
| cG4AR GCTGTACTGGGCGTCCGTTGAACC | −638 | −615 | | | |
| cG4BF TCGTACTGTGCATCATGTGGGTGTCAA | −1190 | −1216 | EBOXB | −1203 | −1208 |
| cG4BR GAGAGGAAGGATTGGAATTAACAGGTG | −1520 | −1494 | | | |
| cG4CF CAAGCCTGAAAAACTGCAAGCACTA | −2561 | −2585 | EBOX | −2633 | −2638 |
| cG4CR ATGAACGTTGTAGGGTGATTTGAAAGA | −2846 | −2820 | | | |
| cG4DF CAGGGGAGCTTGAGCCGACTAC | −3335 | −3356 | NBOX | −3244 | −3249 |
| cG4DR TGCACTGCTGAATACTACCCACATACA | −3445 | −3419 | | | |
| cG4EF TTCCCAAAGCTCCCCCAACAGG | −4248 | −4269 | EBOXB + EBOCC | −4177 | −4182 |
| cG4ER GGAGGAAAGAGAAGGAGAATAAACACG | −4407 | −4351 | | | |
| cG4FF TCCAACAGCTGCCAGGCGACATT | −6174 | −6196 | EBOX | −6142 | −6147 |
| cG4FR GAAAGGAAAAGCGGTCTGGGATGGTC | −6405 | −6380 | | | |
| cG4GF CGAAGGGAGGGTGACGACGAC | −7193 | −7213 | EBOX | −7369 | −7374 |
| cG4GR CTGTGCGGCTTGTGAGTTGATTCC | −7415 | −7392 | | | |

| | | | |
|---|---|---|---|
| 5′ | 3′ | 5′ | 3′ |

SEQ ID No. 155 to SEQ ID No. 186

| | | | | | |
|---|---|---|---|---|---|
| FOXA2 | | | | | |
| cFoxA2AF ACCTGAAGCCCGAGCACCATTACG | 2092 | 2115 | EBOXA | 2656 | 2651 |
| cFoxA2AR GCATCCAGGCCCGCTTTGTTCG | 2311 | 2290 | | | |
| cFoxA2BF CGCGCTGCCAAACATAACTCTG | 350 | 329 | EBOX | 213 | 208 |
| cFoxA2BR GCCGCCTTTTCCGCCCTCCTTCTA | 182 | 205 | | | |
| cFoxA2CF CGGGGCGCTCGTAGACTT | −3031 | −3048 | EBOXA | −3269 | −3274 |
| cFoxA2CR TCGGCCCCAGGTGAGGTTTAG | −3316 | −3296 | | | |
| cFoxA2DF TTGTTTGGAAGTCTGGGTTTTAGTTAT | −5152 | −5178 | EBOX | −5221 | −5226 |
| cFoxA2DR TCCTCCTCACGGTTCTCTGTTGTTATT | −5460 | −5434 | EBOXA | −5240 | −5245 |
| | | | EBOX | −9922 | −9927 |
| | | | EBOX | −9950 | −9955 |
| | | | EBOX | −10143 | −10148 |
| GSC | | | | | |
| cGSCAF1 CTCCCGGACCCAAGCCTCACAACT | −2790 | −2813 | EBOXA | −2720 | −2725 |
| cGSCAR1 GGGCATGCGGGGCGACAAACAAT | −3108 | −3086 | EBOXB | −2987 | −2992 |
| | | | EBOX | −3031 | −3036 |
| | | | EBOX | −3043 | −3048 |
| | | | EBOXB | −3327 | −3332 |
| cGSCBF1 ATCAGCTTCTAACGTTTTCATTCA | −4432 | −4455 | EBOX | −4311 | −4316 |
| cGSCBR1 CTCTTTTAGCAGTTTTTCACAA | −4583 | −4562 | EBOX | −4370 | −4375 |
| | | | EBOX | −4553 | −4558 |
| | | | EBOX | −4584 | −4589 |
| | | | NBOX | −4612 | −4617 |
| | | | EBOXB | −4648 | −4653 |
| | | | EBOXB | −4727 | −4732 |
| | | | EBOX | −4815 | −4820 |
| | | | EBOX | −4889 | −4894 |
| | | | EBOX | −4908 | −4913 |
| cGSCCF1 TGAGAACGGGCCACTTACTAT | −4917 | −4937 | NBOX | −4612 | −4617 |
| cGSCCR1 TTTGGTGCGCCTTGGACTGATTTC | −5197 | −5174 | EBOXB | −4648 | −4653 |
| | | | EBOXB | −4727 | −4732 |
| | | | EBOX | −4815 | −4820 |
| | | | EBOX | −4889 | −4894 |
| | | | EBOX | −4908 | −4913 |
| | | | EBOX | −5208 | −5213 |
| | | | EBOX | −6231 | −6236 |
| SOX17 | | | | | |
| cSox17AF2 TCCAGGATTTATTTAAGATTGAGA | 2011 | 1988 | EBOXB | 1830 | 1825 |
| cSox17AR2 AACTACCCCGACATTTGAC | 1673 | 1691 | | | |
| cSox17BF2 GGGGCTGGCTCTGGTCGTCACT | 48 | 27 | EBOXA + EBOXB | −359 | −364 |
| cSox17BR2 AGGAGAGCAGCGGGAGGGTAGCA | −144 | −122 | | | |
| cSox17cF2 CAGACCCGCCAGCAGTGTGAG | −2955 | −2975 | EBOX | −3047 | −3052 |
| cSox17cR2 TTGGGGATGTGGCTTAGGCAGTAG | −3244 | −3221 | NBOX | −3110 | −3115 |
| | | | EBOX | −3313 | −3318 |
| cSox17DF2 GGGGGCTCATTCCGCACAC | −3984 | −4002 | EBOCC | −3944 | −3949 |
| cSox17DR2 GATGGGGTATGGGTTCTAAG | −4231 | −4212 | EBOXA | −4228 | −4233 |
| cSox17EF2 TGAGCCAGGACTAAATGAGAATAC | −7916 | −7939 | | | |
| cSox17ER2 TGGAGGGCAGGATGTGGGGTTTA | −8131 | −8109 | | | |

SEQ ID No. 187 to SEQ ID No. 210

| | | | | | |
|---|---|---|---|---|---|
| BRACHYURY | | | | | |
| cBrachAF CGCGCTGGAGCCCATTGT | −434 | −417 | EBOX | −492 | −487 |
| cBrachAR GACACCCTTTGAAGTACCGAGCAG | −331 | −354 | EBOXB | −220 | −215 |
| cBrachBF GGAGGGCGGGGGTGTCG (17) | −484 | −468 | EBOX | −492 | −487 |
| cBrachBR AGGCTGGGGGCCAACAATGG (20) | −404 | −423 | EBOXB | −220 | −215 |
| cBrachCF TTGACTTTCATGTCCTCCTCCACCGAGATT | −1742 | −1713 | EBOX | −1580 | −1575 |
| cBrachCR CCCTCCTCTGCCCTTTCCCACTGAATACTG | −1453 | −1482 | | | |
| DKK1 | | | | | |
| cDkk1AF GAATATGGGGAGAGAAGTGG | −2253 | −2234 | EBOX | −2611 | −2616 |
| cDkk1AR CAGCATACTACTAGCAATGTC | −2167 | −2187 | | | |
| cDkk1BF GCTTGTCTATCACGATGAGC | −3943 | −3924 | | | |
| cDkk1BR GCAAAGATTTCCCGTTCCTG | −3845 | −3864 | | | |
| RIPPLY 2 | | | | | |
| cRply2AF CGCATGCTGTTTTTCTCCCAGACC | −228 | −205 | EBOX | −156 | −151 |
| cRply2AR CTCGGCGCTCTCGGTGGTATCC | 23 | 2 | EBOCC | −142 | −137 |
| | | | EBOCC | −106 | −101 |
| cRply2BF TTCCCAACCACAAAAGTATCGTCT | −811 | −788 | EBOXB | −659 | −654 |
| cRply2BR TGTTACTTGAAGGGGATGGACAAT | −564 | −587 | | | |
| cRply2CF CAGCTCTGCTCAGTTCTGCGTCAG | −6806 | −6783 | EBOXA | −6844 | −6839 |
| cRply2CR TTTGAAACTCACTTGCCCAACCAA | −6546 | −6569 | EBOXA | −6773 | −6768 |
| | | | EBOX | −6648 | −6643 |
| | | | EBOX | −6539 | −6534 |
| cRply2COF GGGGACCATCAGCATCACG | −3460 | −3442 | | | |
| cRply2COR ATTCAGCGACTAAAGGGTTCTACG | −3268 | −3291 | | | |
| MESP1 | | | | | |
| cM1AF GGGCCTGAACCCTTTGAACC | −1123 | −1142 | EBOXA + EBOXB | −1188 | −1193 |
| cM1AR CCTGGCCATAGGTGCCTGACTTAC | −1236 | −1213 | | 762 | 762 |
| cM1BF CAGGAGAGGGAGGCTGTGAACGA | −4440 | −4462 | EBOXA | −4551 | −4556 |
| cM1BR CACAGGGGCAACAGTGGTAACAGA | −4660 | −4637 | EBOXB | −4620 | −4625 |
| | | | EBOXA | −4687 | −4692 |
| | | | NBOX | −4690 | −4695 |
| cM1CF TTTGGGGCCTGTGITTTGACAAGT | −4847 | −4870 | EBOXB | −4866 | −4871 |
| cM1CR GGCTGCAGAGTGGGTGGGAGTATG | −5095 | −5072 | | | |
| cM1DF ACTGGCCCTCCTCACACCTCTCG | −6180 | −6202 | EBOX | −6229 | −6234 |
| cM1DR TGGCCCAGGACCAGATAATCAGAT | −6284 | −6261 | EBOXA | −6283 | −6288 |
| SEQ ID No. 211 to SEQ ID No. 236 | | | | | |

### Example 1

### Transient Expression of Mesp1 Dramatically Accelerates and Increases Cardiac Differentiation from ES Cells

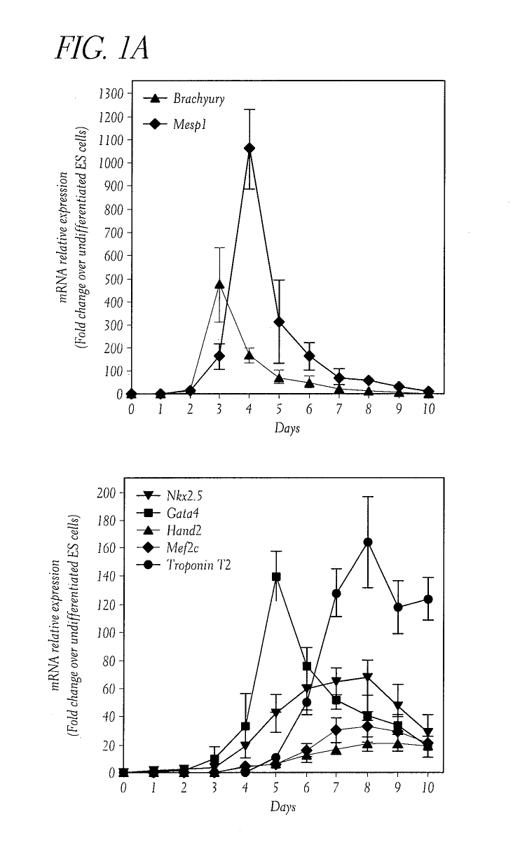

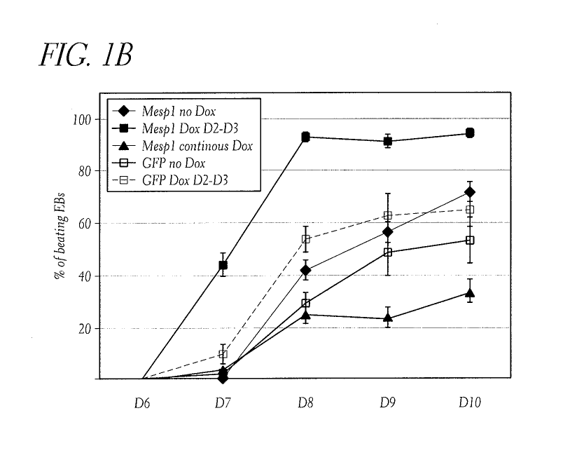

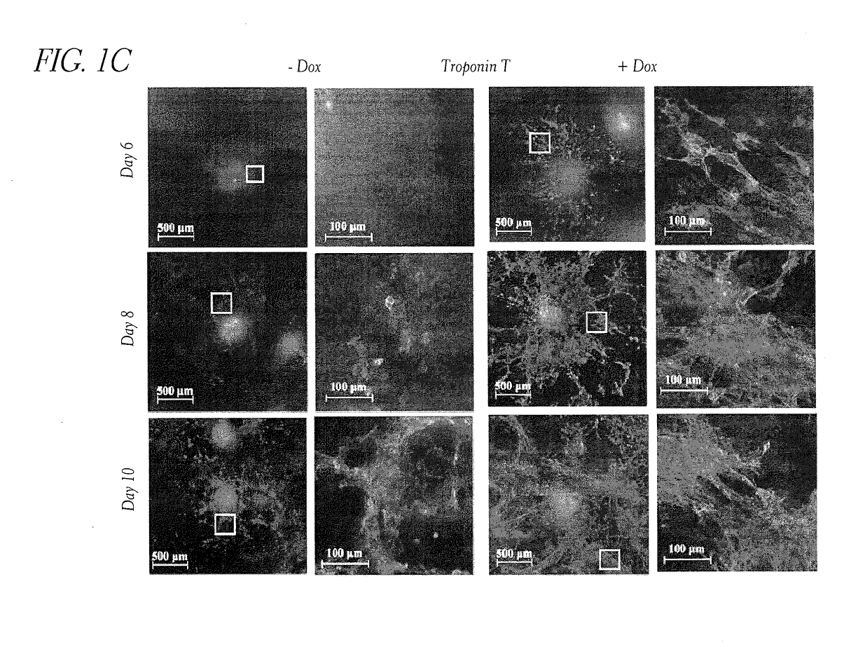

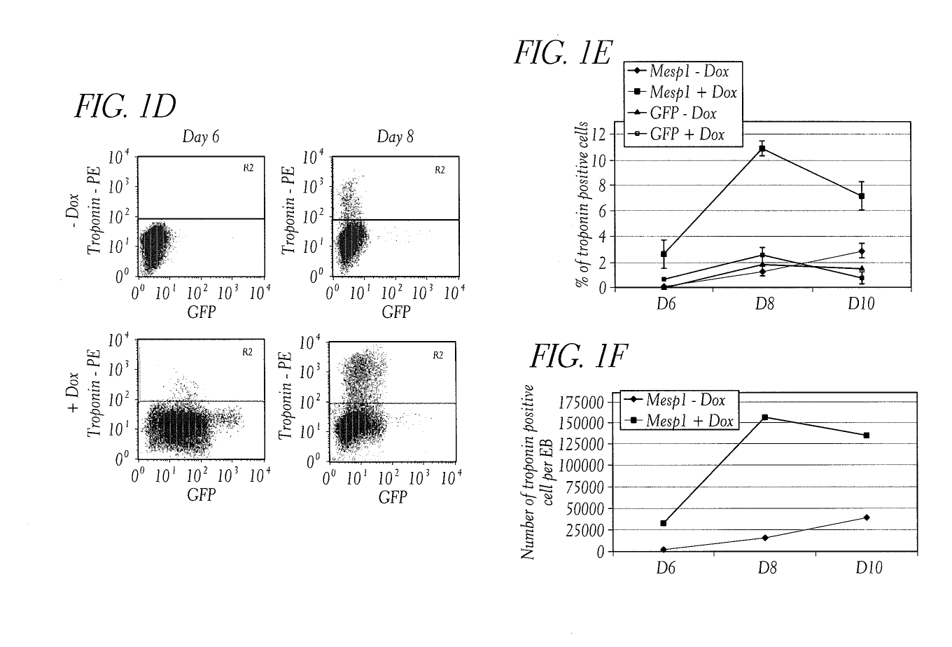

We first examined by RT-PCR the temporal expression of key transcription factors implicated in the transition from pluripotent ESC to cardiac terminal differentiation (FIG. 1A). When pluripotent ESCs are induced to differentiate, the temporal appearance of the key transcriptional factors implicated in mesoderm and cardiac commitment, is very similar to the temporal expression of these genes during embryonic development (Murry and Keller, 2008, Cell 132, 661-680). Genes regulating the specification of the primitive streak, such as Brachyury, are strongly and rapidly upregulated. Mesp1 began to be expressed soon after, peaks at day 4 (D4) and then was rapidly downregulated. Key cardiac transcription factors began to be expressed at D3-4, peaking around D6 while cardiac structural genes such as troponinT, began to be expressed at D5, peaked at D8 and their expression were maintained thereafter, in good accordance with the contractile phenotype of the cells observed upon microscopic inspection (FIG. 1B). To study the role of Mesp1 during cardiac cell fate specification, we generated a recombinant ES cell line, in which the expression of an epitope tagged version of Mesp1 followed by an IRES-GFP can be temporally and specifically induced upon doxyclin (Dox) addition (FIG. 8A) (Kyba et al., 2002, Cell 109, 29-37). To determine whether Mesp1 directly promotes cardiac specification, we induced the expression of Mesp1 from D2 to D3, one day earlier than its endogenous expression, and monitored the temporal appearance of beating EBs. In the absence of Dox, no expression of transgene was detectable by FACS, western blot or immunostaining analysis (FIGS. 8B-D). Upon Dox addition, Mesp1 is rapidly induced and 12h after Dox administration Mesp1 is clearly seen in the nucleus of ESCs and after 24 h about 80% of ESCs expressed Mesp1 (FIGS. 8B-D). The precocious expression of Mesp1 resulted in an acceleration of cardiac differentiation as demonstrated by the premature appearance of beating cells in the EB culture, which occurred at D7, a day earlier than in untreated cells or control GFP-inducible ESCs treated with Dox (FIG. 1B). A close observation of the EBs revealed an increased number of beating zones within EBs that has been stimulated with Mesp1. Immunofluorescence and FACS analysis demonstrated the precocious and increased expression of TroponinT, a cardiac specific marker, following Mesp1 induction (FIGS. 1C-F). Mesp1 stimulated cells generated four to five times more cardiac cells (FIGS. 1E and 1F), which represents one if not the greatest promotion of cardiac differentiation induced by a single factor.

### Example 2

### Mesp1 Specifically Promotes the Specification of Multipotent Cardio-Vascular Progenitors from Primitive Mesoderm

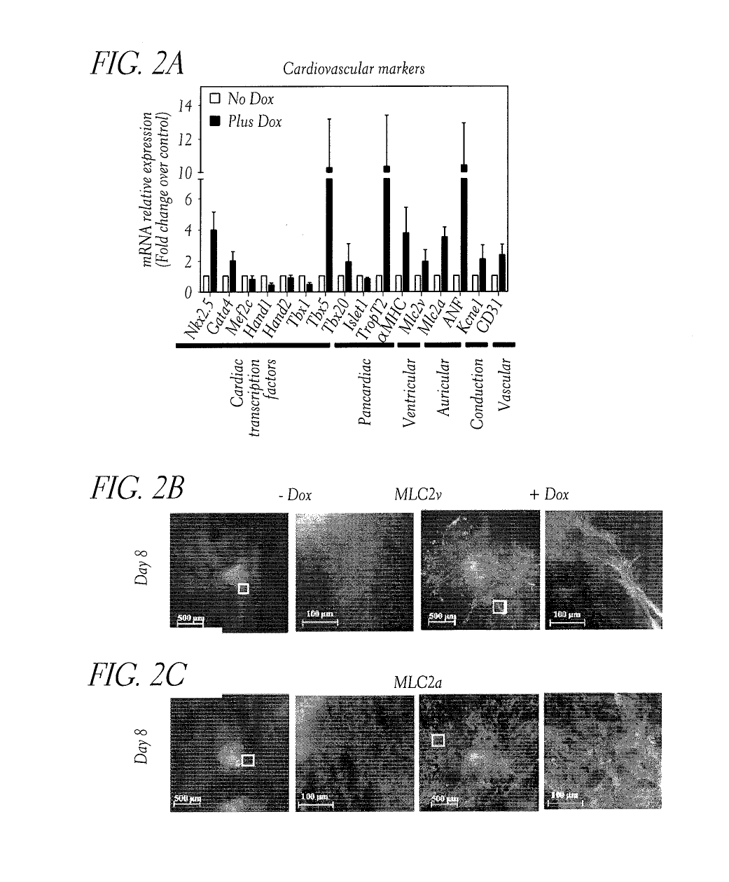

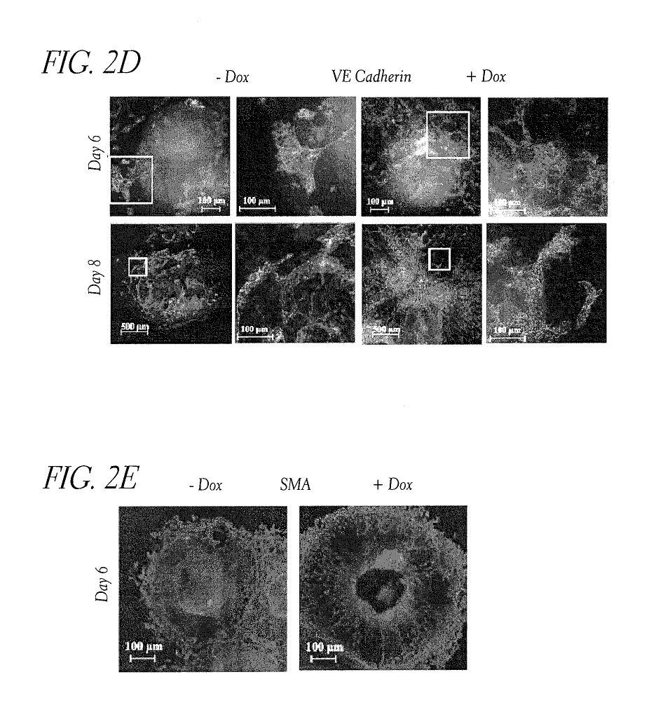

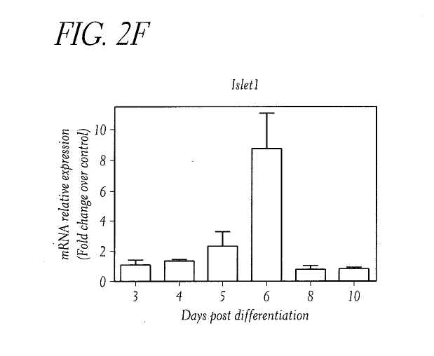

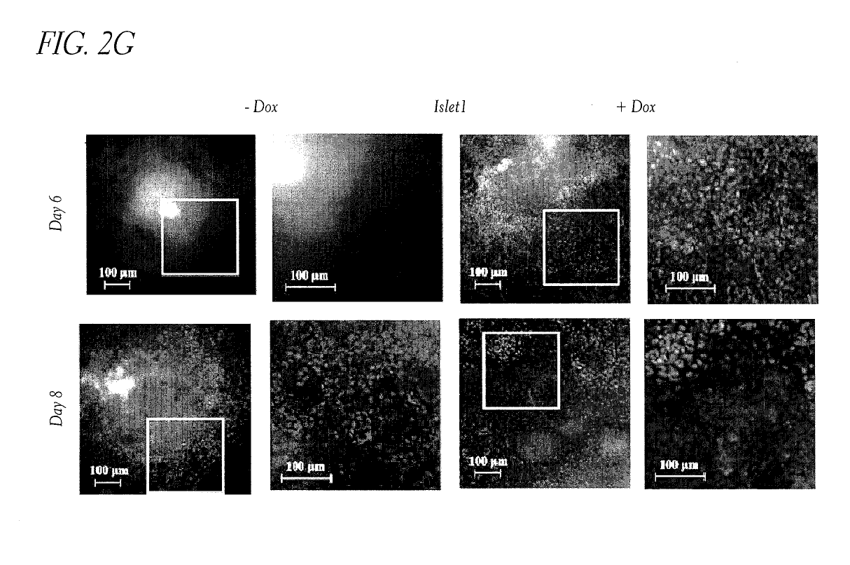

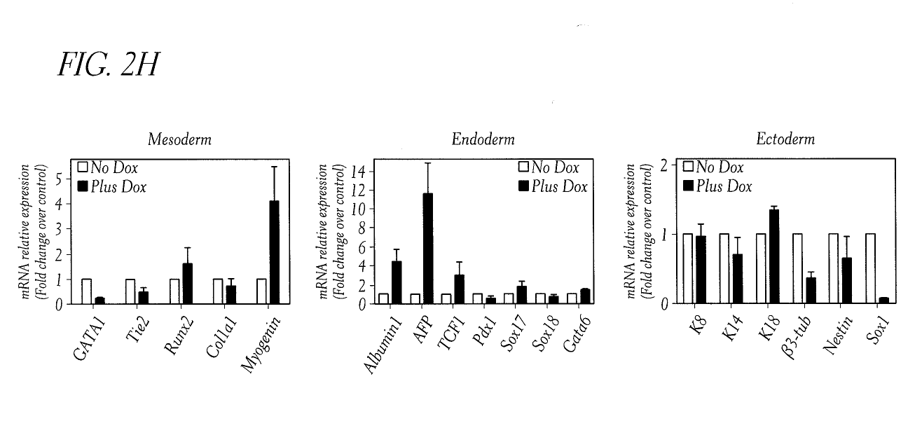

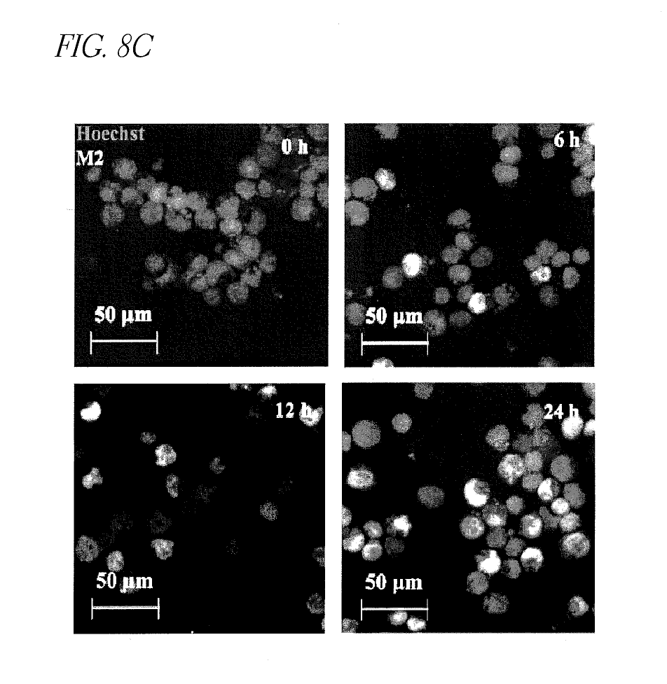

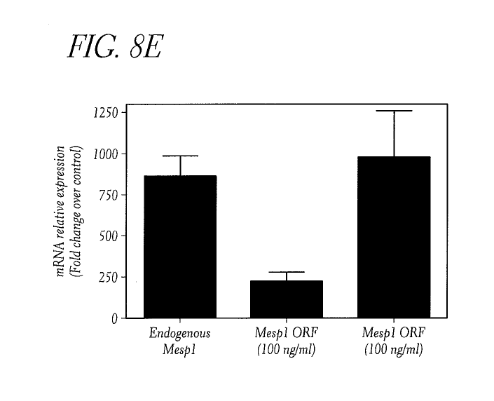

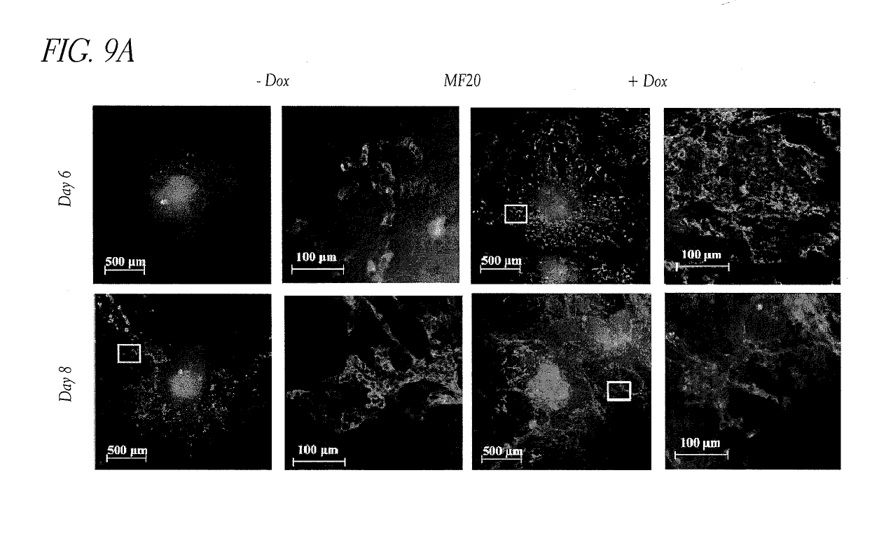

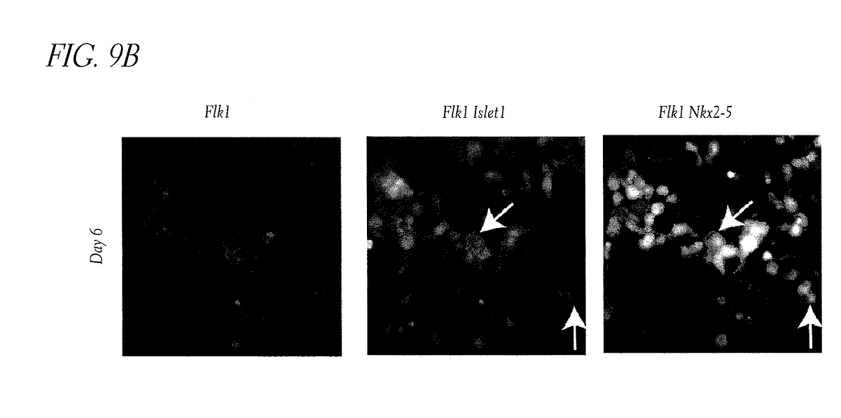

During embryonic development or ESC differentiation, cardiac cells are thought to arise from the differentiation of MCPs (Murry and Keller, 2008, Cell 132, 661-680). To determine whether Mesp1 promotes the specification of MCPs or whether its effect is restricted only to the promotion of cardiac differentiation, we analyzed by RT-PCR and immuno-staining the expression of markers specific for the different mature cardiovascular cell types. Mesp1 increased the expression of cardiac transcription factors such as Nkx2-5, Gata4, Tbx5 or Tbx20, pan-cardiac markers (troponinT, Mf20 and aMHC) (FIGS. 2A, 1C and 9), ventriclar markers such as Myosin Light Chain 2v (Mlc2v) (FIGS. 2A and 2B), atrial markers such as MLC2a or atrial natriuretic factor (FIGS. 2A and 2C), as well as markers of pace maker cells such as the potassium channel Kcne1 (FIG. 2A). In addition to promoting myocardial differentiation, Mesp1 also accelerated and promoted the differentiation of vascular cells as shown by the precocious and increased expression of CD31 and VE-Cadherin (FIGS. 2A and 2D), and smooth muscle cells as shown by expression of smooth muscle actin (SMA) (FIG. 2E). The similar promotion and acceleration in the differentiation of ESCs into different cardiac and vascular cell types induced by Mesp1 suggested that Mesp1 induced the specification of MCPs rather than only promotion of cardiac differentiation. Two distinct sources of cardiogenic mesoderm give rise to MCPs during embryonic development and ESC differentiation (Buckingham et al., 2005, Nat Rev Genet 6, 826-835; Laugwitz et al., 2008, Development 135, 193-205). The primary heart field originates from the anterior splanchnic mesoderm, and gives rise to the cardiac crescent, which contributes to the development of left ventricle and atria. The secondary heart field is derived from the pharyngeal mesoderm, anterior to the cardiac crescent, and is marked by the expression of Islet1 (Isl1), a transcription factor of the LIM9 homeo-domain family. Lineage tracing experiments using Isl1-CRE knock-in mice demonstrated that the secondary heart field gives rise to the outflow tract, the right ventricle and cells of atrial tissue (Buckingham et al., 2005, Nat Rev Genet 6, 826-835; Laugwitz et al., 2008, Development 135, 193-205). During ESC differentiation, Isl1 expression can also be used to isolate multipotent cardiovascular progenitors of the secondary heart field (Moretti et al., 2006, Cell 127, 1151-1165). To determine whether Mesp1 promotes the specification of MCPs from the primary and/or the secondary heart field, we monitored the expression of Isl1 following Mesp1 induction. During ESC differentiation, Isl1 protein is first detected around D6 and reaches its maximum at D8 (Moretti et al., 2006, Cell 127, 1151-1165). In Mesp1 stimulated cells, Isl1 mRNA expression was increased four days after Dox addition (D6), but this effect was only transient and Isl1 expression returned to the control level at D8 (FIG. 2F). Immunostaining of EBs revealed the precocious expression of Isl1 in Mesp1 stimulated cells, in which EBs at D6 contained as many Isl1 positive cells as did control cells two days later (FIG. 2G). Our results reveal that Mesp1 promotes the specification of MCPs from both primary and secondary heart fields. To determine whether Mesp1 promotes the fate of other cell types during ESC differentiation, we analyzed the expression of a panel of markers specific for the wide range of differentiated cells that are produced after 10 days of ESC differentiation (Keller, 2005, Genes Dev 19, 1129-1155). In addition to promoting an increase in the expression of cardiovascular markers, Mesp1 also increased the expression of hepatic markers such as Albumin, alpha-foeto protein or TCF1 (FIG. 2H), consistent with the known cellular non autonomous promoting effect of cardiac cells during liver development (Zaret, 2000, Mech Dev 92, 83-88). Mesp1 also promoted the expression of striated muscle cell markers such as MyoD or Myogenin (FIG. 2H), but this effect only appeared around D10 (FIG. 2H and data not shown), during the late stage of ESC differentiation, potentially related to the later expression and function of Mesp1 in the presomitic and somatic mesoderm (Saga, 1998, Mech Dev 75, 53-66; Takahashi et al., 2005, Development 132, 787-796). Expression of markers for other mesodermal derivatives such as hematopoieitic tissue (CD45, Gata1, Tie2), bone (runx2, Col2a1), endoderm (Pdx1, Sox17, Sox18, Hex) or neuro-ectodermal derivatives (Keratin 8, 14, 18, beta-Tubulin or Sox1), were unchanged or relatively decreased in Mesp1 stimulated cells (FIG. 2H). These results demonstrate that Mesp1 promotes very specifically cardiovascular progenitor specification during an early window of ESC differentiation.

### Example 3

### Mesp1 Specifies Multipotent Cardiovascular Progenitor Cell Fate by an Intrinsic and Cellular Autonomous Mechanism

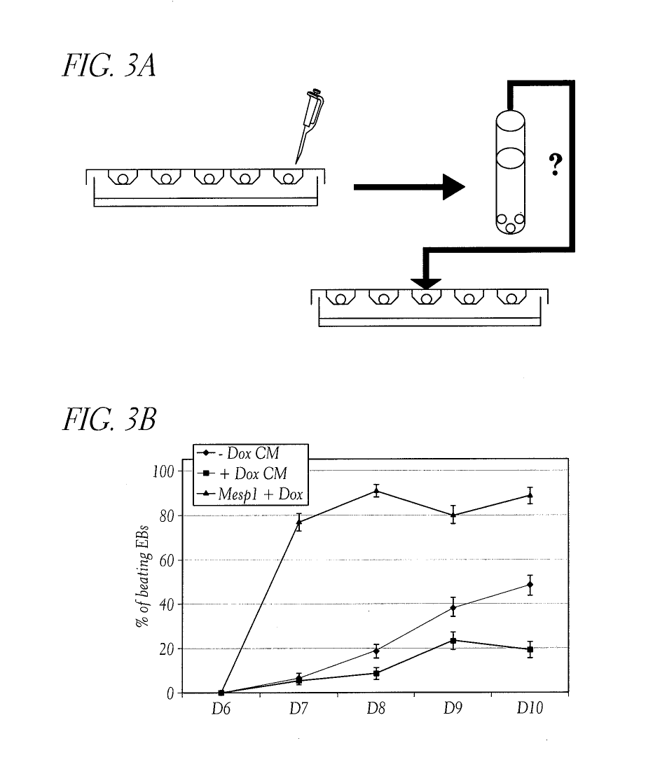

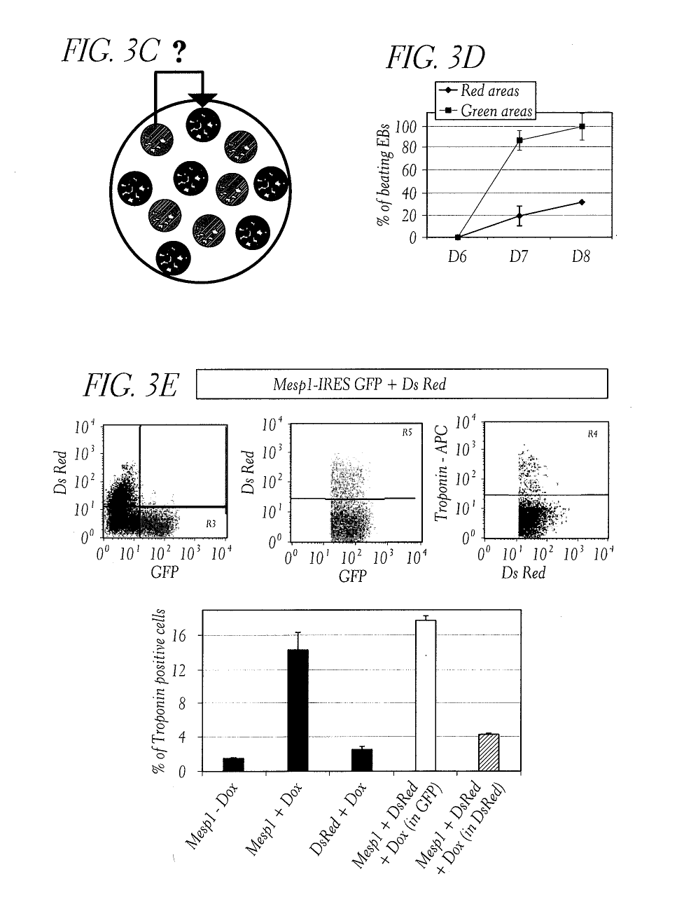

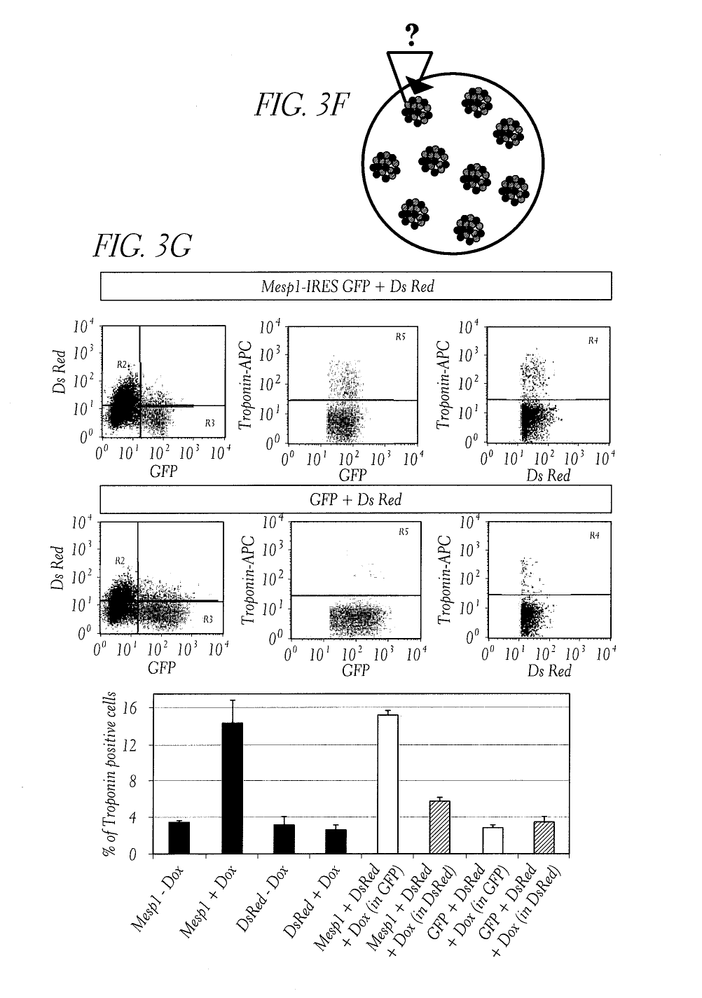

Cell fate can be specified by extrinsic cues, such as the secretion of soluble proteins, by intrinsic cues, such as the expression of transcription factors, or by a combination of both mechanisms. We used different cellular assays to determine the cellular mechanisms by which Mesp1 promotes MCP specification. We first determined whether the addition of conditioned media (CM) from Mesp1 stimulated cells to control cells could recapitulate the cardiac promoting effect of Mesp1 expression (FIG. 3A). Unlike Mesp1 stimulated cells, the daily addition of CM from Mesp1 stimulated cells did not promote or accelerate cardiac differentiation in control cells (FIG. 3B), indicating that Mesp1 does not promote cardiovascular specification by the secretion of soluble proteins. To validate these observations, we also cocultured Mesp1-IRES-GFP EBs with EBs that expressed the red fluorescent protein DsRed (FIG. 3C). While 80% EBs that expressed Mesp1 (GFP positive) showed beating zones at D7, only 20% of neigbhouring EBs that expressed DsRed presented signs of cardiac contraction at this stage (FIG. 3D). We quantified this effect by determining the expression of troponinT in cells expressing Mesp1 (GFP positive) and in control cells (DsRed) by FACS analysis (FIG. 3E). At day 8, 18% of Mesp1 stimulated cells expressed troponinT, whereas only 4% of the DsRed cells of the neighbored co-culture EBs, which is not significantly different than the control cells. These data showed the cardiac promotion induced by Mesp1 does no t involve the secretion of soluble molecules that act at long range. As a more rigorous test of an autonomous or non-autonomous mechanism, we generated chimeric EBs, in which ESCs conditionally expressing Mesp1 and DsRed are mixed together, to form EBs containing both cell types (FIG. 3A). These chimeric EBs were stimulated with Dox, and cardiac differentiation (troponinT+) was measured in the Mesp1 (GFP+) or control (DsRed+) cells by FACS. At D8, the percentage of troponinT positive cells was much higher in Mesp1 stimulated cells (15%) than in DsRed cells that had been in direct contact with Mesp1 stimulated cells (6%) or in DsRed cells than have been in direct contact with GFP cells (4%) (FIG. 3B). These results showed that Mesp1 promotes cardiac specification through an intrinsic and cell autonomous mechanism.

### Example 4

### Effect of Mesp1 on Cardiac Progenitor Cell Expansion

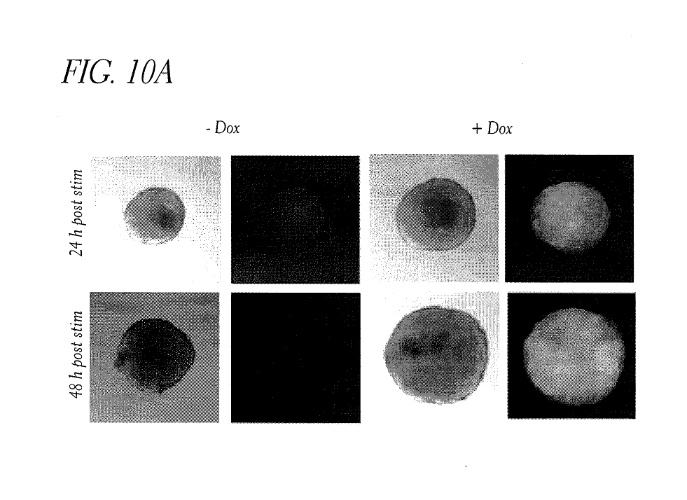

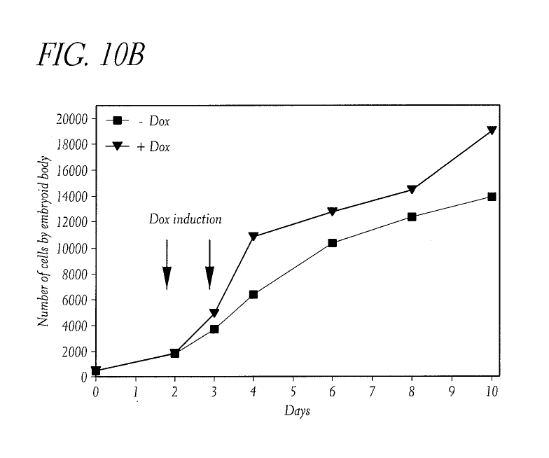

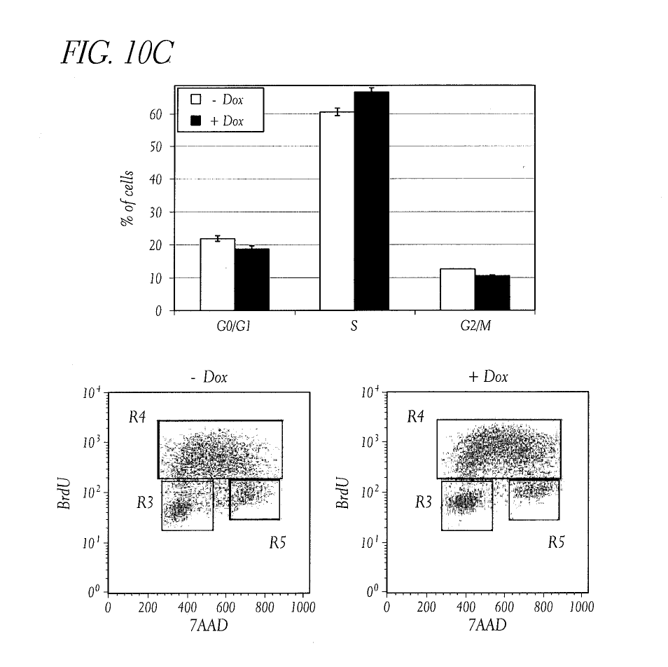

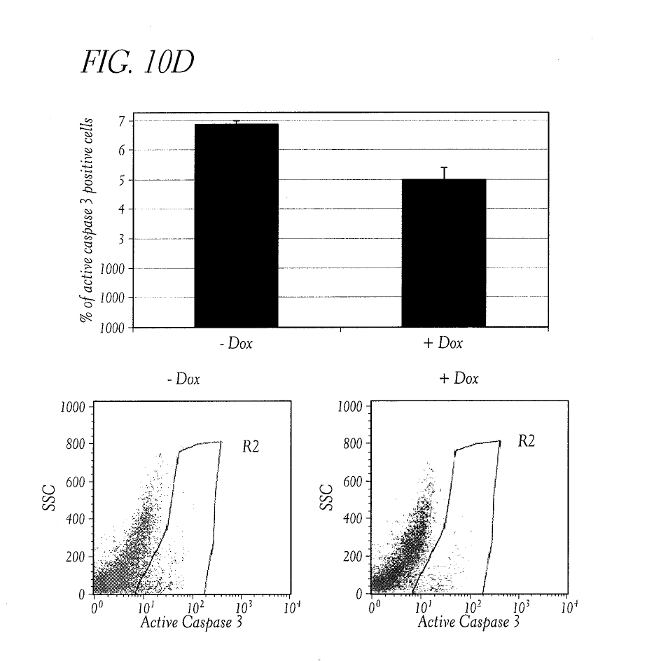

48 hours after Mesp1 induction, EBs were bigger and presented two times more cells than control EBs (FIGS. 10A and B). However, the growth advantage of Mesp1 stimulated cells was only transient since 3 days after Mesp1 induction, both Mesp1 stimulated and control cells grew at the same rate (FIG. 10B). To determine the cause of this transient growth advantage, we analyzed cell proliferation and apoptosis 48 h after Mesp1 induction. FACS analysis revealed that Mesp1 induction did not significantly modify the cell cycle profile of these cells (FIG. 10C) but apoptosis was significantly reduced in Mesp1 stimulated cells (FIG. 10D), suggesting that the transient growth advantage observed following Mesp1 induction, is related to a transient inhibition of apoptosis. The small and transient cell growth advantage observed in Mesp1 stimulated cells contrasted with the major increase in cardiovascular differentiation induced by Mesp1, strongly suggesting that Mesp1 promotes cardiovascular cell fate specification through an instructive rather than a selective mechanism.

### Example 5

### Mesp1 Directly Regulates the Expression of the Core Cardiac Transcriptional Machinery

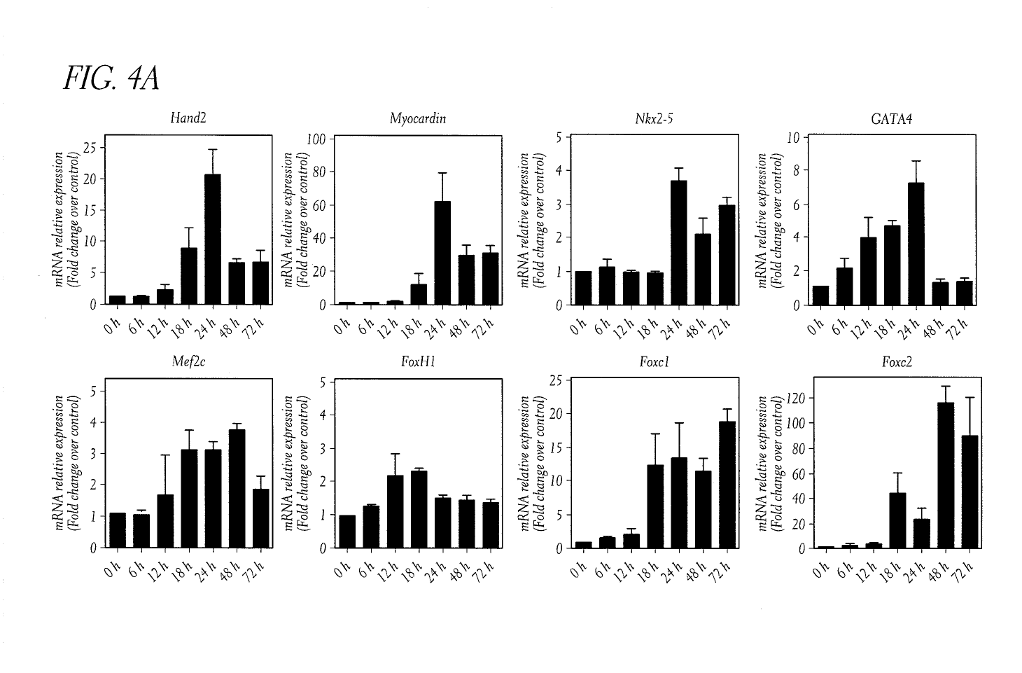

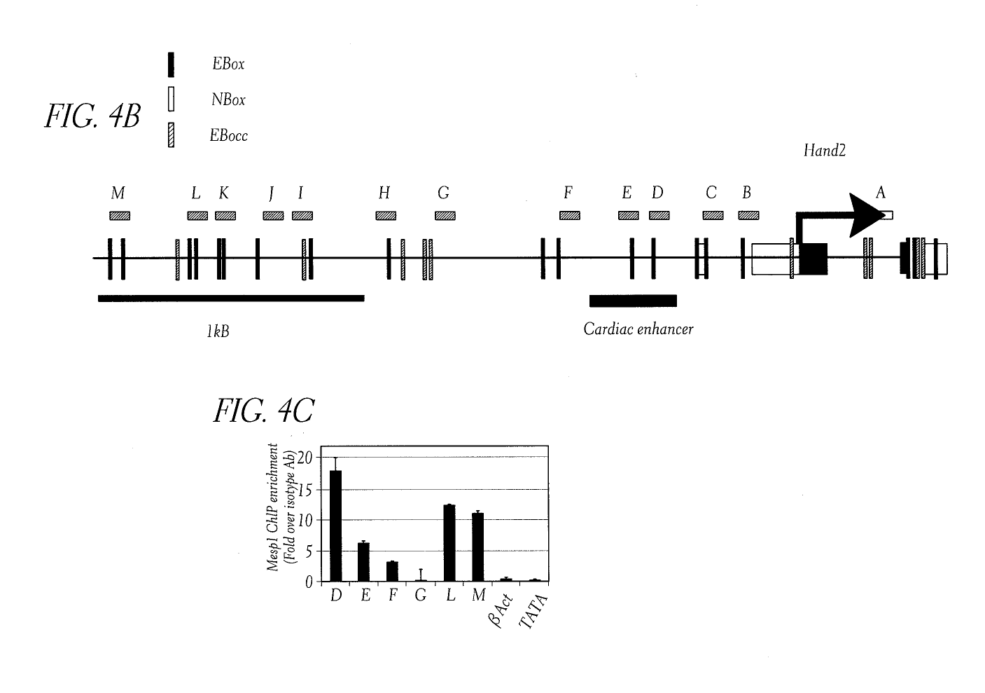

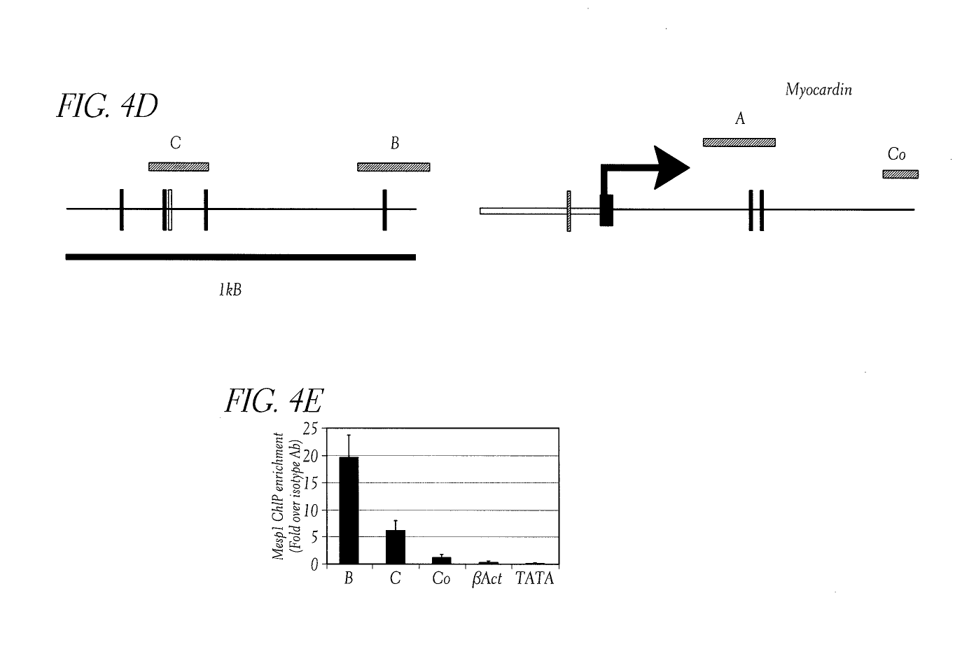

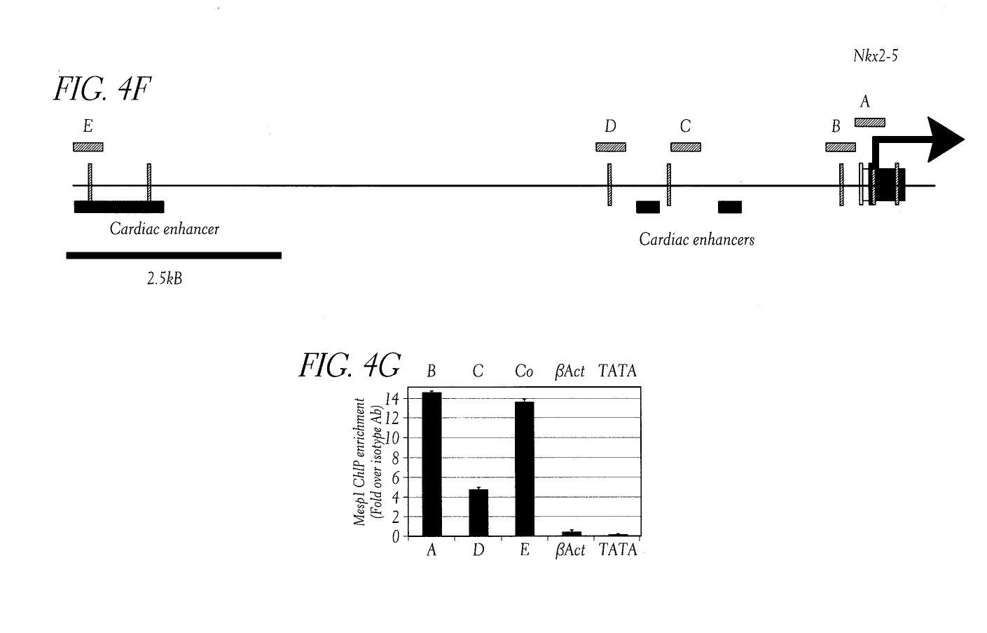

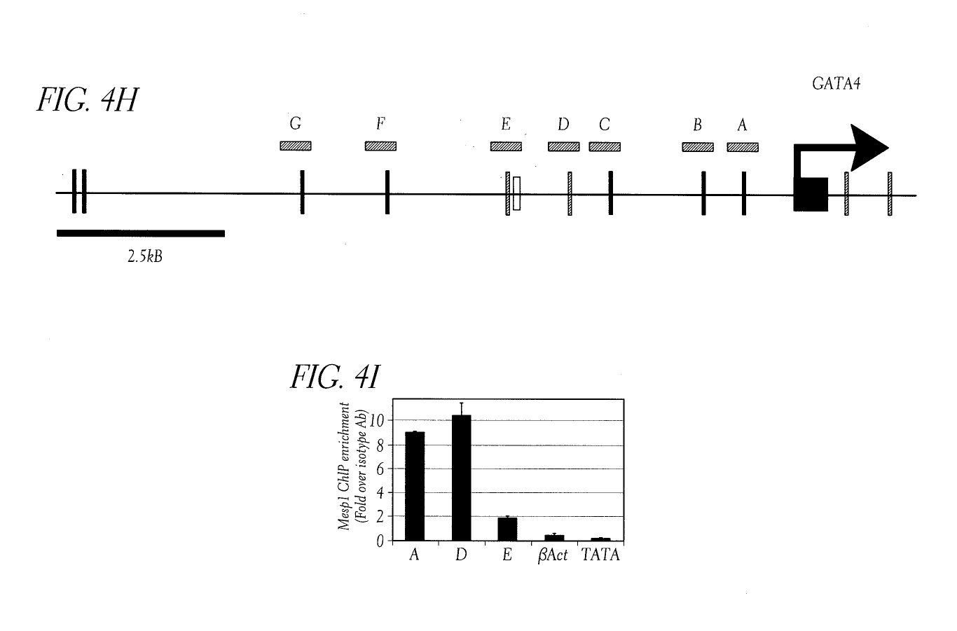

To uncover the molecular mechanisms by which Mesp1 induced cardiovascular specification, we performed a genome wide analysis of Mesp1 regulated genes. We determined which genes were regulated upon Mesp1 induction, by at least 1.5 fold in two separate experiments (Table 3). Surprisingly, Mesp1 regulated only a discrete set of genes (586 of 45101 probes), corresponding to 1.3% of the murine genome. Among the 423 unique annotated genes that were rapidly modulated upon Mesp1 induction, 276 were upregulated whereas 148 were downregulated, suggesting that Mesp1 exerts its function by both positive and negative regulation of gene expression. A functional annotation clustering of Mesp1 regulated genes revealed that Mesp1 preferentially regulated genes implicated in morphogenesis and development (enrichment score=16.1 fold), tube morphogenesis and vessel development (7 fold), membrane proteins (6.3 fold), cell migration (5.3 fold), transcriptional regulation (4.8 fold), or negative regulation of physiological process (4.6 fold). This analysis reveals that Mesp1 preferentially regulates the expression of genes controlling cardiovascular development and transcriptional regulation. Cardiac morphogenesis and differentiation are governed by an evolutionarily conserved set of transcriptional factors, which regulate the expression of genes implicated in morphogenesis and patterning during heart development, as well as the genes required for cardiac terminal differentiation (Olson, 2006, Science 313, 1922-1927). Our microarray analysis revealed that Mesp1 induced the rapid upregulation of many, if not all genes, belonging to the core cardiac transcriptional machinery: Hand2 was upregulated by 6,7 fold, Gata4 by 2.4 fold, Gata6 by 1.8 fold, Tbx20 by 1.9 fold, or Myocardin by 3.7 fold. (Table 3). In addition to these genes, we found that Nkx2-5 and Mef2c, although not listed in our microarray analysis, due to their low levels of expression, were also upregulated in our microarray and confirmed by RT-PCR (FIG. 4A). Other genes playing important role during cardiovascular development, such as Hey2 or Foxc1, were also rapidly upregulated following Mesp1 induction (Table 3) (Fischer et al., 2004, Genes Dev 18, 901-911; Kume et al., 2001, Genes Dev 15, 2470-2482). To study in more detail how Mesp1 regulates the expression of these key cardiac transcriptions factors, we investigated, by RT-PCR analysis, the kinetics of their upregulation following Mesp1 expression. As early as 18 h following Dox addition, 6 h only after the appearance of Mesp1 in the nucleus of ESCs, expression of Hand2, Myocardin, Gata4, FoxH1 and FoxC1 were already upregulated by 2 to 15 fold (FIG. 4A). For most of these genes, the maximum increase in gene expression occurred 24 hours following Mesp1 induction, although a sustained increase in the expression of Hand2, Myocardin, Nkx2-5, Mef2c or FoxC1 was still observed after 48 h or 72 h of Mesp1 stimulation (FIG. 4A). The very rapid upregulation of these genes by Mesp1 strongly suggests they are direct Mesp1 target genes. Other genes, such as Isl1 (FIG. 2F), presented their maximal expression only 96 h after Mesp1, suggesting an indirect mode of regulation of these genes by Mesp1.

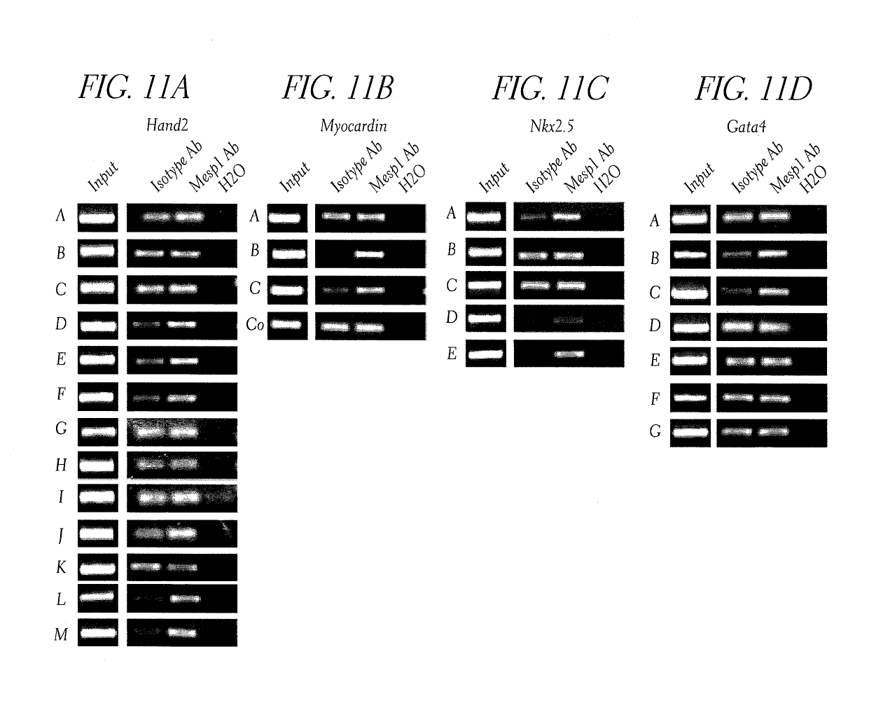

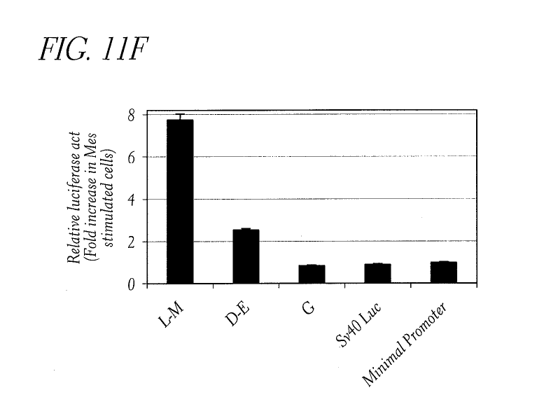

To determine whether these cardiac transcription factors are direct Mesp1 target genes, we performed chromatin immuno-precipitation (ChIP) analysis following Mesp1 induction and determined by PCR analysis, whether ChIP using anti-Mesp1 antibody (Ab), enriched for DNA fragments containing conserved putative Mesp1 binding sites (EBox). Quantitative PCR analyses were performed for DNA fragments showing different amounts of PCR products following ChiP using anti-Mesp1 and isotype control antibodies (FIGS. 11-14). Hand2 gene contains many conserved EBox sites in several conserved regions of its promoter (FIG. 4B). Our ChIP experiments demonstrated that multiple regions within the Hand2 promoter were enriched using Mesp1 Ab (FIGS. 4C and 11B). The strongest enrichment (about 20 fold) was found within a genomic region located 2.8 kB upstream of the ATG, a genomic region corresponding to the previously identified cardiac enhancer of Hand2 (McFadden et al., 2000, Development 127, 5331-5341). We identified other DNA regions also enriched following Mesp1 ChIP (FIGS. 4B, 4C and 11B). Several other regions containing a cluster of conserved EBox sites within Hand2 promoter were not significantly enriched by Mesp1 ChIP (FIGS. 4C and 11B), suggesting that Mesp1 binds directly and specifically to different promoter regions of Hand2, encompassing the previously identified cardiac enhancer (McFadden et al., 2000, Development 127, 5331-5341). To validate these results using another assay, we tested the ability of different Hand2 enhancers to promote transactivation of a reporter construct by Mesp1. Mesp1 stimulated the expression of reporter constructs containing the two distal enhancers and the more proximal cardiac enhancer, while no stimulation was observed in reporters without enhancer or containing a Hand2 enhancer without conserved EBox (FIG. 11F). We identified one DNA region within the proximal promoter of myocardin containing a conserved EBox site, that was strongly enriched by ChIP using Mesp1 Ab (about 20 fold) (FIGS. 4D, 4E and 11C). The Nkx2-5 promoter contained at least three regions enriched for Mesp1 binding (FIGS. 4F, 4G and 11D). Two of them are located in genomic regions previously identified as enhancers that promote Nkx2-5 expression in the cardiac crescent and heart tube (Schwartz and Olson, 1999, Development 126, 4187-4192). Gata4 contained two genomic regions located in the proximal promoter strongly enriched following Mesp1 ChIP whereas other regions containing conserved EBox sites and located more upstream were not enriched by Mesp1 Ab (FIGS. 4H, 4I and 11E).

### Example 6

### Mesp-1 Represses the Expression of Genes Regulating Pluripotency, Early Mesoderm and Endoderm Cell Fates

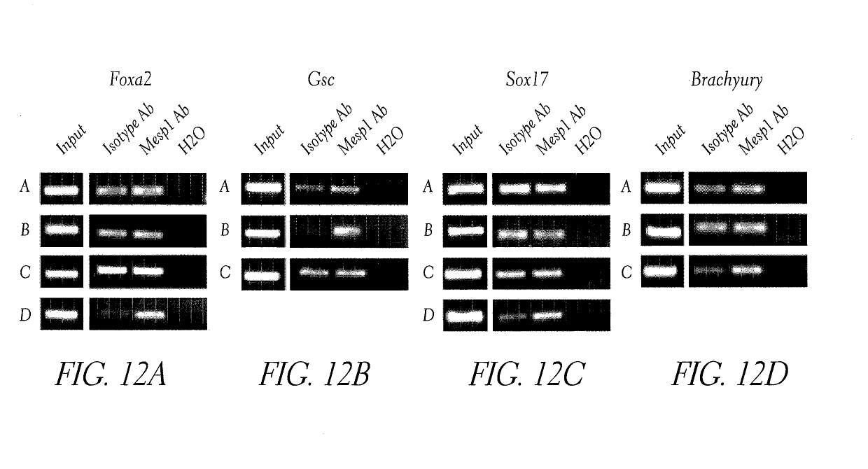

The micro-array analyses indicated that Mesp1 rapidly repressed the expression of several genes implicated in the maintenance of pluripotency such as Id2 and Eras (Table 3) (Takahashi et al., 2003, Nature 423, 541-545; Ying et al., 2003, Cell 115, 281-292). We expanded our analysis by examining the expression of other key regulators of pluripotency by RT-PCR (Jaenisch and Young, 2008, Cell 132, 567-582). Expression of Nanog, Oct4, Sox2 were also downregulated upon Mesp1 induction but less rapidly than Eras or Id2, suggesting they do not represent direct Mesp1 target genes (FIGS. 5A and 5B). Using immunostaining, we found that Nanog disappears more rapidly, during ESCs differentiation, in Mesp1 stimulated cells compared to control cells (FIG. 5C). During early gastrulation, specification of the primitive streak to mesoderm and endoderm cell fate is tightly regulated temporally and spatially by specific transcription factors such as Brachyury, Sox17, Foxa2 and also by different extrinsic factors such as Wnts or Nodal (Tam et al., 2003, Curr Opin Genet Dev 13, 393-400; Tam end Loebel 2007, Nat Rev Genet 8, 368-381). Specification of the PS to mesoderm and endoderm cell fate is tightly regulated temporally and spatially by specific transcription factors such as Brachyury, Sox17, Foxa2 but also by different extrinsic factors such as Wnts or Nodal (Tam et al., 2003, Curr Opin Genet Dev 13, 393-400; Tam and Loebel, 2007, Nat Rev Genet 8, 368-381). The formation of cells of the primitive streak also occurs during ESC differentiation, and gives rise to either mesoderm or endoderm cells (Murry and Keller, 2008, Proc Natl Acad Sci USA 103, 19812-19817). Our micro-array analysis showed that Brachyury (T) and FGF8, which are expressed throughout the primitive streak, as well as Foxa2 and Sox17, which are expressed in the anterior primitive streak and control the specification of definitive endoderm lineages, were among the five most downregulated genes following Mesp1 induction (Table 3). Several other genes important for early mesoderm and endoderm specification, such as Nodal, Goosecoid, Cerberus, Follistatin, and FoxD3 (Tam and Loebel, 2007, Nat Rev Genet 8, 368-381), were also downregulated following Mesp1 induction suggesting that Mesp1 selectively represses genes implicated in the specification of other early mesoderm and endoderm cell fates (Table3). We used RT-PCR to investigate in more detail the kinetics of their transcriptional repression by Mesp1. Brachyury, Sox17, FGF8, Foxa2, Gsc, and Cer1 were rapidly downregulated following Mesp1 expression as soon as 12 h after Dox addition, and reached their maximal downregulation 24 h after Dox addition (FIG. 5D). Among these genes, Foxa2 was the most downregulated. Using immunostaining, we demonstrated that Foxa2 expression was indeed strongly down-regulated in cells expressing Mesp1 (FIG. 5E). To determine whether Mesp1 directly controls the expression of these genes, we performed ChIP experiments following Mesp1 induction and determined by PCR analysis whether immunoprecipitated fragments contained conserved EBoxes. Our ChIP experiments revealed that Mesp1 IP enriched by about 20 fold DNA fragments containing one conserved EBox site located 5 kB upstream of the ATG of Foxa2 (FIGS. 5F, G and 12A). Our ChIP experiments showed that DNA fragments located 4.5 kB upstream of the ATG of Gsc and containing a cluster of 10 conserved EBox sites were enriched by 20 fold using anti-Mesp1 antibody (FIGS. 5H, 5I and 12B). Our ChIP experiments revealed that Mesp1 IP enriched by about 10 fold DNA fragments located 4 kB upstream of the ATG of Sox17 (FIGS. 5J, K and 12C). Mesp1 ChiP enriched by 5 fold DNA fragments located 1.5 kB upstream of the ATG of Brachyury (FIGS. 5M and 12D). The specific binding of Mesp1 to regions of genomic DNA located in Sox17, Gsc, Foxa2 and Brachyury revealed by our ChIP experiments, together with their very rapid downregulation following Mesp1 induction, strongly suggest that Mesp1 directly controls the repression of genes involved in the specification of the other cell types that arise during the early stages of gastrulation.

### Example 7

### Mesp1 Directly Regulates Multiple Components of the Canonical Notch and Wnt Signaling Pathways and Prime these Pathways Toward Cardiac Commitment

Wnt and Notch signaling pathways are well known to regulate different aspects of cardiovascular differentiation from progenitor specification to cardiac and vascular cell terminal differentiation (Cohen et al., 2008, Development 135, 789-798; Gridley, 2007, Development 134, 2709-2718).

Our microarray analysis revealed that many components of the Wnt and Notch pathways were rapidly upregulated following Mesp1 expression (Table 3). All three delta ligands, three of the four Notch receptors, and two well known downstream target genes of the Notch pathway (Hey2, Hes6) were upregulated following Mesp1 induction (Table 3). The promotion of Notch ligands and Notch target gene expression mediated by Mesp1 was relatively transient, peaked after 24 h, and decreased to the basal level thereafter, whereas the increase in Notch1 expression was more sustained. To investigate whether Notch signaling was necessary for the cardiovascular specification induced by Mesp1, we treated ESCs with N-[N-(3,5-Difluorophenacetyl)-L-alanyl]S-phenylglycine t-butyl ester (DAPT), a gamma-secretase inhibitor, that prevents Notch activation (Geling et al., 2002, EMBO Rep 3, 688-694), while at the same time inducing Mesp1 expression. DAPT did not profoundly reduce the number of beating EBs after 8 days of differentiation in control or Mesp1 stimulated cells. However, immunostaining analysis showed that DAPT treatment from D2 to D4 caused a decrease in the total number of troponinT and VE-Cadherin positive cells in both control and Mesp1 stimulated cells, suggesting that Notch influences MCPs specification and/or early cardiovascular lineage commitment.

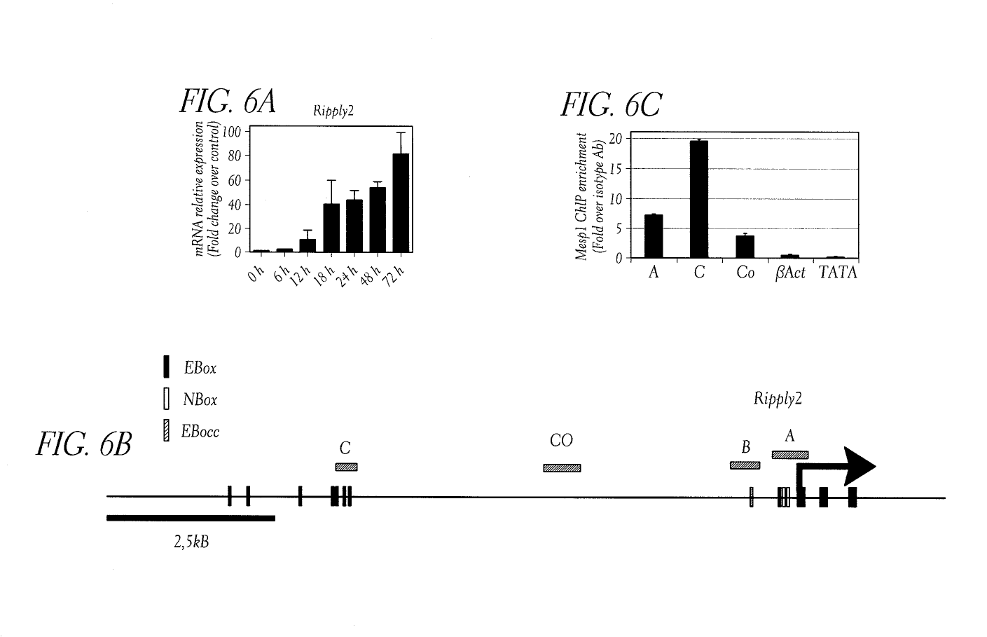

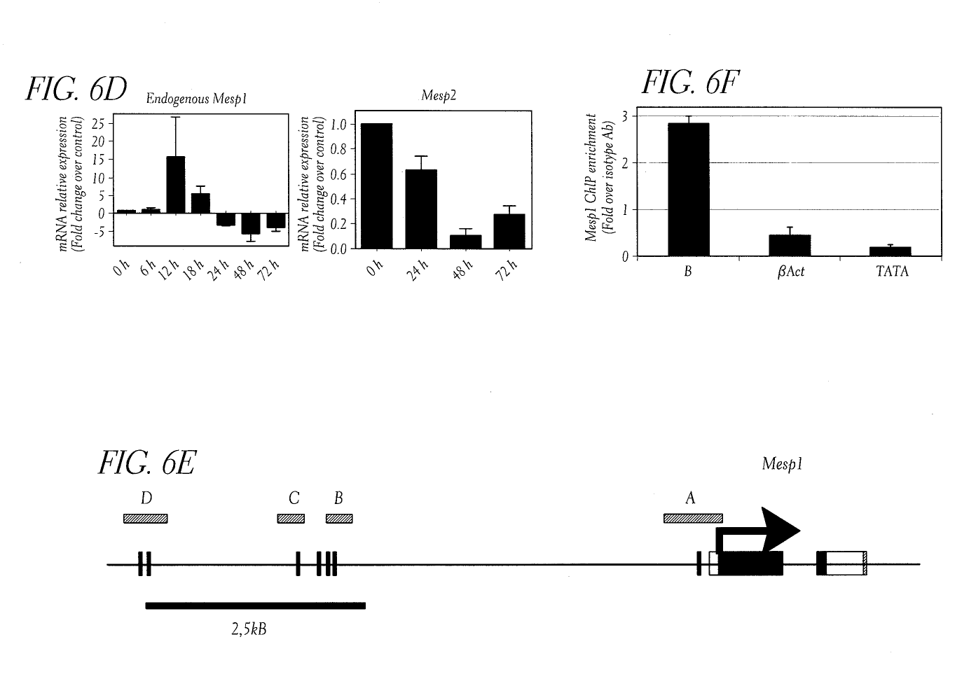

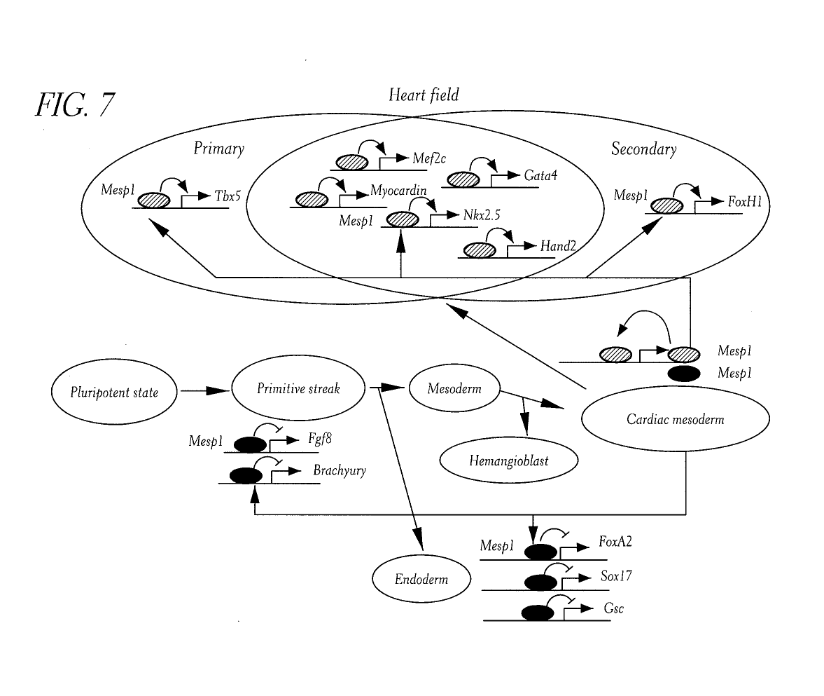

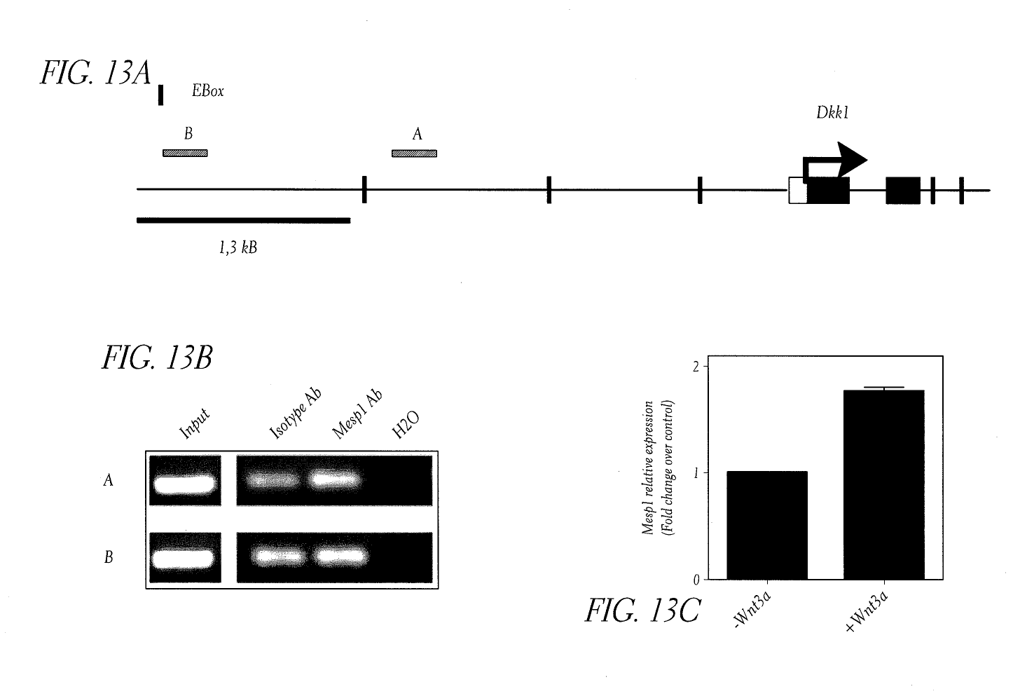

It has been recently suggested that Mesp1 promotes cardiac differentiation through the upregulation of Dkk1, a soluble Wnt inhibitor (David et al., 2008, Nat Cell Biol 10, 338-345). Our microarray and RT-PCR analysis revealed that Mesp1 promoted the expression of Lef1, a transcription factor that relays canonical Wnt signaling, and Wnt5a, a ligand of the non canonical Wnt pathway, and decreased the expression of Wnt3a, a ligand of the canonical Wnt pathway (Table 3). We did not detect any change in Dkk1 expression following Mesp1 induction, neither by micro-array nor by real time RT-PCR (FIG. 6F). We could not detect any enrichment of Mesp1 bound to Dkk1 promoter by ChIP, in the DNA region recently identified (FIGS. 13A and B). We found, as previously reported (Kwon et al., 2007, Proc Natl Acad Sci USA 104, 10894-10899; Lindsley et al., 2006, Development 133, 3787-3796; Naito et al., 2006, Proc Natl Acad Sci USA 103, 19812-19817; Ueno et al., 2007, Proc Natl Acad Sci USA 104, 9685-9690), that Dkk1 addition during ESC differentiation profoundly inhibits cardiac differentiation as measured by the number of beating EBs) and the number of troponinT positive cells. Addition of Dkk1, during Mesp1 induction, also decreased significantly but not completely the cardiac promoting effect of Mesp1 (FIGS. 6G-I). We confirmed the promoting effect of Wnt3a addition on Mesp1 expression (FIG. 13C) (Ueno et al., 2007, Proc Natl Acad Sci USA 104, 9685-9690), and demonstrate that addition of Dkk1 from D2 to D4 profoundly inhibited Mesp1 expression, suggesting that Wnt signaling can act upstream of Mesp1 expression and potentially explains why Wnt signaling is important during cardiovascular specification. Our results reinforce the prevaling notion that stimulation of canonical Wnt signaling is required for cardiac progenitor specification and/or expansion (Klaus et al., 2007, Proc Natl Acad Sci USA 104, 18531-18536; Kwon et al., 2007, Proc Natl Acad Sci USA 104, 10894-10899; Lindsley et al., 2006, Development 133, 3787-3796; Naito et al., 2006, Proc Natl Acad Sci USA 103, 19812-19817; Qyang et al., 2007, Cell Stem Cell 1, 165-179; Ueno et al., 2007, Proc Natl Acad Sci USA 104, 9685-9690) but clearly demonstrated that Dkk1 is not a direct Mesp1 target gene, and that Dkk1 is not responsible for the cardiac promotion mediated by Mesp1. Interestingly, while Dkk1 addition in early stage of ESC differentiation inhibits cardiac specification, Dkk1 addition during the latter stage promotes cardiac differentiation, suggesting that Wnt signaling present a biphasic effect during cardiac differentiation (Naito et al., 2006, Proc Natl Acad Sci USA 103, 19812-19817; Ueno et al., 2007, Proc Natl Acad Sci USA 104, 9685-9690; Yang et al., 2008, Nature).

### Example 8

### Mesp-1 Regulates its Own Expression Through a Complex Gene Regulatory Circuit

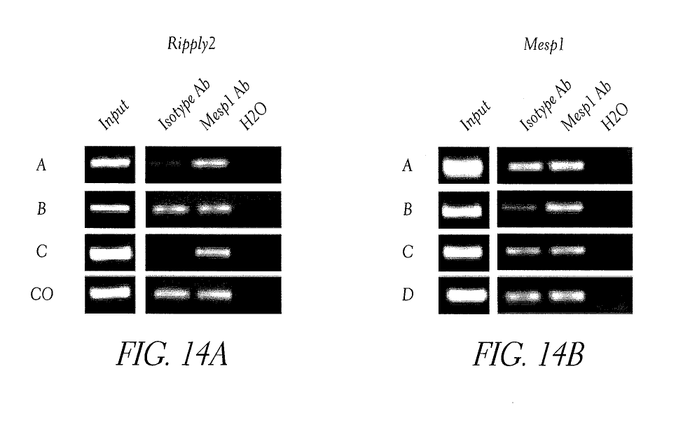

Mesp1 and Mesp2 are well known to repress each others expression and possibly also their own expression (Kitajima et al., 2000, Development 127, 3215-3226). Positive and negative auto-regulatory loops are common mechanisms during cell fate specification, ensuring a sharp boundary of gene expression as well as transient gene expression (Alon, 2007, Nat Rev Genet 8, 450-461). Our microarray analysis revealed that Ripply2, a well known direct negative regulator of Mesp2 expression (Kawamura et al., 2008, Mol Cell Biol 28, 3236-3244; Morimoto et al., 2007, Development 134, 1561-1569), was the most upregulated gene (about 50 fold) following Mesp1 induction (Table 3). Ripply2 expression is barely detectable at D3 of ESC differentiation, and its expression was very rapidly and strongly upregulated following Mesp1 induction (FIG. 6A). We investigated using ChIP experiments whether Mesp1 directly binds to Ripply2 promoter. Our ChIP experiments revealed that Mesp1 Ab enriches, by 20 fold, DNA fragments located 6.5 kB upstream of the ATG of Ripply2, and containing a cluster of four conserved Ebox sites (FIGS. 6B, 6C and 14A). To determine whether Mesp1 regulates its own expression, we measured the endogenous level of Mesp1 following Mesp1 induction, by designing RT-PCR primers specific for the 3′UTR region of endogenous Mesp1 mRNA that do not amplify the Mesp1 mRNA of the inducible construct. Interestingly, Mesp1 expression initially stimulated its own expression, but this effect was only transient, and after 24 hours following Dox addition, the endogenous expression of Mesp1 and Mesp2 were down-regulated in Mesp1 stimulated cells (FIG. 6D). The rapid and transient stimulation of Mesp1 expression following exogenous Mesp1 induction strongly suggested that Mesp1 first stimulated its own expression through a direct positive feedback loop. We used ChIP experiments to determine whether Mesp1 binds directly to its own regulatory region. Our ChIP experiments revealed that Mesp1 Ab enriched for DNA fragments located 4.6 kB upstream of the transcription initiation site of Mesp1 (FIGS. 6E, 6F and 14B), suggesting a direct auto-regulation of Mesp1. The subsequent downregulation of Mesp1 expression suggested an indirect mechanism, possibly mediated by the increase of Ripply2 expression.

To uncover the molecular mechanisms by which Mesp1 induced cardiovascular specification, we performed a genome wide analysis of Mesp1 regulated genes. We determined which genes were regulated upon Mesp1 induction, by at least 1.5 fold (see Table 3). A functional annotation clustering of Mesp1 regulated genes revealed that Mesp1 preferentially regulated genes implicated in morphogenesis and development (enrichment score=16.1 fold), tube morphogenesis and vessel development (7 fold), membrane proteins (6.3 fold), cell migration (5.3 fold), transcriptional regulation (4.8 fold), or negative regulation of physiological process (4.6 fold). The complete results are given in Table 3 below, further indicating relevant characteristics of said genes.

TABLE 3
Microarray Data

| | Upregualted genes | Downregulated genes |
|---|---|---|
| Transcription | Ripply2 (43.7), Cited1 (36.8), Trim9 (22.7), Foxl2os | T (8.5), Foxa2 (6.3), Sox17 |
| factors | (17.9), Hey2 (13.8), Otx1 (13.6), dHand (6.7), Ebf2 | (6.2), Ldb2 (4.6), Klhl4 (4.3), |
| | (6.3), Lhfp (5.5), Snail (4.9), Lef1 (4.3), Nfatc1 (4.0), | Gsc (3.3), Sp8 (3.1), Id2 (2.7), |
| | Pdlim4 (4.0), Myocd (3.7), Pdlim2 (3.5), Asxl3 (3.3), | Eras (2.7), Mixl1 (2.4), Zic5 |
| | Foxc1 (3.2), sVax1 (3.1), Twist1 (3.1), Fli1 (2.9), Fosl2 | (2.4), Irf6 (1.9), Foxd3 (2.1), |
| | (2.8), KlHl6 (2.8), Zeb1 (2.7), Ankrd6 (2.6), Insm1 | Bhlhb2 (2.1), Nr5a2 (1.9), |
| | (2.6), Gata4 (2.4), Hes6 (2.6), Spic (2.5), Hmga2 (2.5), | Hopx (1.9), Tox3 (1.8), |
| | Gbx2 (2.3), Pdlim5 (2.3), Dmrta1 (2.3), Ankrd1 (2.3), | Nkx6.3 (1.8), Prdm1 (1.8), |
| | sFOG (2.3), Hmgn3 (2.2), Dact1 (2.1), Zfp711 (2.1), | Tcfcp2l1 (1.8), Dmrt1 (1.8), |
| | Pbx1 (2.1), Zfp238 (2.1), Specc1 (2.1), Hdgfrp3 (2.1), | Esrrb (1.7), Mycl1 (1.7), |
| | Dachshund1 (2.1), Etv2 (2.0), Tshz1 (2.0), Hoxd13 | Mcf2l (1.7), Nr0b1 (1.7), |
| | (2.0), Tox (1.9), Lmo1 (1.9), Tbx20 (1.9), Creb3l2 | Pycard (1.7), Ltbp4 (1.6), |
| | (1.9), Tbx3 (1.9), Lbx1 (1.9), Gata6 (1.8), Phc2 (1.8), | Mybl2 (1.6), Klf2 (1.6), Pcaf |
| | L3mbtl3 (1.8), Neurod1 (1.7), Foxh1 (1.7), Dicer (1.7), | (1.5) |
| | Tceal1 (1.7), Fhl1 (1.7), Sap30l (1.6), Ppfibp1 (1.6), | |
| | Zeb2 (1.6), Cbx2 (1.6), Sox4 (1.6), Smad1 (1.6), Pitx2 | |
| | (1.6) | |
| Signalling | Notch: Ligands: Dll3 (12.3), Dll1 (3.2), Dlk1 (3.0). | |
| | Receptors: Notch1 (2.5) Notch4 (2.0) Notch3 (1.6) | |
| | Downstream Transcription factors: Hey2 (13.8), Hes6 | |
| | (2.6) | |
| | Wnt: Wnt5a (6.5), Lef1 (4.3) | Frzb (3.1) |
| | FGF: Fgf3 (3.5) | Fgf8 (5.5), Fgf5 (2.6), Fgf17 |
| | | (1.9) |
| | TGF-b: Tgfb1i1 (3.4), Fstl1 (2.9) | Fst (5.1), Cer1 (3.9), Nodal |
| | | (1.5) |
| | Ras: | Rab25 (2.2), Shb (2.8) |
| | Rhob (2.6), Rasl11b (2.3), Rasgrp3 (21.3), Rragd (1.7) | |
| Cardiac | Kctd12 (11.0), Myl1 (8.3), Fbn2 (4.3), Myl7 (3.6), | Mylpf (2.1), |
| conduction and | Clcn2, Myo1b (1.9), Mylip (1.9), Kctd6 (1.9), Kctd15 | |
| muscle fiber | (1.7), Kcnmb4 (1.6), Tpm1 (1.5) | |
| contraction | | |

What is claimed is:

1. A method of inducing, of enhancing the induction, or of differentiating stem cells into cardiovascular precursor or progenitor cells comprising the steps of:
a) transiently inducing the expression of the Mesp1 gene in said stem cells, and
b) culturing said induced stem cells in vitro thereby obtaining differentiated stem cells that are enriched in cardiovascular progenitor cells.
c) specifying and differentiating the cardiovascular progenitors generated by method of the invention into a particular subset of cardiovascular lineages such as cardiomyocytes, vascular cells or endothelial cells.

2. The method of claim 1, wherein the transient expression is performed in vitro by transforming said stem cells with a vector comprising the gene sequence of the Mesp-1 protein.

3. The method of claim 2, wherein said Mesp-1 gene sequence is placed in an inducible expression cassette.

4. The method of claim 3, wherein the inducible expression cassette is chosen from the group of the Tetracyclin or doxycyclin induced systems, Rheo switch systems, IPTG-LAC inducible systems, ecdysone inducible systems, or the cumate repressor/operator systems, inducible activation of modulator systems.

5. The method of claim 1, wherein the induction of the Mesp-1 expression is performed during cardiovascular competence which need to be precise for each types of stem cells used and that correspond for murine ESC to day 2 or day 3, or day 2 and day 3 of the culturing period of the stem cells.

6. The method of claims 5, wherein the induction is performed for one or two days only.

7. The method of claim 1, wherein the stem cells are selected from the group of: Embryonic Stem cells (ES), pluripotent stem cells, haematopoietic stem cells, totipotent stem cells, mesenchymal stem cells, induced pluripotent stem cells (iPS) or adult stem cells, adult heart, epicardial, vessel or muscular cells.

8. A method for performing cellular therapy for restoring the heart or vasculature function in a subject in need thereof, comprising the steps of:
a) providing cells according to the method of claim 1, and
b) injecting said cells into the heart or the vasculature of the subject in need thereof, wherein said cardiovascular function is preferably disturbed due to a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.

9. A method for restoring the heart or vasculature function in a subject in need thereof, in an endogenous manner, comprising the step of transiently inducing the expression of the Mesp-1 protein in the cells of the heart or the vasculature, wherein said cardiovascular function is preferably disturbed due to a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.

10. The method of claim 9, wherein said induction is performed by injecting the subject with an amount of an expression vector encoding for the Mesp-1 protein.

11. The method of claim 9, wherein said induction is performed by injecting the subject with an amount of an expression vector encoding for the Mesp-1 protein packed in a virus.

12. An assay for assessing the toxicity of an agent on heart or vascular cells, comprising the steps of:
a) differentiating stem cells into cardiovascular progenitor cells according to the method of claim 1,
b) subjecting said cells in vitro to said agent, and
c) analysing the toxic effect of said agent on the cells obtained in step a).

13. An assay for assessing the pharmacology of a candidate drug comprising the steps of:
a) differentiating stem cells into cardiovascular progenitor cells according to the method of claim 1,
b) subjecting said cells in vitro to said candidate drug, and
c) analysing the behaviour of said cells in the presence and absence of said candidate drug.

14. A method for identifying target genes for therapy of cardiovascular disorders comprising the steps of:
a) differentiating stem cells into cardiovascular progenitor cells according to the method of claim 1,
b) analysing the expression level of the genes in said cells prior to and after said induction of Mesp-1 expression in said stem cells,
wherein genes that are up-regulated after the gene-induction are putative targets for stimulation of differentiation of cardiovascular differentiation and those genes that are down-regulated after the gene-induction are putative targets for inhibiting cardiovascular differentiation of stem cells.

15. Cardiovascular progenitor cells obtained by the method of claim 1.

16. Cardiovascular cells obtained by the method of claim 1.

17. A method of diagnosis and/or treatment of congenital heart diseases comprising the detection of the occurrence of mutations in the Mesp1 genomic.


SEQUENCE LISTING (236 sequences)

SEQ ID NO: 1 - DNA, 25 bp, artificial sequence
  Beta-Actin
accaactgggacgatatggagaaga

SEQ ID NO: 2 - DNA, 25 bp, artificial sequence
  Beta-Actin
tacgaccagaggcatacagggacaa

SEQ ID NO: 3 - DNA, 26 bp, artificial sequence
  TATA-BP
tgtaccgcagcttcaaaatattgtat

SEQ ID NO: 4 - DNA, 21 bp, artificial sequence
  TATA-BP
aaatcaacgcagttgtccgtg

SEQ ID NO: 5 - DNA, 20 bp, artificial sequence
  Brachyury
ctccaacctatgcggacaat

SEQ ID NO: 6 - DNA, 20 bp, artificial sequence
  Brachyury
ccccttcatacatcggagaa

SEQ ID NO: 7 - DNA, 20 bp, artificial sequence
  Mesp1 (ORF-3 prime UTR)
tgtacgcagaaacagcatcc

SEQ ID NO: 8 - DNA, 20 bp, artificial sequence
  Mesp1 (ORF-3 prime UTR)
ttgtcccctccactcttcag

SEQ ID NO: 9 - DNA, 20 bp, artificial sequence
  Nkx2.5
ctccgatccatcccacttta

SEQ ID NO: 10 - DNA, 20 bp, artificial sequence
  Nkx2.5
agtgtggaatccgtcgaaag

SEQ ID NO: 11 - DNA, 19 bp, artificial sequence
  Gata4
ccacgggccctccatccat

SEQ ID NO: 12 - DNA, 19 bp, artificial sequence
  Gata4
ggcccccacgtcccaagtc

SEQ ID NO: 13 - DNA, 20 bp, artificial sequence
  Hand2
cccgccgacaccaaactctc

SEQ ID NO: 14 - DNA, 19 bp, artificial sequence
  Hand2
cccccggctcactgctctc

SEQ ID NO: 15 - DNA, 20 bp, artificial sequence
  Mef2c
aggcaccagcgcagggaatg

SEQ ID NO: 16 - DNA, 21 bp, artificial sequence
  Mef2c
ccaccggggtagccaatgact

SEQ ID NO: 17 - DNA, 24 bp, artificial sequence
  TroponinT2 (cTnT)
gcggaagagtgggaagagacagac

SEQ ID NO: 18 - DNA, 20 bp, artificial sequence
  TroponinT2 (cTnT)
gcacggggcaaggacacaag

SEQ ID NO: 19 - DNA, 21 bp, artificial sequence
  Hand1
ggtcggcaggtccttcgtgtc

SEQ ID NO: 20 - DNA, 19 bp, artificial sequence
  Hand1
gtgcggcgggtgtgagtgg

SEQ ID NO: 21 - DNA, 24 bp, artificial sequence
  Tbx1
cagccccgattccatgttgtctat

SEQ ID NO: 22 - DNA, 19 bp, artificial sequence
  Tbx1
ggttccggggccagtcctc

SEQ ID NO: 23 - DNA, 21 bp, artificial sequence
  Tbx5
ctaccccgcgcccactctcat

SEQ ID NO: 24 - DNA, 20 bp, artificial sequence
  Tbx5
tgcggtcggggtccaacact

SEQ ID NO: 25 - DNA, 20 bp, artificial sequence
  Tbx20
atcgccgcgcttatgtccag

SEQ ID NO: 26 - DNA, 17 bp, artificial sequence
  Tbx20
ccccgccgccaaactcc

SEQ ID NO: 27 - DNA, 24 bp, artificial sequence
  Islet1
agcagcaacccaacgacaaaacta

SEQ ID NO: 28 - DNA, 23 bp, artificial sequence
  Islet1
gtatctgggagctgcgaggacat

SEQ ID NO: 29 - DNA, 22 bp, artificial sequence
  AlphaMHC
gctgggctccctggacattgac

SEQ ID NO: 30 - DNA, 22 bp, artificial sequence
  AlphaMHC
cctgggcctggattctggtgat

SEQ ID NO: 31 - DNA, 24 bp, artificial sequence
  Mlc2a
aagggaagggtcccatcaacttca

SEQ ID NO: 32 - DNA, 24 bp, artificial sequence
  Mlc2a
aacagttgctctacctcagcagga

SEQ ID NO: 33 - DNA, 24 bp, artificial sequence
  Mlc2v
acttcaccgtgttcctcacgatgt

SEQ ID NO: 34 - DNA, 24 bp, artificial sequence
  Mlc2v
tccgtgggtaatgatgtggaccaa

SEQ ID NO: 35 - DNA, 23 bp, artificial sequence
  ANF
accctgggcttcttcctcgtctt

SEQ ID NO: 36 - DNA, 18 bp, artificial sequence
  ANF
gcggcccctgcttcctca

SEQ ID NO: 37 - DNA, 23 bp, artificial sequence
  Kcne1
ctgggcttcttcggcttcttcac

SEQ ID NO: 38 - DNA, 22 bp, artificial sequence
  Kcne1
ctacggccgcctggttttcaat

SEQ ID NO: 39 - DNA, 22 bp, artificial sequence
  CD31
aatggcaactggagcgagcact

SEQ ID NO: 40 - DNA, 23 bp, artificial sequence
  CD31
ggagaaggcgaggagggttaggt

SEQ ID NO: 41 - DNA, 20 bp, artificial sequence
  Gata1
gtggcggagggacaggacag

SEQ ID NO: 42 - DNA, 22 bp, artificial sequence
  Gata1
gattcgacccccgcttcttttt

SEQ ID NO: 43 - DNA, 21 bp, artificial sequence
  Tie2
ggagccgcggactgactacga

SEQ ID NO: 44 - DNA, 21 bp, artificial sequence
  Tie2
cctgcgccttggtgttgactc

SEQ ID NO: 45 - DNA, 18 bp, artificial sequence
  Runx2
aggccgccgcacgacaac

SEQ ID NO: 46 - DNA, 20 bp, artificial sequence
  Runx2
cgctccggcccacaaatctc

SEQ ID NO: 47 - DNA, 21 bp, artificial sequence
  Col1a1
gtccccctggctctgctggtt

SEQ ID NO: 48 - DNA, 20 bp, artificial sequence
  Col1a1
tcggggctgcggatgttctc

SEQ ID NO: 49 - DNA, 24 bp, artificial sequence
  Albumin1
cttcgtctccggctctgctttttc

SEQ ID NO: 50 - DNA, 24 bp, artificial sequence
  Albumin1
cgtttctttcgggctcttgttttg

SEQ ID NO: 51 - DNA, 22 bp, artificial sequence
  AFP
gagaaatggtccggctgtggtg

SEQ ID NO: 52 - DNA, 21 bp, artificial sequence
  AFP
gggggaggggcataggtttca

SEQ ID NO: 53 - DNA, 22 bp, artificial sequence
  TCF1
ctccagccaccaccatccacat

SEQ ID NO: 54 - DNA, 18 bp, artificial sequence
  TCF1
gctgcggccctcctcacc

SEQ ID NO: 55 - DNA, 24 bp, artificial sequence
  Pdx1
acctaggcgtcgcacaagaagaaa

SEQ ID NO: 56 - DNA, 21 bp, artificial sequence
  Pdx1
gctcgcctgctggtccgtatt

SEQ ID NO: 57 - DNA, 19 bp, artificial sequence
  Sox17
gcggcgcaagcaggtgaag

SEQ ID NO: 58 - DNA, 19 bp, artificial sequence
  Sox17
ggggcccatgtgcggagac

SEQ ID NO: 59 - DNA, 18 bp, artificial sequence
  Sox18
gcgcagccccgaatcagg

SEQ ID NO: 60 - DNA, 19 bp, artificial sequence
  Sox18
cgaggcccgggagcaagag

SEQ ID NO: 61 - DNA, 19 bp, artificial sequence
  Gata6
gccgcaccgctgactcctg

SEQ ID NO: 62 - DNA, 22 bp, artificial sequence
  Gata6
acgcgcttctgtggcttgatga

SEQ ID NO: 63 - DNA, 17 bp, artificial sequence
  K8
tggcggggctggtgtcg

SEQ ID NO: 64 - DNA, 19 bp, artificial sequence
  K8
ctgccggcggaggttgttg

SEQ ID NO: 65 - DNA, 18 bp, artificial sequence
  K14
gccgcccctggtgtggac

SEQ ID NO: 66 - DNA, 21 bp, artificial sequence
  K14
gtgcgccggagctcagaaatc

SEQ ID NO: 67 - DNA, 24 bp, artificial sequence
  K18
tggactccgcaaggtggtagatga

SEQ ID NO: 68 - DNA, 24 bp, artificial sequence
  K18
atttcggcagacttggtggtgaca

SEQ ID NO: 69 - DNA, 21 bp, artificial sequence
  Beta3-tubulin
ccagtgcggcaaccagatagg

SEQ ID NO: 70 - DNA, 21 bp, artificial sequence
  Beta3-tubulin
aaaggcgccagaccgaacact

SEQ ID NO: 71 - DNA, 20 bp, artificial sequence
  Nestin
cggagagggagcagcaccaa

SEQ ID NO: 72 - DNA, 20 bp, artificial sequence
  Nestin
ggcctcccccacagcatcct

SEQ ID NO: 73 - DNA, 20 bp, artificial sequence
  Sox1
gcgccctcggatctctggtc

SEQ ID NO: 74 - DNA, 18 bp, artificial sequence
  Sox1
gcccgcgccctggtagtg

SEQ ID NO: 75 - DNA, 24 bp, artificial sequence
  Islet1
agcagcaacccaacgacaaaacta

SEQ ID NO: 76 - DNA, 23 bp, artificial sequence
  Islet1
gtatctgggagctgcgaggacat

SEQ ID NO: 77 - DNA, 24 bp, artificial sequence
  Myocardin
agtgggcccagcattttcaacatc

SEQ ID NO: 78 - DNA, 22 bp, artificial sequence
  Myocardin
ccctccccattttccccacttc

SEQ ID NO: 79 - DNA, 20 bp, artificial sequence
  FoxH1
ggggcctcgcgacaactctc

SEQ ID NO: 80 - DNA, 24 bp, artificial sequence
  Foxc1
cactgcctggacctgacggataat

SEQ ID NO: 81 - DNA, 20 bp, artificial sequence
  Foxc1
ccggcccctatgagcgtgta

SEQ ID NO: 82 - DNA, 22 bp, artificial sequence
  Foxc1
cttcttgtccggggcattctgg

SEQ ID NO: 83 - DNA, 24 bp, artificial sequence
  Foxc2
ccagaacgcgccagagaagaagat

SEQ ID NO: 84 - DNA, 19 bp, artificial sequence
  Foxc2
ccgccgccgccgcaggaag

SEQ ID NO: 85 - DNA, 20 bp, artificial sequence
  Oct4
ccaatcagcttgggctagag

SEQ ID NO: 86 - DNA, 20 bp, artificial sequence
  Oct4
tgtctacctcccttgccttg

SEQ ID NO: 87 - DNA, 21 bp, artificial sequence
  Nanog
gcctctcctcgcccttcctct

SEQ ID NO: 88 - DNA, 22 bp, artificial sequence
  Nanog
ccaccgcttgcacttcatcctt

SEQ ID NO: 89 - DNA, 20 bp, artificial sequence
  Sox2
accggcggcaaccagaagaa

SEQ ID NO: 90 - DNA, 21 bp, artificial sequence
  Sox2
caagcctccgggaagcgtgta

SEQ ID NO: 91 - DNA, 22 bp, artificial sequence
  Eras
tgctgggcgtctttgctcttga

SEQ ID NO: 92 - DNA, 22 bp, artificial sequence
  Eras
cctcctgggccctctgaatctc

SEQ ID NO: 93 - DNA, 21 bp, artificial sequence
  Id2
gccgctgaccaccctgaacac

SEQ ID NO: 94 - DNA, 23 bp, artificial sequence
  Id2
agaacgacacctgggcaagacga

SEQ ID NO: 95 - DNA, 22 bp, artificial sequence
  Foxa2
ctgaagcccgagcaccattacg

SEQ ID NO: 96 - DNA, 20 bp, artificial sequence
  Foxa2
atccaggcccgctttgttcg

SEQ ID NO: 97 - DNA, 18 bp, artificial sequence
  FGF8
tcagccgccgcctcatcc

SEQ ID NO: 98 - DNA, 24 bp, artificial sequence
  FGF8
cagcgccgtgtagttgttctccag

SEQ ID NO: 99 - DNA, 20 bp, artificial sequence
  GSC
acgagggccccggttctgta

SEQ ID NO: 100 - DNA, 24 bp, artificial sequence
  GSC
cacttctcggcgttttctgactcc

SEQ ID NO: 101 - DNA, 23 bp, artificial sequence
  Cer1
cagcagatgggaggaaagtggag

SEQ ID NO: 102 - DNA, 19 bp, artificial sequence
  Cer1
gggcgagcagtgggagcag

SEQ ID NO: 103 - DNA, 18 bp, artificial sequence
  Nodal
gggcggatggggcagaag

SEQ ID NO: 104 - DNA, 20 bp, artificial sequence
  Nodal
ccggtggggttggtatcgtt

SEQ ID NO: 105 - DNA, 20 bp, artificial sequence
  Dll1
cgccttcagcaaccccatcc

SEQ ID NO: 106 - DNA, 22 bp, artificial sequence
  Dll1
ctgtgcggccgctactgtgaag

SEQ ID NO: 107 - DNA, 21 bp, artificial sequence
  Dll3
gcacgccattcccagacgagt

SEQ ID NO: 108 - DNA, 21 bp, artificial sequence
  Dll3
ccggggacaggcacattcaaa

SEQ ID NO: 109 - DNA, 21 bp, artificial sequence
  Dll4
gcggcatgcctgggaagtatc

SEQ ID NO: 110 - DNA, 19 bp, artificial sequence
  Dll4
gggccggagctgggtgtct

SEQ ID NO: 111 - DNA, 21 bp, artificial sequence
  Notch1
agggggaggtggatgctgact

SEQ ID NO: 112 - DNA, 22 bp, artificial sequence
  Notch1
gctggcgccctggtagatgaag

SEQ ID NO: 113 - DNA, 20 bp, artificial sequence
  Notch4
ggctgccccctggtttcatt

SEQ ID NO: 114 - DNA, 20 bp, artificial sequence
  Notch4
tcttcagggcccgagcacat

SEQ ID NO: 115 - DNA, 23 bp, artificial sequence
  Hes6
gccagggggtgcactaaagaaag

SEQ ID NO: 116 - DNA, 17 bp, artificial sequence
  Hes6
gcccgcctcccctggtc

SEQ ID NO: 117 - DNA, 22 bp, artificial sequence
  Hey2
ccgaaagcgacctggacgagac

SEQ ID NO: 118 - DNA, 22 bp, artificial sequence
  Hey2
accccctgtagcctggagcatc

SEQ ID NO: 119 - DNA, 21 bp, artificial sequence
  Wnt3a
cacccgggagtcagcctttgt

SEQ ID NO: 120 - DNA, 20 bp, artificial sequence
  Wnt3a
gcgcccagcctcattgttgt

SEQ ID NO: 121 - DNA, 21 bp, artificial sequence
  Wnt5a
cgggagggcgagctgtctacc

SEQ ID NO: 122 - DNA, 24 bp, artificial sequence
  Wnt5a
cctacggcctgcttcattgttgtg

SEQ ID NO: 123 - DNA, 20 bp, artificial sequence
  Dkk1
gctgccccgggaactactgc

SEQ ID NO: 124 - DNA, 24 bp, artificial sequence
  Dkk1
gagccttcttgtcctttggtgtga

SEQ ID NO: 125 - DNA, 19 bp, artificial sequence
  Ripply2
cgggtccgagggcttctgg

SEQ ID NO: 126 - DNA, 21 bp, artificial sequence
  Ripply2
gccccgtccgcttctctttct

SEQ ID NO: 127 - DNA, 20 bp, artificial sequence
  Mesp2
cgcctggccatccgctacat

SEQ ID NO: 128 - DNA, 23 bp, artificial sequence
  Mesp2
cacccccaggacaccccactact

SEQ ID NO: 129 - DNA, 21 bp, artificial sequence
  cHand2AF1
agagaaggcctcggcggtaac

SEQ ID NO: 130 - DNA, 23 bp, artificial sequence
  cHand2AR1
tctaaacagaaagggggcgagag

SEQ ID NO: 131 - DNA, 18 bp, artificial sequence
  cHand2BF1
cccgggattggcgtgagg

SEQ ID NO: 132 - DNA, 21 bp, artificial sequence
  cHand2BR1
gaagcggcgaatggactctcg

SEQ ID NO: 133 - DNA, 30 bp, artificial sequence
  cHand2CF1
tcataaaaactagaaaataagctccgaaca

SEQ ID NO: 134 - DNA, 26 bp, artificial sequence
  cHand2CF1
gttaggatgacaacttgcagagaacg

SEQ ID NO: 135 - DNA, 30 bp, artificial sequence
  cHand2DF1
aggacataatcatcttacccagtctacctg

SEQ ID NO: 136 - DNA, 28 bp, artificial sequence
  cHand2DR1
ttgatggcagaacaaagtgacctacata

SEQ ID NO: 137 - DNA, 26 bp, artificial sequence
  cHand2EF1
ctccagccacctacagaacgctatcc

SEQ ID NO: 138 - DNA, 30 bp, artificial sequence
  cHand2ER1
accaccaacaacaacaaaaagtaggggtat

SEQ ID NO: 139 - DNA, 30 bp, artificial sequence
  cHand2FF1
tgaggagtcttcccaatcagtttactacct

SEQ ID NO: 140 - DNA, 27 bp, artificial sequence
  cHand2FR1
aaaggcaagggcaattttgtatctcgt

SEQ ID NO: 141 - DNA, 32 bp, artificial sequence
  cHand2GF1
aatgcacactctgaaaaagactcatgacattg

SEQ ID NO: 142 - DNA, 31 bp, artificial sequence
  cHand2GR1
aaaaacattggtaaagttagcactgggatac

SEQ ID NO: 143 - DNA, 32 bp, artificial sequence
  cHand2HF1
cagaaagttcagagaatggaaggcttgatatg

SEQ ID NO: 144 - DNA, 32 bp, artificial sequence
  cHand2HR1
ggcttaccttcccaagctagagaagaacctac

SEQ ID NO: 145 - DNA, 30 bp, artificial sequence
  cHand2IF1
agaaaggaagctgaagaaacaacactggtg

SEQ ID NO: 146 - DNA, 26 bp, artificial sequence
  cHand2IR1
aaagggctctcagcgtggagttagac

SEQ ID NO: 147 - DNA, 23 bp, artificial sequence
  cHand2JF1
cccaacaccgaggggagactacc

SEQ ID NO: 148 - DNA, 31 bp, artificial sequence
  cHand2JR1
gctgttaaactgctagcatgatttgtcgtat

SEQ ID NO: 149 - DNA, 29 bp, artificial sequence
  cHand2KF1
aacaatgccttacggttattttcatagtc

SEQ ID NO: 150 - DNA, 32 bp, artificial sequence
  cHand2KR1
taatactccaaggcataggaaatcgtagttag

SEQ ID NO: 151 - DNA, 32 bp, artificial sequence
  cHand2LR1
ctgtcagtcagcagaaataaagaatcctattg

SEQ ID NO: 152 - DNA, 32 bp, artificial sequence
  cHand2LR1
agaacacttctttggaattcctttttggatac

SEQ ID NO: 153 - DNA, 32 bp, artificial sequence
  cHand2MF1
tatcagccagttatttccaagtatcagagtta

SEQ ID NO: 154 - DNA, 32 bp, artificial sequence
  cHand2MR1
cagacatttttatttcttttcctccgctcata

SEQ ID NO: 155 - DNA, 23 bp, artificial sequence
  cMyocAF
aaaaatatttgtggcgctggttt

SEQ ID NO: 156 - DNA, 27 bp, artificial sequence
  cMyocAR
ttatagagtgggcgcgttatcagttac

SEQ ID NO: 157 - DNA, 17 bp, artificial sequence
  cMyocBF
acgcggccccaggagtc

SEQ ID NO: 158 - DNA, 19 bp, artificial sequence
  cMyocBR
tcggatacagaggggaagc

SEQ ID NO: 159 - DNA, 23 bp, artificial sequence
  cMyocCF
acagggtcccacgtgcatcatta

SEQ ID NO: 160 - DNA, 24 bp, artificial sequence
  cMyocCR
ggcctccacctgtcattgtcattc

SEQ ID NO: 161 - DNA, 23 bp, artificial sequence
  cMyocCOF
agggccgggcttttgcatctaac

SEQ ID NO: 162 - DNA, 22 bp, artificial sequence
  cMyocCOR
tgtgcctccatgtccagtgata

SEQ ID NO: 163 - DNA, 22 bp, artificial sequence
  cN24AF
atggtggcgacgcaggtttcac

SEQ ID NO: 164 - DNA, 21 bp, artificial sequence
  cN25AR
ggcccaatggcaggctgaatc

SEQ ID NO: 165 - DNA, 24 bp, artificial sequence
  cN25BF
acgggcagttctgcgtcacctaat

SEQ ID NO: 166 - DNA, 24 bp, artificial sequence
  cN25BR
tgggattttcaggctaacgaggag

SEQ ID NO: 167 - DNA, 24 bp, artificial sequence
  cN25C
aacggtaatatttcaggcgtcagc

SEQ ID NO: 168 - DNA, 17 bp, artificial sequence
  cN25C
gctggccctgcggatcg

SEQ ID NO: 169 - DNA, 24 bp, artificial sequence
  cN25D
agcggcccctttgttgatacagta

SEQ ID NO: 170 - DNA, 24 bp, artificial sequence
  cN25D
ctgcaatcagccgcgaaaagtata

SEQ ID NO: 171 - DNA, 23 bp, artificial sequence
  cN25E
acacggggaaagcccagactacg

SEQ ID NO: 172 - DNA, 22 bp, artificial sequence
  cN25E
gtgcactccggaattgtgaacg

SEQ ID NO: 173 - DNA, 23 bp, artificial sequence
  cG4AF
gaagcgaagcggcagtcctgaag

SEQ ID NO: 174 - DNA, 24 bp, artificial sequence
  cG4AR
gctgtactgggcgtccgttgaacc

SEQ ID NO: 175 - DNA, 27 bp, artificial sequence
  cG4BF
tcgtactgtgcatcatgtgggtgtcaa

SEQ ID NO: 176 - DNA, 27 bp, artificial sequence
  cG4BR
gagaggaaggattggaattaacaggtg

SEQ ID NO: 177 - DNA, 25 bp, artificial sequence
  cG4CF
caagcctgaaaaactgcaagcacta

SEQ ID NO: 178 - DNA, 27 bp, artificial sequence
  cG4CR
atgaacgttgtagggtgatttgaaaga

SEQ ID NO: 179 - DNA, 22 bp, artificial sequence
  cG4DF
caggggagcttgagccgactac

SEQ ID NO: 180 - DNA, 27 bp, artificial sequence
  cG4DR
tgcactgctgaatactacccacataca

SEQ ID NO: 181 - DNA, 22 bp, artificial sequence
  cG4EF
ttcccaaagctcccccaacagg

SEQ ID NO: 182 - DNA, 27 bp, artificial sequence
  cG4ER
ggaggaaagagaaggagaataaacacg

SEQ ID NO: 183 - DNA, 23 bp, artificial sequence
  cG4FF
tccaacagctgccaggcgacatt

SEQ ID NO: 184 - DNA, 26 bp, artificial sequence
  cG4FR
gaaaggaaaagcggtctgggatggtc

SEQ ID NO: 185 - DNA, 21 bp, artificial sequence
  cG4GF
cgaagggagggtgacgacgac

SEQ ID NO: 186 - DNA, 24 bp, artificial sequence
  cG4GR
ctgtgcggcttgtgagttgattcc

SEQ ID NO: 187 - DNA, 24 bp, artificial sequence
  cFoxA2AF
acctgaagcccgagcaccattacg

SEQ ID NO: 188 - DNA, 22 bp, artificial sequence
  cFoxA2AR
gcatccaggcccgctttgttcg

SEQ ID NO: 189 - DNA, 22 bp, artificial sequence
  cFoxA2BF
cgcgctgccaaacataactctg

SEQ ID NO: 190 - DNA, 24 bp, artificial sequence
  cFoxA2BR
gccgccttttccgccctccttcta

SEQ ID NO: 191 - DNA, 18 bp, artificial sequence
  cFoxA2CF
cggggcgctcgtagactt

SEQ ID NO: 192 - DNA, 21 bp, artificial sequence
  cFoxA2CR
tcggccccaggtgaggtttag

SEQ ID NO: 193 - DNA, 27 bp, artificial sequence
  cFoxA2DF
ttgtttggaagtctgggttttagttat

SEQ ID NO: 194 - DNA, 27 bp, artificial sequence
  cFoxA2DR
tcctcctcacggttctctgttgttatt

SEQ ID NO: 195 - DNA, 24 bp, artificial sequence
  cGSCAF1
ctcccggacccaagcctcacaact

SEQ ID NO: 196 - DNA, 23 bp, artificial sequence
  cGSCAR1
gggcatgcggggcgacaaacaat

SEQ ID NO: 197 - DNA, 24 bp, artificial sequence
  cGSCBF1
atcagcttctaacgttttcattca

SEQ ID NO: 198 - DNA, 22 bp, artificial sequence
  cGSCBR1
ctcttttagcagtttttcacaa

SEQ ID NO: 199 - DNA, 21 bp, artificial sequence
  cGSCCF1
tgagaacgggccacttactat

SEQ ID NO: 200 - DNA, 24 bp, artificial sequence
  cGSCCR1
tttggtgcgccttggactgatttc

SEQ ID NO: 201 - DNA, 24 bp, artificial sequence
  cSox17AF2
tccaggatttatttaagattgaga

SEQ ID NO: 202 - DNA, 19 bp, artificial sequence
  cSox17AR2
aactaccccgacatttgac

SEQ ID NO: 203 - DNA, 22 bp, artificial sequence
  cSox17BF2
ggggctggctctggtcgtcact

SEQ ID NO: 204 - DNA, 23 bp, artificial sequence
  cSox17BR2
aggagagcagcgggagggtagca

SEQ ID NO: 205 - DNA, 21 bp, artificial sequence
  cSox17cF2
cagacccgccagcagtgtgag

SEQ ID NO: 206 - DNA, 24 bp, artificial sequence
  cSox17cR2
ttggggatgtggcttaggcagtag

SEQ ID NO: 207 - DNA, 19 bp, artificial sequence
  cSox17DF2
gggggctcattccgcacac

SEQ ID NO: 208 - DNA, 20 bp, artificial sequence
  cSox17DR2
gatggggtatgggttctaag

SEQ ID NO: 209 - DNA, 24 bp, artificial sequence
  cSox17EF2
tgagccaggactaaatgagaatac

SEQ ID NO: 210 - DNA, 23 bp, artificial sequence
  cSox17ER2
tggagggcaggatgtggggttta

SEQ ID NO: 211 - DNA, 18 bp, artificial sequence
  cBrachAF
cgcgctggagcccattgt

SEQ ID NO: 212 - DNA, 24 bp, artificial sequence
  cBrachAR
gacaccctttgaagtaccgagcag

SEQ ID NO: 213 - DNA, 17 bp, artificial sequence
  cBrachBF
ggagggcgggggtgtcg

SEQ ID NO: 214 - DNA, 20 bp, artificial sequence
  cBrachBR
aggctgggggccaacaatgg

SEQ ID NO: 215 - DNA, 30 bp, artificial sequence
  cBrachCF
ttgactttcatgtcctcctccaccgagatt

SEQ ID NO: 216 - DNA, 30 bp, artificial sequence
  cBrachCR
ccctcctctgccctttcccactgaatactg

SEQ ID NO: 217 - DNA, 20 bp, artificial sequence
  cDkk1AF
gaatatggggagagaagtgg

SEQ ID NO: 218 - DNA, 21 bp, artificial sequence
  cDkk1AR
cagcatactactagcaatgtc

SEQ ID NO: 219 - DNA, 20 bp, artificial sequence
  cDkk1BF
gcttgtctatcacgatgagc

SEQ ID NO: 220 - DNA, 20 bp, artificial sequence
  cDkk1BR
gcaaagatttcccgttcctg

SEQ ID NO: 221 - DNA, 24 bp, artificial sequence
  cRply2AF
cgcatgctgtttttctcccagacc

SEQ ID NO: 222 - DNA, 22 bp, artificial sequence
  cRply2AR
ctcggcgctctcggtggtatcc

SEQ ID NO: 223 - DNA, 24 bp, artificial sequence
  cRply2BF
ttcccaaccacaaaagtatcgtct

SEQ ID NO: 224 - DNA, 24 bp, artificial sequence
  cRply2BR
tgttacttgaaggggatggacaat

SEQ ID NO: 225 - DNA, 24 bp, artificial sequence
  cRply2CF
cagctctgctcagttctgcgtcag

SEQ ID NO: 226 - DNA, 24 bp, artificial sequence
  cRply2CR
tttgaaactcacttgcccaaccaa

SEQ ID NO: 227 - DNA, 19 bp, artificial sequence
  cRply2COF
ggggaccatcagcatcacg

SEQ ID NO: 228 - DNA, 24 bp, artificial sequence
  cRply2COR
attcagcgactaaagggttctacg

SEQ ID NO: 229 - DNA, 20 bp, artificial sequence
  cM1AF
gggcctgaaccctttgaacc

SEQ ID NO: 230 - DNA, 24 bp, artificial sequence
  cM1AR
cctggccataggtgcctgacttac

SEQ ID NO: 231 - DNA, 23 bp, artificial sequence
  cM1BF
caggagagggaggctgtgaacga

SEQ ID NO: 232 - DNA, 24 bp, artificial sequence
  cM1BR
cacaggggcaacagtggtaacaga

SEQ ID NO: 233 - DNA, 24 bp, artificial sequence
  cM1CF
tttggggcctgtgttttgacaagt

SEQ ID NO: 234 - DNA, 24 bp, artificial sequence
  cM1CR
ggctgcagagtgggtgggagtatg

SEQ ID NO: 235 - DNA, 23 bp, artificial sequence
  cM1DF
actggccctcctcacacctctcg

SEQ ID NO: 236 - DNA, 24 bp, artificial sequence
  cM1DR
tggcccaggaccagataatcagat